(19) United States
(12) Patent Application Publication
(10) Pub. No.: US 2024/0197229 A1
(43) Pub. Date: Jun. 20, 2024

(54) Packet ECG for Heart Attack Diagnostics

(71) Applicant: VLADIMIR KRANZ, Prague 4 (CZ)

(72) Inventor: VLADIMIR KRANZ, Prague 4 (CZ)

(21) Appl. No.: 17/620,143

(22) PCT Filed: Jun. 29, 2020
(86) PCT No.: PCT/CZ2020/000034

§ 371 Date: Jan. 25, 2024

(30) Foreign Application Priority Data
Jun. 27, 2019 (CZ) PV 2019-428

(51) Int. Cl. A61B 5/282; A61B 5/256; A61B 5/332

(52) U.S. Cl. CPC A61B 5/282; A61B 5/256; A61B 5/332; A61B 2562/222

(57) ABSTRACT

The invention relates to a pocket monitor of cardiac signals with the possibility of preliminary detection of infarct symptoms and of subsequent more detailed verification. Preferably, the monitor is formed of a two-channel monitor located on the sensing unit, preferably formed by a “two-channel monitor base”. Underneath the base, three ECG electrodes are provided, preferably formed by electrodes for sensing of potential of a Kranz's terminal, an electrode for sensing of the V4 lead, and an electrode for sensing of the V5 lead. Their assembly is placed on the chest, what allows sensing of two chest leads at once, namely the V4 and the 5V leads.

### FIELD OF THE ART

This device relates to monitoring of heart activity

### PRIOR ART

Pocket devices for short-term operative monitoring of cardiac activity by a monitored person by placing fingers on a monitoring device or by placing a monitoring device on the chest according to the prior art are intended for sensing and displaying of a single-lead ECG only and they do not allow sensing of any of the chest leads V1 to V6 for finding of the possible ST elevation to reveal an acute myocardial infarction (AIM), and they do not provide any automatic evaluation of AIM to activate the alarm in case of its warning of the monitored person. This means that common population does not have available any pocket device to be carried by persons with them, which device could, especially in the event of symptoms, confirm or exclude an AIM, and which would transmit the result to the rescue service to speed up the life-saving action.

At the same time, ischemic heart disease, which causes AIM, is the most common cause of death worldwide, and is responsible for almost 1.8 million deaths a year, or 20% of all deaths in Europe actually. In the European countries, its incidence ranged from 43 to 144 per 100,000 inhabitants per year. Mortality in patients with AIM is affected by the time required to diagnose an AIM case and by the time required to commence treatment. A pocket device that would allow simple monitoring of cardiac activity, for example with a single lead, but, if necessary, that would switch to monitoring of up to 21 leads to exclude or confirm a heart attack, is missing on the market. ECG devices measure 12 leads and to detect a heart attack the electrodes are moved intricately. There is not any ECG device for an easy switching from 12 to 21 leads available on the market. Holters or monitors at a hospital bed, detect up to 7 leads. There is no possibility to measure up to 21 leads with a simple switch to eliminate a heart attack. However, there is no pocket cardio first aid kit that persons could have near themselves permanently, especially those at high risk of AIM, so that in the event of a symptom they could make a preliminary diagnosis of AIM and pass the results remotely to medical staff, and if instructed, they could take the appropriate anti-AIM drug from the first aid kit and use a chemical test for AIM, which could significantly speed up the treatment process. A cardio first aid kit could also be used for prevention.

Devices for sensing of multi-lead ECG enabling monitoring of chest leads are not available in a pocket design, they are large, stationary, or they are complicated, using a permanent installation of electrodes on the body that are glued, fixed by clips, strips or suction cups, and the device setup and evaluation are complex, they are designed for medical facilities and they are not suitable for use as a pocket monitoring device carried by the monitored person. There are not any simple pocket devices for short-term sensing of the single-lead ECG heart rate curve for such preliminary diagnosis that would be understandable also to a layperson, for example also to the monitored person, which would allow to this person, in case of finding any arrhythmia, or ST segment elevation, or other deviation from the normal ECG, especially for detection of infarction, to make possible to expand such device for providing of a multi-lead ECG, such as an up to 12-lead ECG, to allow the precise diagnostics, preferably with the assistance of the qualified medical personnel.

With regard to the multiple lead ECGs, there is no such device that could be easily and quickly attached to body during a short-time ECG sensing, just by holding it with fingers, so that the electrodes would not have to be permanently attached to the body, for example by suction cups, wristbands, braces, by gluing, by a belt, or by other mechanical means, that would protract, complicate this ECG measurement, increase its costs, make the installation and uninstallation more complex, and that would require storage of a larger number of bulky parts.

There is no simple device for monitoring of cardiac activity, for example for sensing of the heart rate curve, which could be extended to ECG sensing, or to a device for sensing of less leads, such for a single-lead ECG, that could be converted to a multi-lead ECG device, if necessary, for example, it is necessary to detect deviations from the normal. In most cases, the simple monitoring of cardiac activity is sufficient, and deploying a multi-lead ECG immediately in the beginning would be unnecessary, because the initial detection of any ECG deviations from the normal state, especially of arrhythmia, can usually be detected using the heart rate curve, and the ST segment elevation to detect infarction state using the method described in this application, already by using a single-lead ECG. Deployment of a multi-lead ECG already to make a preliminary diagnosis would be inappropriate due to complexity, energy consumption, larger device size due to the need of a larger battery, and because of time-consuming operation, despite the fact that its operation would be difficult to manage by a layperson, i.e. by a common monitored person.

If necessary, for example to rule out or confirm a heart attack, recording of chest leads, preferably on a multi-lead ECG, is necessary, and should be used immediately at the first symptoms for life-saving. A multi-lead ECG with chest lead recordings that could be used to expand a simple single-lead ECG device or a device for heart rate monitoring and that would be of pocket design at the same time does not exist in the prior art. A pocket embodiment of a multi-lead ECG device for sensing chest leads V1 to V6, which persons exhibiting risk factors might always carry with themselves, and if necessary carry out a multi-lead ECG with chest leads, which ECG they could then transmit from anywhere where they are located at the given moment, for example via the mobile operator's network, for evaluation by medical personnel, such as in an emergency service, or in a monitoring center, is also not existing.

A device that allows easy monitoring of cardiac activity, for example of heart rate curve and/or of a less-lead ECG, but which, if a more detailed diagnosis needed, could be quickly changed to a multi-lead ECG, especially for monitoring of the chest leads, in case of suspected heart attack, is also not existing. A simple pocket model of a device for monitoring cardiac activity, such as a one-lead or two-lead ECG, which would allow to carry out a chest lead ECG and which could be viewed live by the monitored person or by medical staff is also not existing.

A complete ECG recording, i. e. that of 21 leads, is required to fully evaluate the threat or occurrence of a heart attack. For such sensing, at least 10 conductors are required in case of a prior art device, by means of which conductors the signals from the body of the monitored person are brought to the ECG device. Connection of these conductors to the patient's body by means of suction cups, terminals and/or glued probes is uncomfortable and restricts movements, i.e. it is unsuitable for a longer measurement.

### SUMMARY OF THE INVENTION

The shortcomings of the prior art are solved by the device according to the invention. The subject-matter of the present invention is a multi-lead ECG device utilizing a lower-lead monitor, which monitor is switched, to make a possible sensing of a greater number of leads, from the primary electrodes allowing sensing of fewer leads to secondary electrodes allowing sensing of more leads. The lower-lead monitor is connected to electrodes, preferably to primary ones, allowing to sense cardiac signals for processing of data for a certain number of leads by the monitor, which number is limited by the number of electrodes and/or by monitor performance, which performance is given in particular by the number of input amplifiers, the so called frontend. To process a higher number of leads, the monitor is switched over or relocated, for preferably sequential sensing from a higher number of electrodes and/or circuits, preferably of those located in a sensing unit, which electrodes and/or circuits are sequentially switched to the monitor, preferably in sets, via an interconnecting field and/or a movable electrode or electrodes is/are used for sequential sensing of cardiac signals at sites determined for detecting via chest leads.

The multi-lead ECG device comprises:
circuits, preferably for forming of one of the following: a Wilson's terminal, a strengthened Kranz's terminal, a pseudo-Kranz's terminal;
electrodes for sensing of cardiac signals, preferably consisting of at least two primary electrodes interconnected to a monitor and secondary electrodes, to which secondary electrodes the monitor is connected from the primary electrodes, or which secondary electrodes are connected to the primary electrodes;
a sensing unit comprising at least two electrodes, preferably formed by the secondary electrodes, for sensing of cardiac signals and/or circuits, which sensing unit is adapted to be connected to the monitor instead of the primary electrodes;
connecting elements and/or switching over elements for switching between sensing from the primary electrodes and sensing from the secondary electrodes, located in the sensing unit;
a monitor for processing of cardiac signals sensed from at least two primary electrodes into digital data for displaying of at least one ECG lead, adapted to switch over to sensing from the secondary electrodes located in the sensing unit;
a memory for storing of digital data, processed by the monitor, from analog cardiac signals sensed by the base electrodes and/or by the electrodes located in the sensing unit;
communication circuits for wired or wireless transmission of digital data, live or from memory, to at least one cooperating unit, in which unit or on which unit the monitor is located, or to a cooperating unit located remotely from the monitor;
at least one cooperating unit located remotely to the monitor, or a cooperating unit in which or on which the monitor is located, adapted to display ECG curves from the transmitted digital data by the communication circuit live or from memory for sequential or simultaneous displaying.

The electrodes are located on one of the following: a monitor, a cooperating unit in which or on which the monitor is located; a cooperating unit located remotely with regard to the monitor and connected by a cable and a connecting element to the monitor; a sensing unit.

The device includes an interconnecting field, which field connects the electrode sets and/or circuitry of the sensing unit to the monitor sequentially to increase the number of ECG leads, which leads can be obtained from cardiac signals sensed by the electrodes opposite to leads that can be obtained by sensing from the base electrodes, wherein an increase is reached by gradual sensing of cardiac signals from sequentially interconnecting field of electrode sets and or circuits located in the sensing unit for sequential processing by the monitor.

The device according to the invention comprises further one movable electrode for sequential application to the sites intended for sensing the chest leads, for sequential sensing and subsequent processing of cardiac signals by the monitor.

The sensing unit is adapted for sequential relocation of at least one movable electrode to sites determined for sensing of cardiac signals of the chest leads for processing them by the monitor into digital data for displaying of the ECG chest leads.

Preferably, the monitor is connected to electrodes located on the monitor or on the cooperating unit, in which the monitor is located, which electrodes allow one-time sensing of the cardiac signals, and the long-term or permanent sensing is achieved by switching to the electrodes located in the sensing unit, preferably in the shape of a chest belt.

Preferably, the monitor is located separately, or in, or on a cooperating unit, for a one-time or a short-term monitoring by means of sensing from those electrodes, that are remote from the monitor, that are connected by a cable and connecting elements to the monitor, which electrodes are intended to be placed on the patient's body, on chest, arms or fingers. The monitor is adapted for a long-term or permanent monitoring by disconnecting of the connecting element and by interconnecting of another interconnecting element with a cable leading to the electrodes that are located in the sensing unit, preferably in the form of a belt.

Preferably, the monitor is adapted for relocation by means of the connecting elements between the sensing unit with electrodes and the sensing units with a higher number of electrodes than what is the previous number of the electrodes, and for sensing of a larger number of cardiac signals by a higher number and/or more advanced model of electrodes to allow processing of the cardiac signals by the monitor into a higher number of ECG leads than what allow the electrodes, or in the case of a monitor located in a sensor unit with electrodes adapted only for the one-time or the short-term testing by applying electrodes to the body, by relocation of the monitor into the sensor unit with electrodes, preferably in the form of a belt, it is allowed the long-term or permanent sensing.

The device for the multi-lead ECG of the present invention further comprises a memory, which memory is adapted for sequential storing of digital data, which data are processed by the monitor sequentially from the analog cardiac signals sensed from electrodes sequentially connected by the interconnecting field to the monitor, which relocated electrodes are situated in the sensing unit.

The pocket ECG for checking of infarction state according to the invention comprises a sensing unit formed by a chest belt for sensing at least twelve ECG leads. All ECG electrodes that are required for sensing of the twelve ECG leads are located on the chest belt, or a part of them is placed on at least one of the parts for fixing of the electrodes, which electrodes are fixed removable to the chest belt, they form one whole with the chest belt, or the LL electrode is placed on a small board of the electrode connected to the chest belt with a cable.

The pocket monitor of cardiac signals is located on the body or on/in the clothes of the monitored person, it is adapted for simultaneous processing of cardiac signals from at least twelve ECG leads, and this from all ECG leads, which the sensing unit is able to sense; or the monitor is adapted to process simultaneously cardiac signals from a smaller number of leads than what is the it number the sensing unit is adapted to sense, and it is connected to the sensing unit via an interconnecting field, which field connects the monitor at first to that number of electrodes, from which the monitor is adapted to process the cardiac signals, and subsequently, it is sequentially connected to further electrodes of the sensing unit for sequential processing of further cardiac signals, until all of the cardiac signals, which the sensing unit is adapted to sense, are processed.

The processed cardiac signals are transferred from the monitor into at least one of the following: a display; a memory; a communication unit. The display displays the ECG processed by the monitor from the cardiac signals, and the memory is used to store the cardiac signals processed by the monitor, and the communication unit is designed to transmit cardiac signals processed by the monitor to the cooperating units.

The cardiac signals for at least twelve ECG leads are sensed from the ECG electrodes for the lead I, formed by the RA electrode and the LA electrode, for the lead II, formed by the RA electrode and the LL electrode, or by connecting to a pseudo-Kranz's terminal, which terminal substitutes the electrode for LL, and for the chest leads V1 to V6, by electrodes for the leads V1 to V6 opposite to the pseudo-Kranz's terminal. The ECG leads III to IV are calculated from ECG leads I and II.

By connecting of the electrode of the Kranz's terminal via a resistor to the pseudo-Kranz's terminal, it will become a strengthened Kranz's terminal, and by connecting of the LL electrode via a resistor to the pseudo-Kranz's terminal, it will become a Wilson's terminal.

The strengthened Kranz's terminal or the Wilson's terminal replace the pseudo-Kranz's terminal for more accurate sensing. The cardiac signals sensed from the strengthened Kranz's terminal or the Wilson's terminal, providing the electrodes for the chest leads V1 to V6, are used to be processed by the monitor for the V1 to V6 ECG chest leads, and the strengthened Kranz's terminal replaces the LL for sensing cardiac signals against the RA electrode for processing to a two-lead ECG.

The electrode mounting parts consist of at least one of: a board; an electrode segment; rotatable strips; a bridge.

For placing of all electrodes on the chest belt, this belt is formed as a wide chest belt, where the electrodes for V4 to V9 and the electrodes for V4R to V6R are in one plane situated in the lower part of the wide chest belt and the electrodes for V1 to V3 and for V1R to V3R are placed in an ac in the upper part of the belt at the corresponding places for their sensing, wherein, an opening is preferably formed for the nipples, behind which opening the lower part and the upper parts of the belt are reconnected again, the belt is wrapped around the chest, and its ends are connected together by a fixture providing adjustable belt length.

By adding of electrodes for leads V1R to V6R and leads V7 to V9 to electrodes for leads LA, LA, LL, RL and the chest leads V1 to V6 on the chest belt or to the electrode mounting parts, the capacity of the sensor unit is expanded from twelve leads to a maximum of 21 ECG leads.

The chest belt consists of a multi-electrode base with electrodes for RA and LA on the sides of the chest belt or on removable right and left side straps; the electrodes for the chest leads V1 to V3, V1R to V3R are placed on the chest belt, and the LL electrode is replaced by a strengthened Kranz's terminal or a pseudo-Kranz's terminal in case the bridge is not used. If the bridge is used, then the chest leads and the LL electrode are located on the bridge, or alternatively, the LL electrode is located on the left side band or on the board. The bridge is removable, and the left-side electrodes for V4 to V6, right-side electrodes for V4R to V6R and the back electrodes V7 to V9, and the RL are located on the chest belt. Alternatively, the electrode for the RL is placed on the right-side strip below, if it is used.

The chest belt is adapted for permanent attachment to the chest for permanent monitoring or for short-term application, wherein, preferably, it comprises a brace for a comfortable inserting of the chest belt under the shirt on the back, when for example by leaning against the chair backrest the belt is fixed on one side and by leaning of the arm against the extension it is fixed also on the other side for fixing of the chest belt in a stable position relative to the chest to provide for high-quality sensing from the ECG electrodes without any interference caused by any movement of the electrodes against the chest.

The monitor is connectable to the sensor unit by means of connecting elements, what allows to move the monitor between the sensor units.

The interconnecting field comprises mechanical and/or electronic interconnecting elements. The electronic interconnecting elements are controlled by means of a control unit of the interconnection field by means of control elements located on the sensor unit and/or on the monitor or they are controlled from the cooperating units.

The monitor is interconnected by means of the interconnecting field to the sensor unit with ECG electrodes. The single-channel monitor is interconnected by means of the interconnecting field to the electrodes, what allows sequential sensing of individual analog cardiac signals for sensing of up to 15 chest leads, two limb leads I, II, and processing of the analog cardiac signals by the monitor into their digital form of digital data for digital transmission and/or for their displaying.

The dual-channel monitor allows to sense signals for leads I and II simultaneously, and to calculate four leads, III and VF, and VR and VL, from them. It also allows further switching from the sensing of the leads I and II to the chest leads, for sensing of up to 15 chest leads, always of two leads at a time.

The three-channel monitor allows to sense leads I, II and to make a calculation from them of four leads and one chest lead at the same time, wherein, after switching of the interconnecting field from the sensing of the leads I and H to sensing of the chest leads, it is possible to sense up to 15 chest leads by consecutive switching of electrodes from the set of the chest electrodes, always of up to three chest leads at a time.

The eight-channel monitor allows to sense leads I, II and to make a calculation from them of four leads, and further to sense the left-side chest leads V1 to V6 simultaneously for a 12-lead ECG, wherein, for right-side and the back leads, it is possible to switch the respective electrodes by the interconnecting field to the monitor for sensing of up to 17 ECG leads and to calculate another four leads.

The seventeen-channel channel monitor allows to sense all 17 leads simultaneously and to calculate 4 leads for a complete 12-lead ECG, plus 6 right-side chest leads and 3 back chest leads, i.e. for a 21-lead ECG.

### EXPLANATION OF FIGURES IN THE DRAWINGS

FIG. 1 shows the base of a universal monitor for sensing a single-lead ECG using Kranz's terminal by placing it on the chest for sensing V1 to V6, and by placing of fingers for a short-term sensing of the lead I of the ECG;

FIG. 2 shows the construction of a classical Wilson's terminal;

FIG. 3 shows the chest sites recommended for sensing of leads V1 to V6;

FIG. 4 shows the formation of a Wilson's terminal from shifted sensing points;

FIG. 5 shows the formation of the Kranz's terminal area

FIG. 6 shows the location of the base of a universal monitor attached to the chest by a belt for sensing the pseudo-lead V5 by means of a chest belt for long-term sensing;

FIG. 6A shows the location of the monitor base for sensing of the V1 lead;

FIG. 6B shows the location of the monitor base for sensing of the V2 lead;

FIG. 6C shows the location of the monitor base for sensing of the V3 lead;

FIG. 6D shows the location of the monitor base for sensing of the V4 lead;

FIG. 6E shows the location of the monitor base for sensing of the V5 lead;

FIG. 6F shows the location of the monitor base for sensing of the V6 lead;

FIG. 7 shows the location of the base of the universal monitor on a wristband for sensing of the I lead;

FIG. 8 shows a base for sensing of up to a 12-lead ECG by switching of the leads by applying to a chest, held by fingers on electrodes for LA, RA;

FIG. 9 shows the sensing of a multi-lead ECG by applying to the chest and of electrodes for LA, RA, held by fingers;

FIG. 10 shows sensing of a multi-lead ECG by applying of finger-held electrodes, using a board of leg electrodes;

FIG. 11 shows a block diagram of the sensing of leads I, II, V1 to V6;

FIG. 12 shows a table for switching between the measured leads;

FIG. 13 shows the display of ECG, pulse, arrhythmia data and curves;

FIG. 14 shows a conventional 12-lead ECG;

FIG. 15 shows an example of a solution for switching between the sensed leads;

FIG. 16 shows the sensing of two leads at once by a two-channel monitor;

FIG. 17 shows the sensing of two V leads at a time;

FIG. 18 shows a block diagram of a monitor, a monitor base, and an electrode board;

FIG. 19 shows sensing using a two-channel abdominal base and a two-electrode small board;

FIG. 20 shows sensing using a multi-channel base and a two-electrode small board;

FIG. 21 shows a block diagram of a three-channel sensing;

FIG. 22 shows a 12-lead sensing using a chest electrode board;

FIG. 23 shows a block diagram of an eight-channel monitor for 12-lead sensing;

FIG. 24 shows the use of a belt with large collecting electrodes;

FIG. 25 shows the use of a long-term sensing belt with a position for an additional battery;

FIG. 26 shows the use of a secure connecting of an additional battery to a belt monitor;

FIG. 27 shows elements of a kit for sensing 1-12 lead ECG;

FIG. 28 shows a kit of sensing and monitoring units;

FIG. 29 shows an expanded kit of units;

FIG. 30 shows diagnostics of an elevation of the ST segment;

FIG. 31 shows a cardio first aid kit;

FIG. 32 shows the base for RL and its use;

FIG. 33 shows the base for RL and the two Vx leads and the placement of the sub-board for twice Vx;

FIG. 34 shows an extended base, its block diagram, the principle of measuring by sliding it, the use of a reinforcement extending a part of the base, which is of fixed length, or collapsible on the principle of inserting or rotating the segments of the extending part;

FIG. 35 shows the base with a reference and attachment of a small board, the electrode small boards on a cable, the electrode small boards for the two-channel monitor and the small boards of three chest electrodes, as well as the placing of the base and boards on the body of the monitored person;

FIG. 36 shows a block diagram of a connection of the input circuits of a monitor with a reference output;

FIG. 37 shows the sensing of the left-chest and right-chest leads against the Kranz's terminal using a base with reference;

FIG. 38 shows the sensing of the left-chest and right-chest leads against the Kranz's terminal using a reference by means of an RL base with reference;

FIG. 39 shows the sequential sensing of the chest leads using the base with a reference and small boards with an electrode for Vx, small boards for a two-channel monitor and small boards for three chest electrodes;

FIG. 40 shows sensing of the grouped chest signals against each other and against the Kranz's terminal, and this by means of a base for RL and by means of changing the position of the base by shifting, and also it shows a block diagram of sensing and injecting the reference signal;

FIG. 41 shows sensing of the chest leads by means of a complete base, which, after completion by an enlarging board or by electrode segments, can sense all chest leads at the usual width of the chest belt;

FIG. 42 shows the formation of a strengthened Kranz's terminal and a pseudo-Kranz's terminal;

FIG. 43 shows the formation of a Wilson's terminal and a pseudo-Wilson's terminal using side and shoulder electrodes and electrodes on an abdomen board;

FIG. 44 shows a simplified base with an extension on the arm and its use in cooperation with electrode small boards for sensing with a Wilson's or Kranz's terminal, and a block wiring diagram;

FIG. 45 shows a multi-electrode base with transverse strips with electrodes and side strips for full ECG sensing;

FIG. 46 shows a wide chest belt that can sense also chest leads V1 to V3 and V3R;

FIG. 47 shows sensing by means of a sensing unit on a belt with side electrodes, with side strips and with shoulder straps and with an electrode for LL on the abdominal electrode strip or with electrodes for LL and RL on the terminals;

FIG. 48 shows schematically the connection of electrode sets via an interconnecting field to the input circuits of a monitor;

FIG. 49 shows a sensor unit adapted for switching between sensing with a Kranz's terminal for sensing with a Wilson's terminal and a block diagram of the connection of the electrodes to the monitor;

FIG. 50 shows the base with a reference allowing switching from sensing by means of the Kranz's terminal to sensing by means of the Wilson's terminal and a block diagram of the wiring;

FIG. 51 shows the change from sensing with the Kranz's terminal to the sensing with the Wilson's terminal by relocation of the monitor from the base located on the chest to the base for LL, RL, which is located on the abdomen and with using of the electrode small board for the two-channel monitor;

FIG. 52 shows a block diagram of t communication of the monitor and of all cooperating units;

FIG. 53 shows displaying of curves on a display of the evaluation and display unit;

FIG. 54 shows a monitor with electrodes and its use in a holder on a base for applying;

FIG. 55 shows a multi-electrode base with an extension to be placed on an arm and the use of this base for sensing with a strengthened Kranz's terminal, and further a block diagram of connection of the electrodes to the monitor, parts for fixing of the electrodes, a multi-electrode base, and a wide chest belt.

### EXAMPLES OF EMBODIMENTS OF THE INVENTION

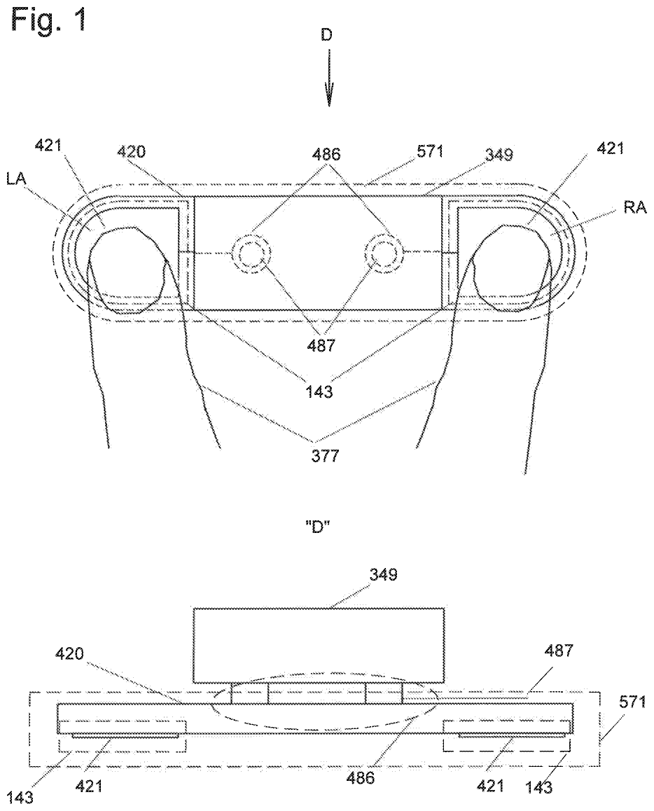

FIG. 1 shows a monitor 349 located on a sensor unit 571, preferably formed by a universal monitor base 420, by means of connecting elements 486, preferably formed by snap fasteners 487, which simultaneously provide electrical contact for two ECG electrodes 143, preferably formed by base electrodes 421 located on the opposite side of the base 420 than that of the monitor 349.

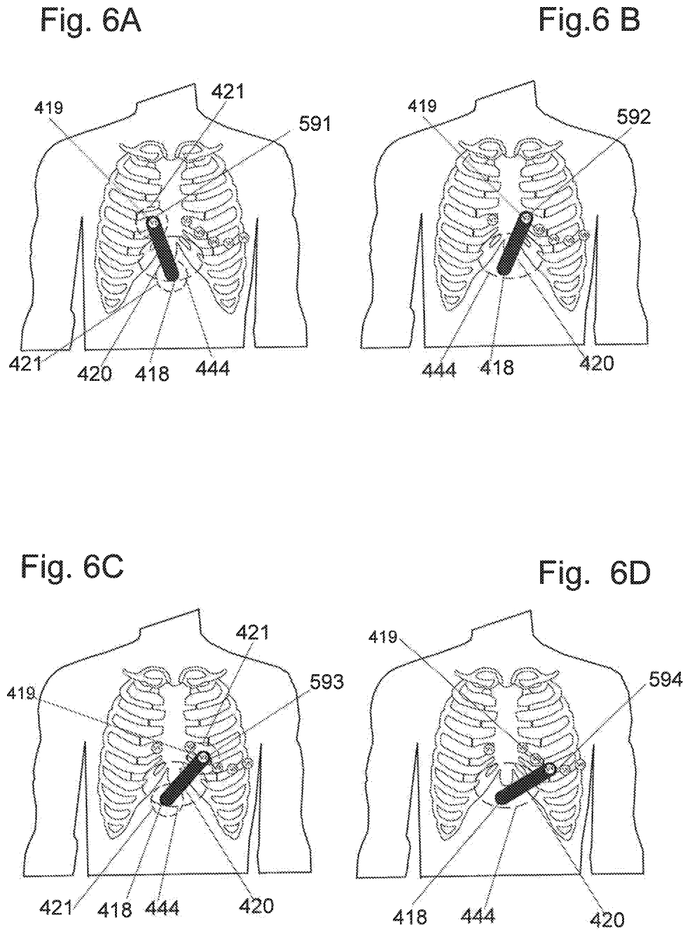

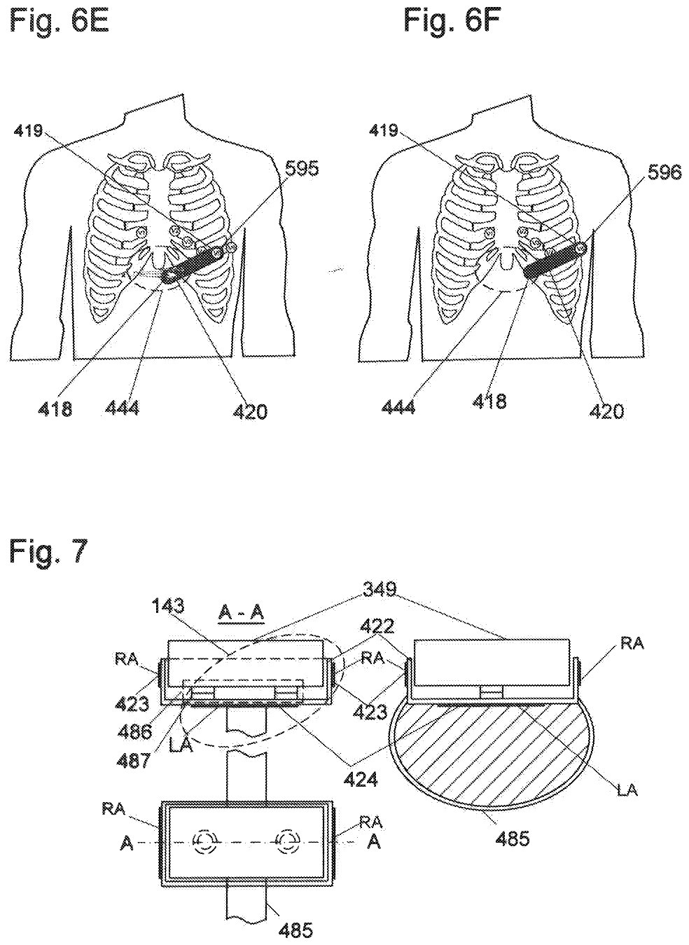

Preferably, the monitor 349 is a single-lead, or a multi-lead one using single lead. If it is placed on the base 420, it is possible to use it to sense the cardiac signal after attaching of the base 420, or of the electrodes 421 of the base, to a suitable place on the body, especially in the chest area, where it senses with one electrode, and preferably in the places for chest leads V1 to V6; with the second electrode it senses in the region 444 of the Kranz's terminal, shown in FIGS. 5 and 6, for processing of one of the chest leads to an ECG, and preferably for finding of the elevation of the ST segment. Or it is sensed by placing fingers on electrodes 421 of the base. The electrodes then provide the function of electrodes for the left arm (LA) and the right arm (RA) for processing to the first ECG curve.

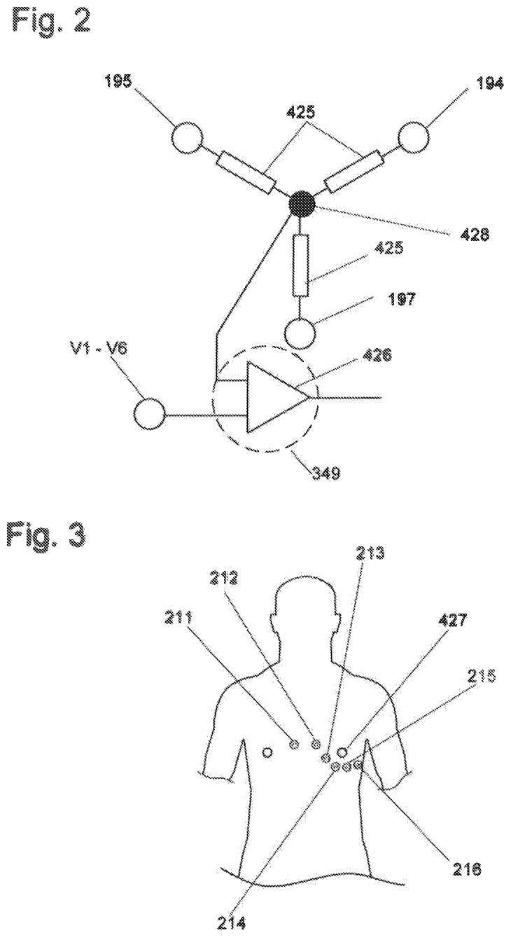

FIG. 2 shows schematically the formation of a Wilson's terminal 428 for sensing of the chest leads V1 to V6.

We create a Wilson's terminal 428 by applying the potentials sensed by the electrodes of the Wilson's terminal formed by the electrodes 194 for RA, 195 for LA, and 197 for LL from the points recommended for sensing, the right arm (RA), the left arm (LA) and the left leg (LL), into a common point through resistors 425 of the same resistance value. Potential of the Wilson's terminal so formed is applied to one of the inputs of a differential operational amplifier 426 contained in the monitor 349, and it is used as a reference for sensing and processing of the chest lead potentials V1 to V6 to a ECG curve. The chest lead electrode 211 is successively attached with one electrode to the sites recommended for the sensing of leads V1 to V6 and their potentials are applied to the other input of the differential operational amplifier 426. In this way, we obtain successively up to 6 of the so-called Wilson's unipolar leads V1 to V6.

FIG. 3 shows the sites on the chest of the subject monitored that are recommended for placement of the electrodes 211 V1 to 216 V6 for sensing of leads V1 to V6. The sites for sensing of the leads V1 and V2 are placed at a level above the nipples, centrally symmetrically around the sternum. The V3 sensing point is at the nipple level closer to the heart. The leads V4 to V6 then continue below the left nipple 427 at approximately the same distance from each other in an arc following the bending of ribs to the left side.

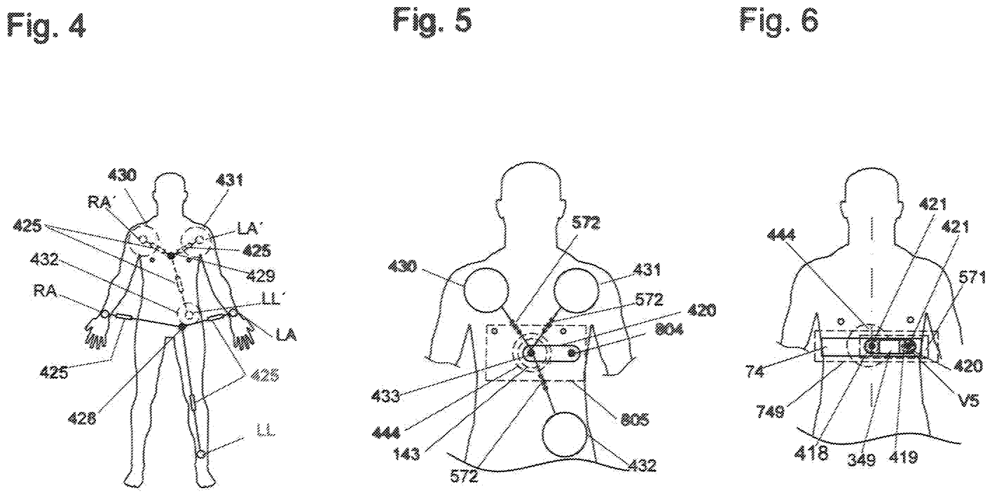

FIG. 4 shows the formation of a Wilson's terminal 429 from the shifted sensing points on the limbs. Standardly, the sensed leads are located on the limbs at sites marked as RA, LA and LL. However, it is possible to sense the corresponding potentials also at other suitable sites, which sites are marked as RA′, LA′ and LL′. The formation of the Wilson's terminal is identical to the procedure as shown in FIG. 2 also for the shifted points for sensing. Again, the potentials are led through identical resistors to a common point, i.e. to the Wilson's terminals 429 for the shifted sensing points.

FIG. 5 shows an electrode 143 formed by an electrode 433 for the Kranz's terminal. The shifted sensing areas of the limbs are referred to as the area 430 for the shifted sensing of the RA′, the area 431 for the shifted sensing of the LA′, and the area 432 for the shifted sensing of the LL′, wherefrom it is possible to sense the cardiac signals for the limbs instead of the classic areas at the end of the limbs, by a sufficiently accurate sensing. If we sense the signal from a human body from the area in the middle between these areas, in the area marked as the area 444 of the Kranz's terminal, we can assume that between this area 444 and the areas for RA′, RL′, LL′ for shifted sensing is approximately the same resistance 572 of the human body. Since, to formation of a Wilson's terminal, identical resistances from these sensed areas for RA′, LA′, LL′ are also used by electrodes located in these areas, it can be assumed that resistances of the human body are created in the area of the Kranz's terminal 444, and thus the chest leads V1 to V6 can be sensed in reference to it, and it was confirmed that the signal in area 444 is the same as the signal with the Wilson's terminal.

The module 805 for sensing the chest leads by means of the Kranz's terminal comprises an electrode 804 for sensing of the chest leads that is located on the chest at a site for sensing of the selected chest lead and an electrode 433 for the Kranz's terminal, which is located in the area 444 of the Kranz's terminal between the abdominal area and the nipples, wherein the electrode 804 for the chest leads and the electrode 433 for the Kranz's terminal are interconnected to the input of the monitor 349 for sensing of the chest leads Vx.

FIG. 6 shows an example of the location of the base 420 of the universal monitor for sensing the pseudo-lead V5.

The figure shows an example of a short-term sensing of the pseudo-chest lead V5 by means of a universal monitor 349 located on the sensing unit 571 held on the chest by fingers or by a chest belt 749, preferably formed by an embracing band 74, preferably formed by the base 420 with ECG electrodes 143, preferably formed by the electrode 421 attached to the area 444 of the Kranz's terminal and by the second electrode 421 instead of the recommended one for the sensing lead V5. This makes possible to sense the V5 pseudo-lead for a short time and similarly also the other chest pseudo-leads. Preferably, for a long-term sensing, the base with the monitor is supplemented with a embracing belt 74 for a long-term attachment to the chest.

FIGS. 6A-6F show sensing of all chest pseudo-leads V1 to V6 against the Kranz's terminal, when the electrode 421 of the base, which is actually used for sensing of the lead V is sequentially attached and held with fingers at the sites recommended for attaching of electrodes for sensing of the respective chest leads, that is at the site 591, which is recommended for sensing of the lead V1, up to the site 596, which is recommended for sensing of the lead V6. The second electrode 421 of the base is always located in the area 444 of the Kranz's terminal, as close as possible to the center.

FIG. 7 shows a universal monitor 349 located on a sensor unit 571, preferably formed by a monitor holder 422 on a wrist, on a wristband 485, into which the monitor 349 can be moved from the base 420 or some other sensing unit 571. This holder 422 is provided with connecting elements 486, preferably formed by snap fasteners 487, which mediate the electrical connection with the ECG electrodes 143 preferably formed by the electrode 423 of the holder and the lower electrode 424 of the holder, which holder forms contact surfaces for sensing arm cardiac signals for RA, LA. The lower electrode 424 of the holder is located on the bottom part of the wristband 485, so that when the wristband is attached to the arm of the monitored person, it automatically realizes electrical contact with the arm on which the monitored person is wearing the wristband. The contact of the other arm is mediated so that the monitored person places the other arm on the electrode 423 of the holder, preferably by grasping the holder in place of the electrodes 423 on the opposite sides of the holder 422 by pressing two fingers against each other. At the same time, it can press the holder, and thus the lower electrode 424 of the holder against the arm, and thus achieve the best possible conductive connection to achieve optimal signal quality. The figure shows placement of the wristband 485 on the left arm, so that the lower electrode 424 of the holder provides conducting of the signal of the left arm (LA) and of the electrode 423 of the holder of the right arm (RA). In the case of fixing the holder 422 on the right arm, the opposite is true.

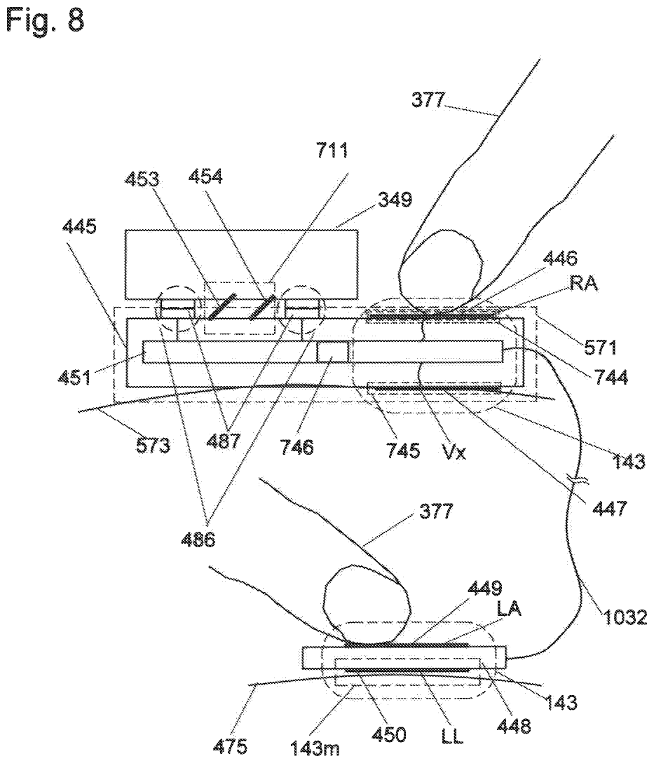

FIG. 8 shows a sensing unit 571 preferably formed by “a monitor base 445 for sensing of a multi-lead ECG”. The monitor base 445 is adapted for placing of a finger 337, preferably of one of the right arm, on the ECG electrode 143, preferably formed by an electrode 744 for fingers, in this example it is formed by “an electrode 446 of a holder for a finger”, for the RA, whereby the holder is pressed by its other, the lower ECG electrode 143, preferably formed by a movable electrode 143m, preferably formed by an electrode 745 to be put on the body, in this example it is formed by “the bottom electrode 447 of a holder for sensing of a multi-lead ECG” in the location recommended for sensing of leads V1 to V6 on the chest 573, in this case sequentially. This applies also to the electrode 446 for a finger, which is situated on a board or on a base, as they are shown in any of the following figures, as well as to the electrode of the small board 449 of two electrodes for a finger. Preferably, the sensing unit 571 is formed “by a small board 448 of two electrodes”, wherein it comprises “an electrode 449 of a small board of two electrodes for a finger” for LA and “the electrode 450 of the second small board of two electrodes” which is attached to the abdomen 475 for sensing of the left leg LL potential. “The small board 450 of two electrodes” is interconnected by a cable 1032 with the base 445 and with the in it comprised “board 451 of electronics of the holder for multi-lead ECG”, to which it transmits the LA, LL signals. The electronics 746, located on the base 445, makes possible to sense the leads V1 to V6, V1R to V6R and V7 to V9 subsequently. The RA signal is sensed by the base electrode 446. All necessary signals LA, RA, LL and the chest leads Vx for up to 19-lead ECG are sensed by said electrodes. The “electronics board 451 of the holder for the multi-lead ECG” is adapted for connecting always of two potentials of the electrodes 446, 447, 449, and 450 to the monitor 349, and this by means of an interconnecting field 711, preferably formed by switches 453 of leads I, II/Vx, and switches 454 for the LL/RA leads.

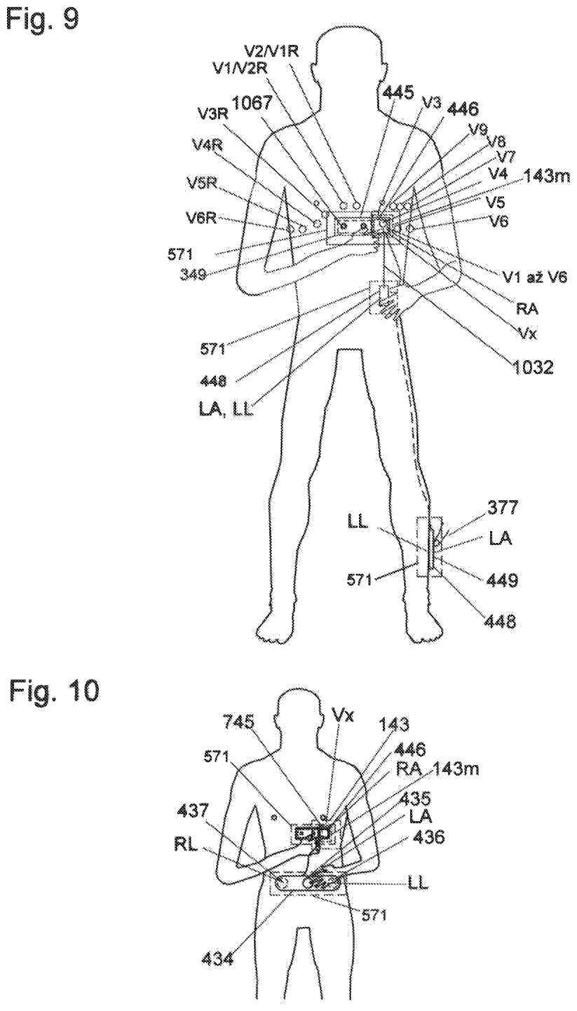

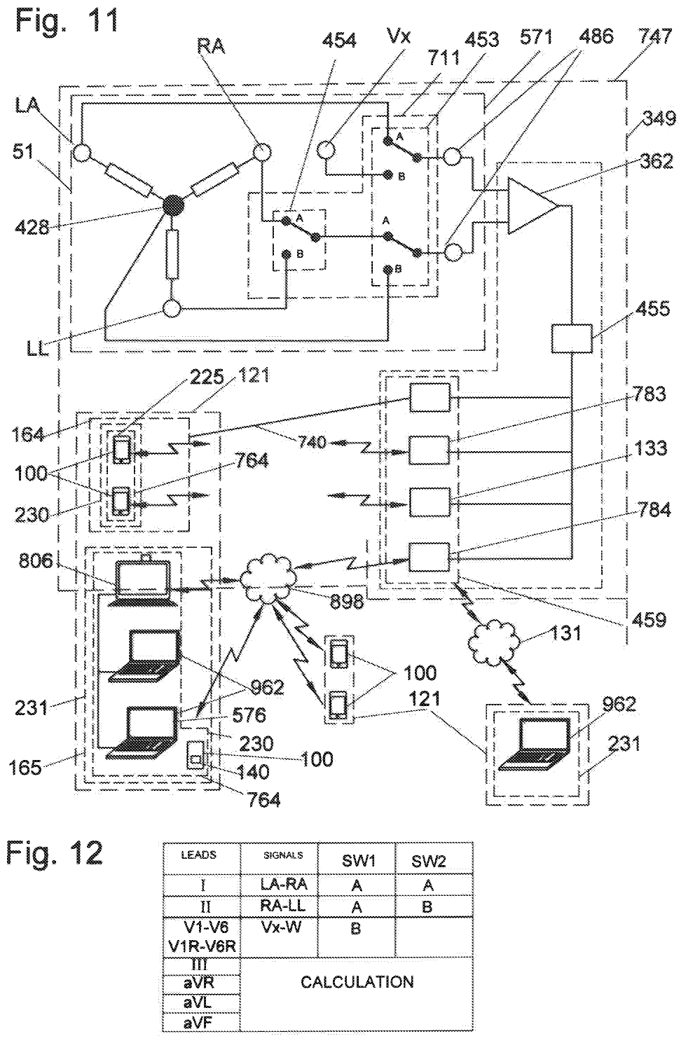

To evaluate the cardiac signals, a single-channel monitor is preferably used, which is based on the principle shown in the block diagram of FIG. 11. The location of the sensing unit 571 on the body by holding of the board is shown in FIG. 9.

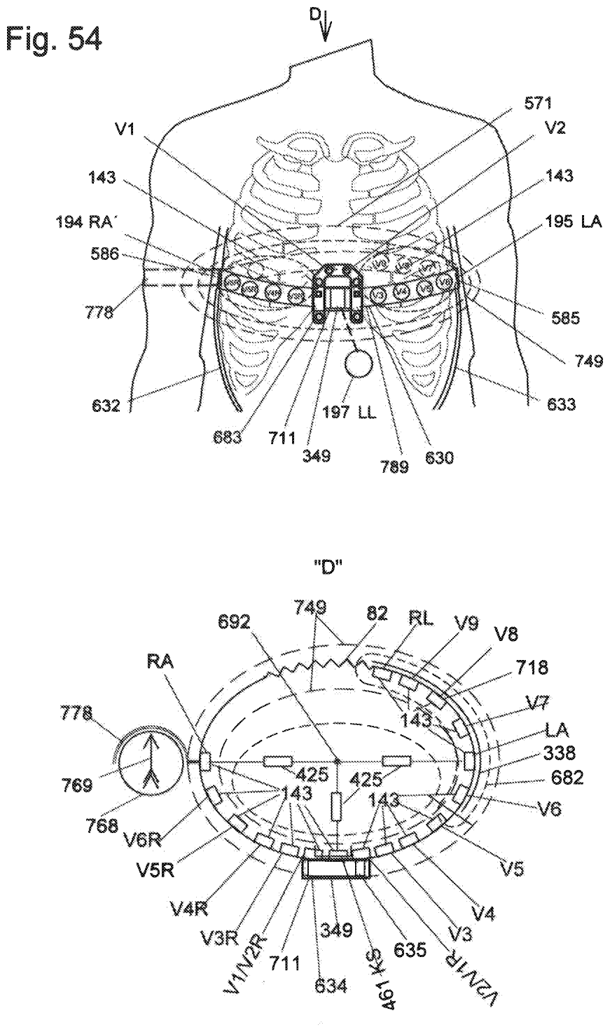

Preferably, the monitor 349 is formed by a monitor 770 with electrodes shown in FIG. 54, which monitor is connected by means of the base 774.

FIG. 9 shows sensing of multi-lead ECG by applying of a sensing unit 571 to the body with finger-held ECG electrodes 143, which unit is preferably formed by a “monitor base 445 for multi-lead sensing of ECG”, which base is held in the right arm, and with another sensing unit 571, preferably formed by a “two-electrode small board 448”, which unit is held with the left arm. In the position of arms shown in this Figure, the leads I (RA-LA) are sensed, as well as voltage course between a finger of the right arm RA, which is sensed by the “electrode 446 of the holder for sensing of the multi-lead ECG for finger” and a finger of the left arm (LA), which is sensed by the “electrode 449 of the two electrode small boards for finger”, further the lead II (RA-LL), as well as the voltage course between the finger of the right arm RA which is sensed by the electrode 446 and a point in the left abdominal area, that is in the area, which is suitable for placing of the LL electrode, which voltage course is sensed by the “electrode 450 of the other small board with two electrodes”. The leads V1-V6 are sensed using gradual relocation of the “base 445 of the monitor for sensing multi-lead ECG” so that the body electrode 745 is sequentially placed in the chest lead sensing sites Vx. By this relocation of the “monitor base 445 doe multi-lead ECG” into positions for sensing of leads V1-V6, a multi-lead ECG is achieved. By further relocation of the base 445, it is possible to sense the right-side chest leads V1R to V6R and the back leads V7 to V9.

The “small board 448 with two electrodes” pressed against the body by the pressure of a finger of the left arm is alternatively attached to the left leg, what is practical for persons wearing sportswear or a skirt.

The reference from the right leg RL is not scanned, and is created artificially using resistors in the monitor 349 or in the sensor unit 571.

FIG. 10 shows applying of a sensing unit 571, preferably formed by a “leg electrode board 434 provided with three electrodes” in the abdomen area to obtain the RL reference, which reference is alternatively used instead of the two-electrode small board 448. The “three-electrode leg electrode board 434” preferably comprises an electrode 435 of the board for leg electrodes with three electrodes for the left arm (LA) and an “electrode 436 of the board for leg electrodes with three electrodes for the left leg” (LL) on the abdomen side, and an “electrode 437 of the board for leg electrodes with three electrodes for the right leg” (RL). This electrode is opposite to the board 448 and serves to obtain the RL lead, which serves as the reference one.

FIG. 11 shows a block diagram of the interconnecting field 711 of a pocket monitor for sensing leads I, II, and V1 to V6 by a sensing unit 571, preferably formed by the base 445 from FIG. 8, and transmitting of signals processed by the monitor 349 to cooperating units 121 and to selected subscribers. By using of the two-electrode small board 448 of FIG. 8 for LA and LL or of the board 434 of leg electrodes of FIG. 10 with three electrodes also for RL and by sequential applying of the “monitor base 445 for multi-lead sensing of ECG” for RA and V1 to V6 and V1R to V6R and V7 to V9, as shown in FIG. 19, and by switching of the switch 453 of the leads I, I/Vx and 454 LL/RA, the signals LA, RA and LL are connected to the front-end 362, whereby sensing of leads I, II is reached. _Further 4 leads III, aVR, aVF, aVL, used to obtain up to a 19-lead ECG, are calculated from the leads I, 11, whereby for calculation pulses of the lead I and pulses of the lead II having the same length R-R are selected. The control unit 455 can process and transmit a complete ECG via the transmission module 459 to the cooperating units 121, namely to the cooperating units 164 that are nearby and/or for example for medical staff to the remote cooperating units 165, consisting of desktop units 231, preferably formed by a server 806, PC 962 and pocket units, preferably formed by a mobile phone 100. For connection to the cooperating units 164, preferably, the transmission module 459 comprises a Wi-Fi unit 783, a Bluetooth module 133, a Zigbee module, and for connection to the cooperating units 165, it comprises a “mobile network transmission module 784” of a “mobile operator's network 898”.

Preferably, the monitor 349 is formed by the monitor 770 with electrodes, which is shown in FIG. 54, which is connected by any adapter of the base 777.

FIG. 12 shows a table of positions of switches of the “monitor base 445 for multi-lead ECG sensing” for sensing of individual leads. The first column indicates names of the leads to be scanned, the second column indicates the recommended points for sensing these leads, among which it is sensed, the third column indicates positions of the switch 453 of the leads I, II/Vx for sensing of the desired lead, and the fourth column indicates the position of the switch 454 for leads LIRA for sensing the desired lead. The leads III, aVR, aVL and aVF are calculated. When sensing the leads V1 to V6, the switch 454 of leads LL/RA is not attached, and the electrode Vx, which is preferably formed by the “electrode 447 of the holder for sensing of the multi-lead ECG lower” for sensing of V1 to V6, is moved sequentially to the points recommended for sensing of V1 to V6. The reference from the RL is obtained by moving the board 434, by which the RL is scanned, or when using the small board 448, the RL reference is formed artificially by resistors in the monitor 349 or in the sensing unit 572.

FIG. 13 shows an example of pocket units 168, which unit is carried by a monitored person, which unit is preferably represented by the unit 764, preferably formed by a mobile phone 100. This example shows ECG, heart rate and arrhythmia curves, which are displayed on the display 225, wherein they are obtained by processing of the measured data in a control unit 455. In the windows 204 of values and information in the upper part numerical values and heart rate 169 are indicated, as well as the variability 170, arrhythmia 171, ST segment elevation 172, and alarms together with a description 167 of the cause of the actual alarm.

When the adjustable limits are exceeded, preferably, a warning signal is triggered, which, if not reset by the monitored person, turns into an alarm, preferably transmitted also to remote cooperating units, preferably to a server 806, and to selected subscribers. In this example, the small display 225 size of the pocket units is taken into account, what allows only a gradual or partial display of a 12-lead ECG. A two-lead ECG, upper, is shown, i.e. the “curve 438 for the lead I” and the lower one is the “curve 439 of the lead V5”. Optionally, the selected leads of any sensed lead are displayed.

The next curve is the heart rate curve 6 together with the regular heart rate limit curves 17. The heart rate curve is produced as a discontinuous curve, connecting points that are created by plotting the values of the recalculated heart rate between adjacent beats, i.e. between the moments of the QRS complex occurrence, they are plotted on the y-axis at the respective time of QRS complex occurrence, with time data 209 on x-axis. The two, the regular heart rate limit curves 17, the upper one and the lower one, define a band for the rhythmic pulses around the heart rate curve, whereby, preferably they are formed ±13% off the average heart rate value, preferably, they are calculated from the fifteen previous heart pulses. A pulse that is too distant from the previous one or too close to the previous one, which is evaluated as the arrhythmic one according to these criteria, deviates from this band, what is visible at first glance. The percentage of arrhythmic pulses out of the total number of pulses is the arrhythmia value 166 shown in the top part of the display. If it exceeds the set value, preferably, an alarm is triggered together with the description 167, in which also the type of arrhythmia, preferably the atrial fibrillation, the flutter, the tachycardia are indicated.

Variability is indicated by a number determining the degree of heart rate variability. A value of elevation of the ST segment 172 or of depression of the ST segment indicate the diagnosis of AIM, and at an adjustable value, an alarm is triggered together with description 167. The alarm is triggered also by other signs of infarction such as in case of a reverse T wave or in other pathological cardiac activity conditions. The fourth curve is the curve 12 of frequency of occurrence of arrhythmic pulses, which clearly expresses density of the frequency of occurrence of arrhythmic pulses and the degree of their deviation from the average heart rate of leads I, II, V1 to V6, wherein, the leads III, aVF, aVL and aVR are calculated.

Due to the small display 225 of the pocket units, the selected leads are shown sequentially. In FIG. 13, leads I and lead V5 are shown, and sequentially, other selected leads can be selected.

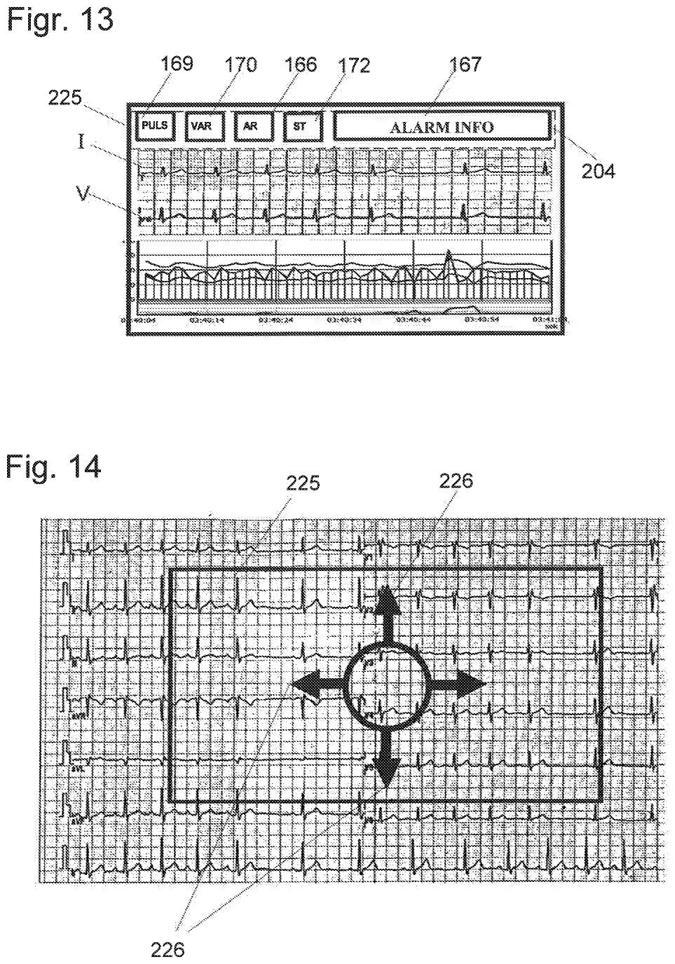

FIG. 14 shows an example of an image on the display 225 of the pocket units 230 of a complete 12-lead ECG, which is obtained by sequential sensing of leads. A section 233 of the display is shown which, by shifting of the ECG in the direction of arrows 254 of the shift of the ECG displays sequentially the entire ECG. Preferably, this shifting is realized by a touch screen display by means of a finger placed in the circle and shifting the ECG in the direction of the arrows as needed.

Therefore, ultimately, the monitored person has an opportunity to see a 12-lead ECG recording so as it is usual in the cardiology centers, but to choose their own imaging scale and to choose for example the desired point in the recording of the desired lead.

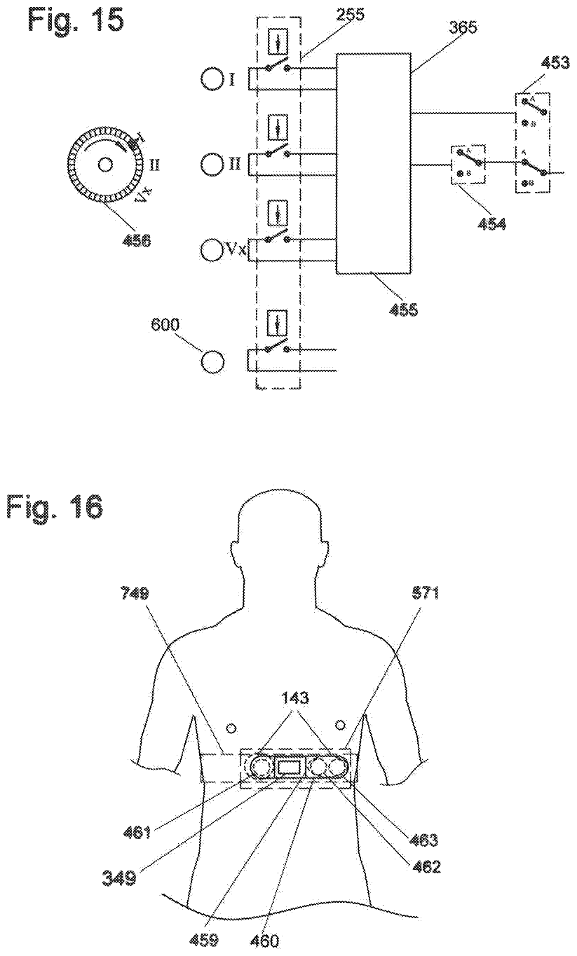

FIG. 15 shows a multifunctional switch 456 of leads, preferably a rotatable one and shaped so that each of its positions is indicated by the respective lead and unambiguously defined by an orientation element on the switch, preferably by a protrusion, which protrusion facilitates the monitored person to switch over the leads correctly. This switch controls both the switch 453 of the leads I, II/Vx and the switch 454 of the leads LL/RA, see example shown in FIG. 11. Alternatively, the monitored person takes over the task of switching of leads by the button 255. Therefore, so the monitored person has also the tactile orientation to which lead it is switched over. It is about the leads I, II, and Vx (x=1 to 6). For each position, the monitored person moves the base 445 of the monitor for a multi-lead ECG sensing with the appropriate electrode to the appropriate body part. Once the monitored person switches the multifunction switch 439 of leads to the appropriate position and places the “base 445 of the monitor for multi-lead ECG” to the appropriate sites, the person presses the pushbutton 600 Start to begin sensing for displaying and/or recording in memory. Alternatively, the task of switching is taken over by the pushbuttons.

FIG. 16 shows a monitor 349, preferably formed by a dual-channel monitor 459, which is located on a sensing unit 571, preferably formed by a “base 460 of the dual-channel monitor”. The base 460 comprises three ECG electrodes 143 located underneath, preferably it is formed by an electrode 461 for sensing of potential of a Kranz's terminal, an electrode 462 for sensing of the lead V4, and an electrode 463 for sensing of the lead V5, wherein, their assembly is placed on the chest in the position shown in this Figure, what allows sensing of two chest leads at once, namely of the leads V4 and 5V.

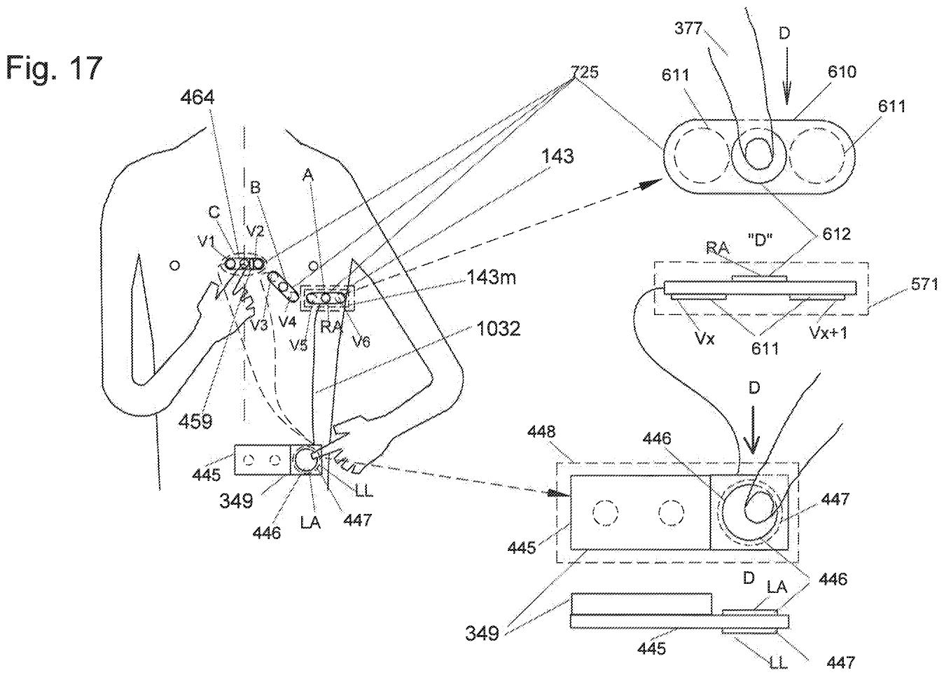

FIG. 17 shows placement of the monitor 349, which is preferably formed by a dual-channel monitor 459 on a sensing unit 571, which is preferably formed by a “base 445 of monitor for multi-lead sensing” and its use. The sensing unit 571, preferably formed by the base 445, is provided with two electrodes 143 for ECG, which are preferably formed with a “base electrode 447 for sensing of multi-lead ECG bottom” for sensing of LL, and with electrode 446 of the base for sensing of the “multi-lead ECG for finger” for sensing of the LA lead.

To the monitor is connected by a cable also the “small board 610 of three electrodes” with “electrode 612 of small board for three electrodes” for finger” for sensing of RA and two “electrodes 611 of small board for three-electrodes, bottom” for sensing of Vx and Vx+1.

The signals from electrodes 612 (RA), 446 (LA) and 447 (LL) are used for formation of potential of the Wilson's terminal, against which potential the chest leads are sensed, i.e. the Vx signals, which are sensed by two electrodes 447 on the underside of the base of the small board 610. “In the view “D” the side view is shown.

The small board 610 of three electrodes is held by a finger of the right arm in the place of electrode 612 for sensing of RA, wherein, the electrodes 611 in the position A sense the leads V5, V6, in the position B the leads V3, V4, and in the position C leads V1 and V2. The base 445 on which the monitor 349 is connected, is pressed with a finger at the location of the electrode 446 for the LA, and of the electrode 447 on the abdominal area for the LL.

FIG. 17 shows further the sequential application of the “base 464 of the dual-channel monitor for finger” with the attached monitor 459 to sites on chest that are recommended for sensing of leads V1 to V6, and this always of two of them, that is of the first sensing V1 and V2, the second sensing V3 and V4, and the third sensing V5 and V6. Simultaneously there are sensed always with the pair of leads Vx and the potential of the right arm RA also the potentials LL and LA by means of a “small board 448 of two electrodes”.

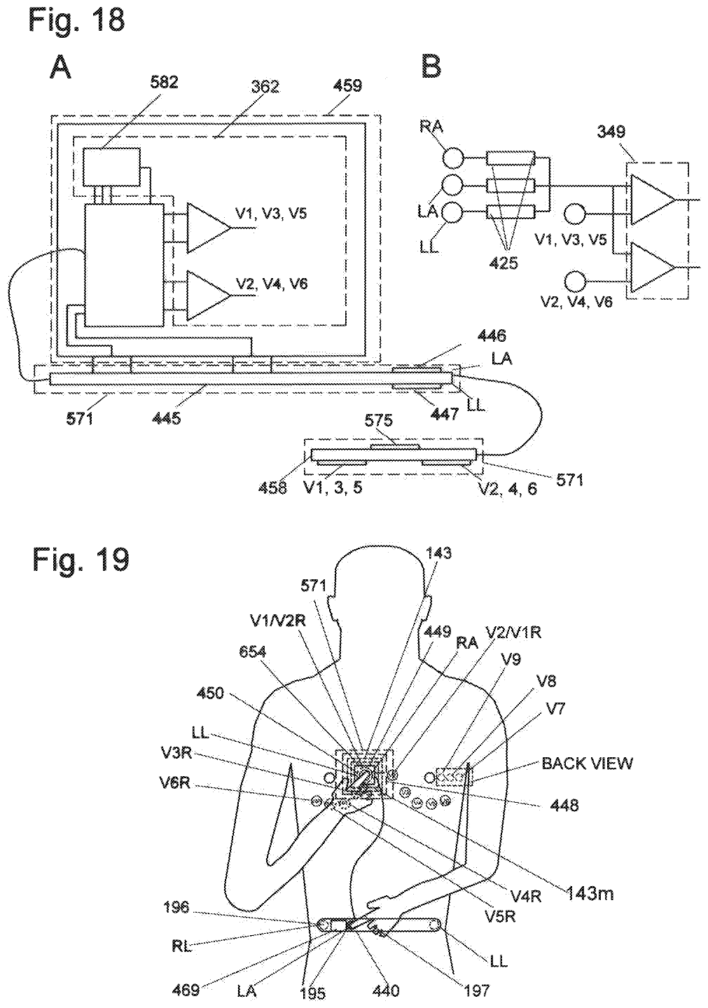

FIG. 18 shows a block diagram A of a two-channel monitor 459 cooperating with a “multi-lead sensing base 445” attached to the abdomen with a left hand finger and a “three-electrode board 458, chest” attached to the chest for the arrangement shown in FIG. 17. Using this configuration, at first, the leads I, II are sensed, and then always pairs of leads Vx and Vx+1. Since these leads are sensed in reference to the Wilson's terminal, to determine their potentials, potentials of the respective electrodes, attached to the recommended sensing sites, are also sensed at the same time, namely the “chest electrode 575 of the three electrode board, the chest one, the for finger one” for RA, the electrode 446 for LA, and the electrode 447 for LL. All signals are transmitted, preferably via a switching field 574, to the front-end input circuits 362 in the monitor 349, where a reference Wilson's terminal is formed from potentials LA, RA and LL in the Wilson's reference circuit 582 and the leads V1 to V6 are evaluated against it. The block diagram B shows the circuits with the formation of the Wilson's terminal from resistors 425 outside the monitor 349.

FIG. 19 shows sensing of a multi-channel ECG by means of a three-channel monitor, which monitor is located on the “base 440 of the abdominal monitor” and of the “electrode small board 654 on the cable”. The “base 440 of the abdominal monitor” is similar to the “board 434 of the leg electrodes with three electrodes”, but additionally, it is adapted for mounting and connecting of a three-channel monitor 469, and allows interconnecting by a cable to other sensing units, i.e. to bases or electrode boards.

In the embodiment shown in the figure, the monitored person holds with his/her left arm, preferably with the index finger, the “base 440 of the abdominal monitor”, what makes possible to sense potentials from the RL, LL and LA sites. With the right arm, the monitored person places to sites for sensing of the chest leads the “two-electrode board 448”, which board comprises the “electrode 450 of the two-electrode board” a on the side which is attached to the chest, which electrode senses the respective V lead, and one electrode on the side turned off the chest, what is the “electrode 449 of the two-electrode board for a finger”, which electrode senses the RA. The sensor unit 571, which is equipped so with a three-channel monitor, is adapted for simultaneous sensing of the leads I, II and of one lead of the chest Vx, wherein, 4 leads are calculated for displaying, namely III, aVF. aVL and aVR. Additional leads Vx are obtained by sequentially relocation of the two electrode board 448 to the points recommended for sensing of individual leads V1 to V6 for 12-lead sensing, as it is shown also in the block diagram in FIG. 21. Reference RL is used for reference.

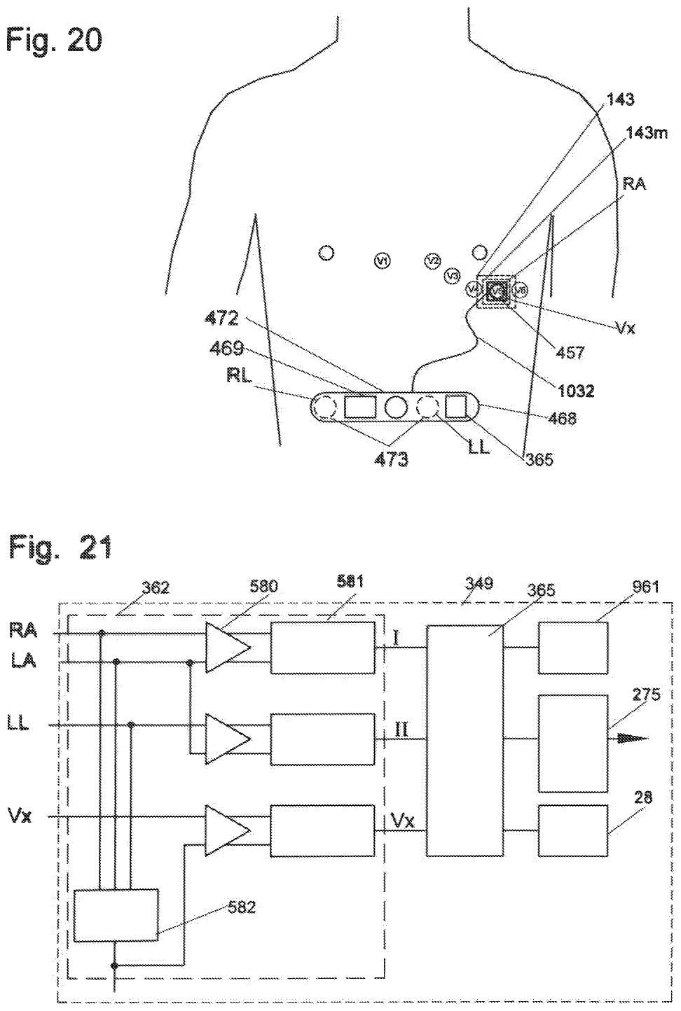

FIG. 20 shows sequential sensing of a 12-lead ECG. A “multi-channel base 468” is used, which is attached to the abdomen and is held by the finger of the left arm in place of the “electrode 472 of the multi-channel finger base”. The potential of the right arm RA is sensed from it. Two “electrodes 473 of the multichannel base, lower” are included in the abdomen area for sensing the RL and LL potentials. The chest leads V1 to V6 are sensed by means of a “two-electrode board 457, chest”, which at the same time senses the RA signal from the finger of the right arm, by which the “two-electrode board 457” is pressed against the chest. At the same time, it is possible to read the leads I, 11 and calculate III, aVL, aVF, aVR and to read the chest leads by gradually moving the board 457.

FIG. 21 shows a block diagram of a 12-lead ECG sensing using a monitor 349, preferably a three-channel monitor 469 (not shown) of FIG. 20. The leads I and H are always sensed, leads Vx, i.e. V1 to V6 sequentially by relocation of the “small board 457 with two electrodes” (not shown) to the points recommended for sensing of leads V1 to V6. Potentials from the places RA, LA, LL, and V1 to Vx are applied to front-end inputs 362. From the potentials RA, LA, LL, a reference for chest leads V1 to V6 is processed in the circuits 582 of the Wilson's terminal. The “monitor control unit 365” processes the signals. This unit calculates leads III, aVF, aVR, and aVF and produces a 12-channel ECG using some selected method. This unit stores the result in the memory 961 and/or it displays it on the display 28 and/or it sends it to the cooperating units by means of the communication unit 275.

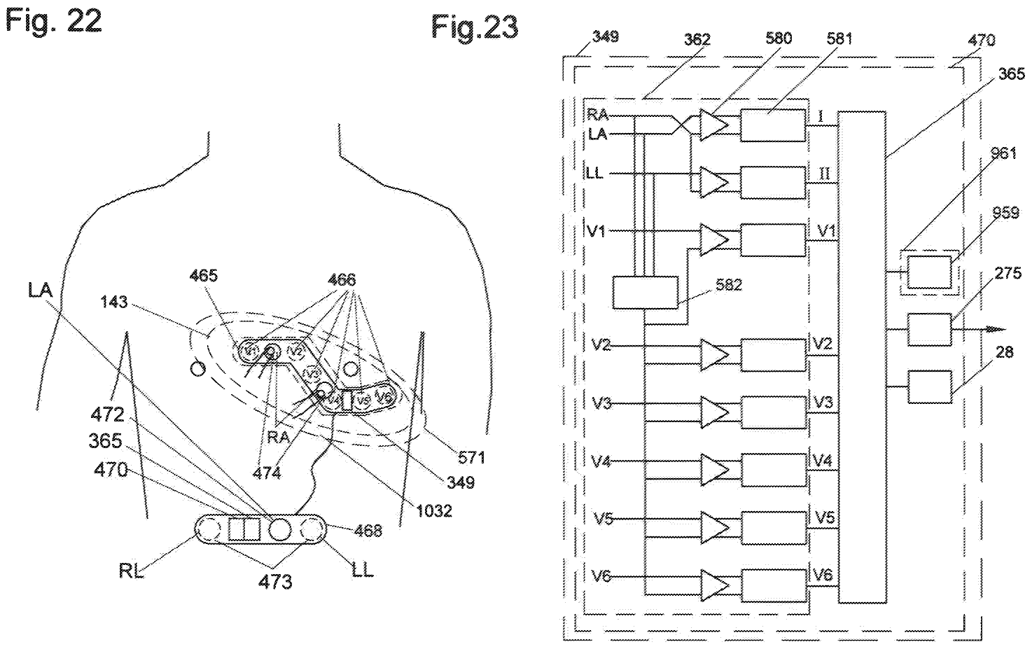

FIG. 22 shows sensing of a 12-lead ECG at one time by means of a sensing unit 571, preferably formed by a “chest electrode board 465”. This board is shaped into a curve so that the six EG electrodes 143, preferably formed by the “electrodes 466 of the chest electrode board for sensing chest leads, located on the side to be attached to the chest, touch the chest at the sites recommended for sensing of leads V1 to V6. Further, the board 466 is provided with ECG electrodes 143 on top side, preferably formed by “two interconnected electrodes 474” for sensing of the right arm signal RA from the fingers 377 and this at sites that are suitable for pressing the board 466 with two fingers against the chest. The board 465 is connected by a cable 1032 to a sensing unit 571, preferably formed with a “multi-channel base 468,” which base is equipped with a monitor 349 provided with the required number of channels that are needed for simultaneous sensing of the planned number of ECG leads. The “multi-channel base 468” is pressed with fingers of the right hand against the abdomen area. On the side turned to abdomen, it is provided with two ECG electrodes 143, preferably formed by “multi-channel base electrodes 473, bottom,” for sensing in the points LL and RL. In case that the base 473 is provided with an eight-channel monitor 470, this gives the required number of channels for leads I, II, V1 to V6 that are sensed, and the other leads 11, aVL, aVR and aVF are calculated.

FIG. 23 shows a block diagram of connection of a monitor 349, preferably formed by an eight-channel monitor 470. If compared to the three-channel monitor 469, this monitor is extended by 5 channels, which channels allow simultaneous sensing of all left-chest side leads and of the leads I and II. Processing of the required 8 channels is provided by the control unit 365 on a twelve-lead ECG for storage in memory 961 and/or displaying on a display 28 or transmitting by means of communication unit 275 to the cooperating units 121 and/or to selected subscribers.

Preferably, the memory 961 is removable, preferably formed by an SD card 959, which card allows relocation into the cooperating data transfer units 121.

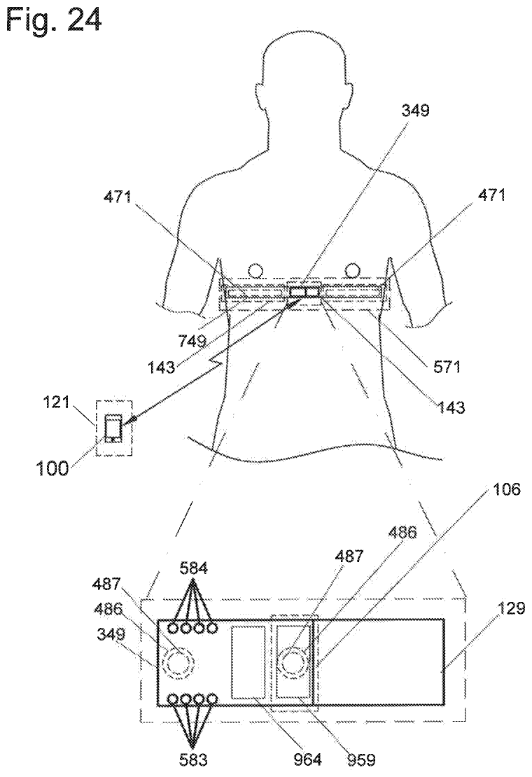

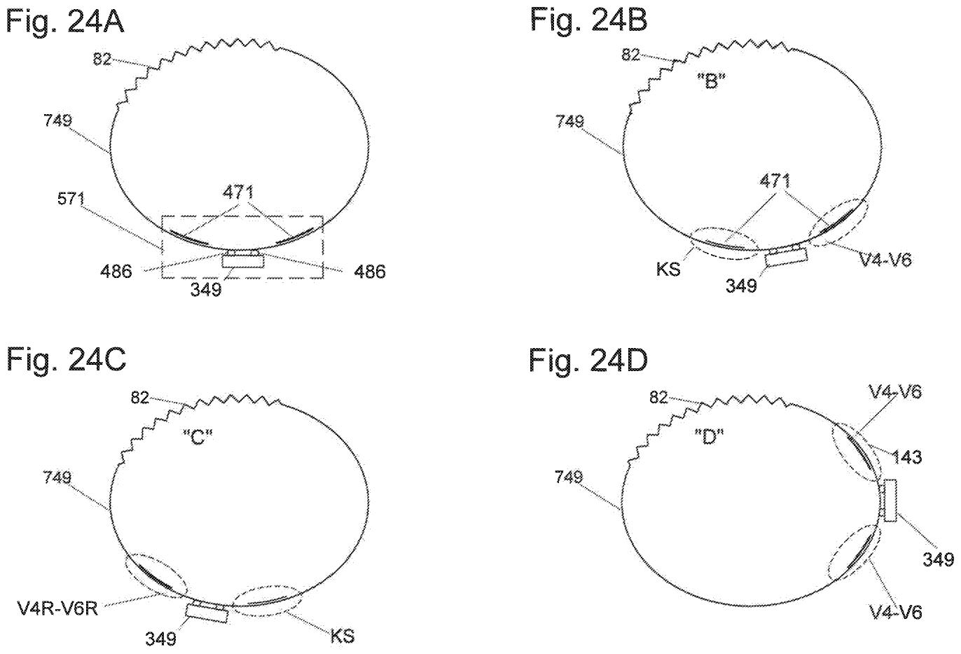

FIG. 24 shows sensing of a single-lead ECG by means of a single-channel monitor 349 mounted on a sensing unit 571, preferably formed by the chest belt 749, by means of attachment elements 486, preferably formed by snap fasteners 487, what allows to move the monitor 349 between the sensing units 571. Preferably, the chest belt 749 is used instead of the base 420 and it is held on the body of the monitored person by hands for a short-term test or by an elastic portion 82 of a belt for the long-term monitoring, which has the advantage consisting in that the chest belt 749, from which the monitor can be detached, is flexible throughout its whole length, is more comfortable to wear, and it is washable, if compared to the base 420, it is made of a strong material, and is better to wear. Preferably, the ECG electrodes 143 are used for monitoring; preferably, they are formed by large-area electrodes 471, which are clustered to sense a plurality of grouped leads.

In the position “A”, the chest belt 749 with two ECG electrodes 143 is attached symmetrically to the sternum and scans the grouped chest leads V4 to V6, and V4R to V6R. Preferably, these leads are used to overview detection of the elevation ST in the left and the right sides of the chest and for detection for example of arrhythmia, for pulse omissions, extra systoles, and for other cardiac activity data that are measurable from the chest by a single-channel monitor 349.

By relocation to the “B” position, the grouped chest leads are sensed from one electrode 143 against the Kranz's terminal, wherein, the other electrode 143 is located in the region 444 of the Kranz's terminal.

In the position “C”, the grouped chest signals of leads V4R to V6R are sensed against the Kranz's terminal.

In the position “D”, the grouped signals V7 to V9 are sensed against the signals V4 to V6 for approximate finding of the elevation of the ST segment and of the leads V7 to V9. Preferably, the monitor 349 is provided with cables 583 for a “marker” to indicate the cardiac signal for sensing at the positions “A” to “D”, and preferably, it is provided with pushbuttons 584 for selecting of the display on the sensed cardiac signals.

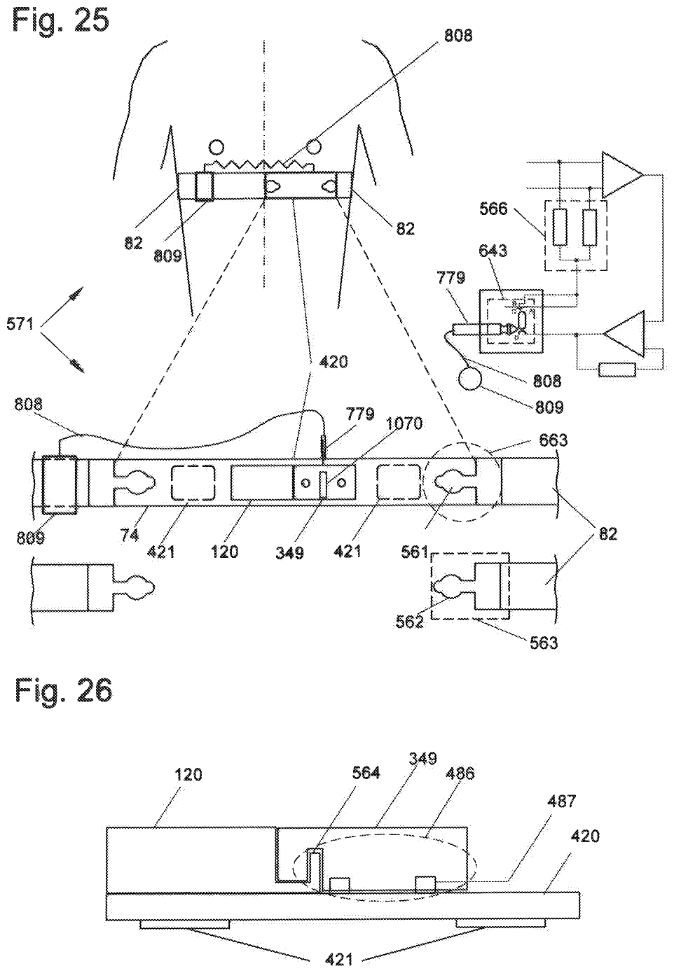

In general, it is valid that the filter which stabilizes more the ECG waveform is more interfering into the ECG curve and distorts it more. Therefore, to determine the elevation of the ST segment, such filter is selected that distorts the ECG curve as little as possible, but sufficiently stabilizes the ECG curve. Better results, when compared to the pseudo-reference, are obtained by using the reference for leads, which reference is preferably removable and it is connected by the reference connector 643, see FIG. 25. Instead of pushbuttons 583, 584, markers and filters are preferably controlled remotely from cooperating units 121, preferably formed by a mobile phone 100. The chest belt is further provided with an additional battery 129, which can be connected to extend the continuous function for a long-term monitoring of the monitor provided with a battery for the short-term monitoring. Further, the monitor is provided with a removable storage medium 106, preferably formed by a SD card 959, in which the ECG recording is preferably stored, preferably throughout the whole time period of sensing, especially if it is not forwarded to the units 121. Preferably, the ECG recording is transferred to the cooperating units 121 by relocation of the storage medium 106. The monitor 349 communicates wirelessly, by means of a communication unit 275, preferably via a Bluetooth, with the cooperating units 121, preferably realized as pocket units 230, preferably as a mobile phone 100 with installed SW allowing to monitor and evaluate the signal received from the monitor 349. It is preferable, when the monitored person during monitoring of arrhythmia and of other data, followed from the pulse, follows the cardiac activity characteristics on data that are preferably displayed on the cooperating and local units 164, which data are processed from the heart rate and its changes only, and from their change in the heart rate curve with regular heart rate limits, and only if it is necessary, especially when arrhythmia is indicated, it is switched over to following of the ECG curve to verify the diagnosis, what is more energy-intensive, and therefore, considering the monitor battery life, it is advantageous to activate it only when needed. At the same time, it is possible to carry out preventive checks of the AIM symptoms by means of the ECG periodically, or only in case of subjective problems. The Monitor 349 is adapted for internal pseudo-reference. The reference carried out by means of the reference electrode is shown in FIG. 25.

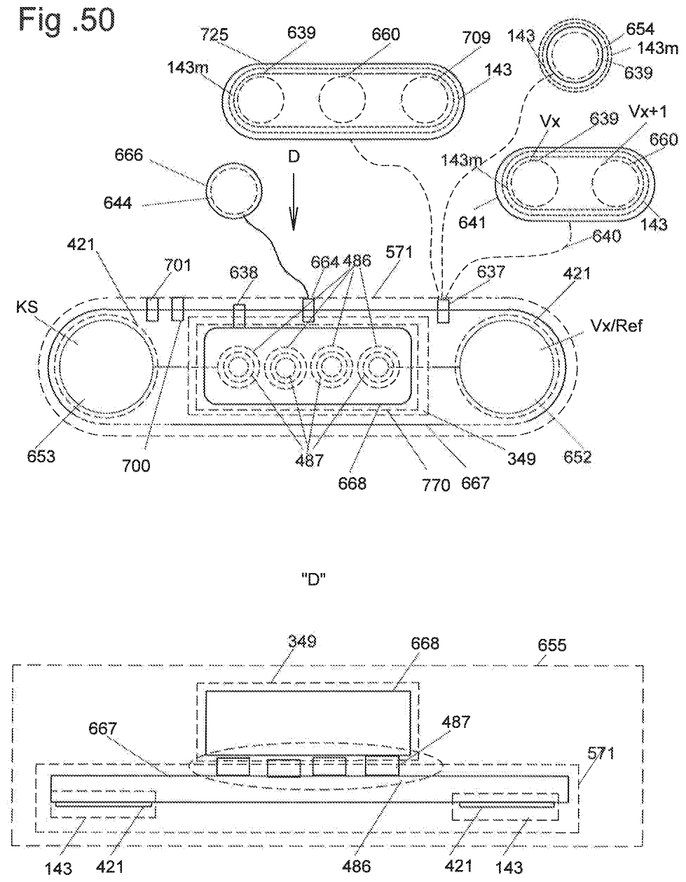

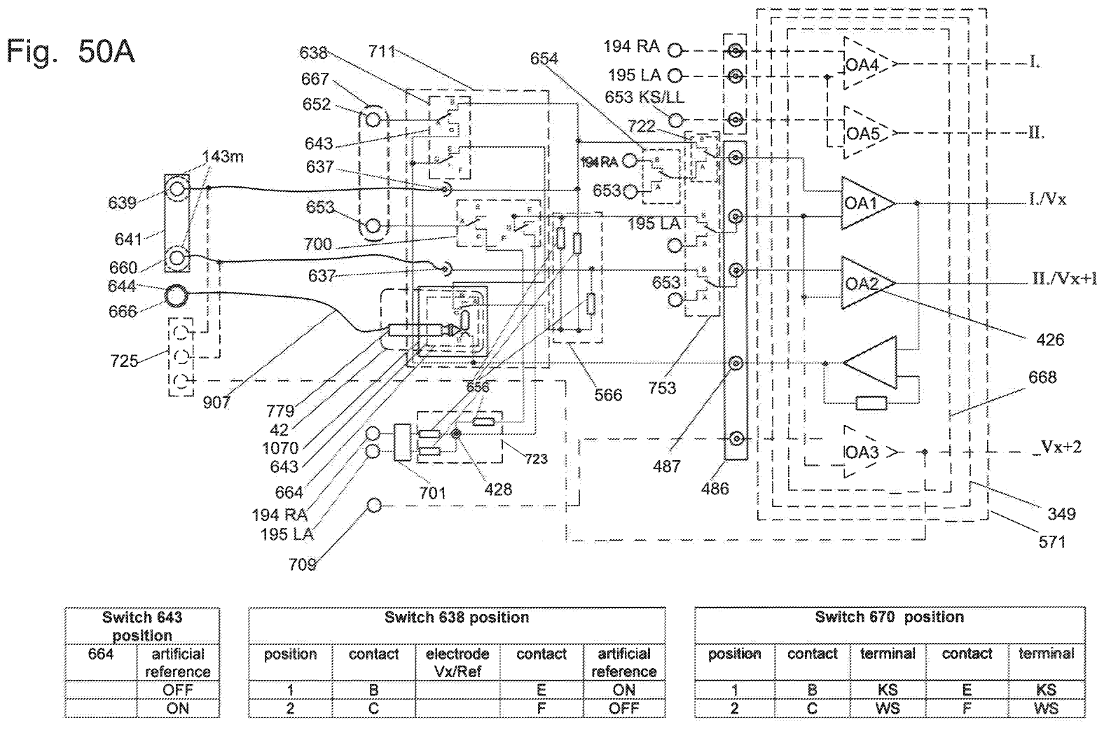

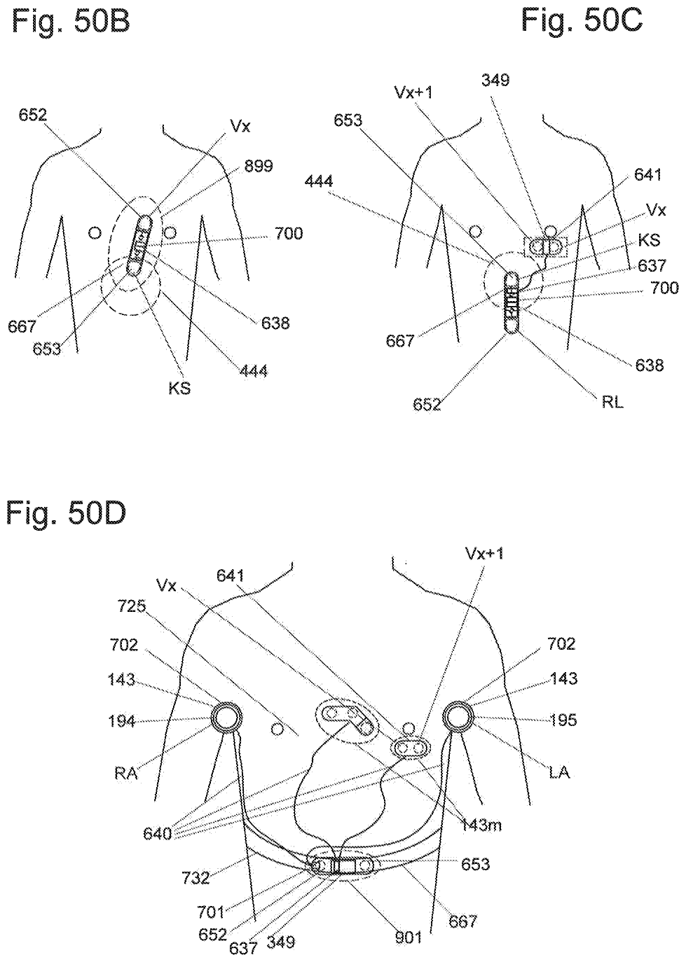

FIG. 25 shows a modification of the arrangement of the universal monitor 349 and the sensor unit 571, which is preferably formed by a base 420 or a chest belt 749, see FIG. 24, to serve for easy monitoring. The base 420 is adapted to be connected to the elastic portion 82 of the belt by means of a retaining element 563, which is preferably formed by a shaped protrusion 562 on the wrapping belt 74 and by a shaped opening 561 on the base. The base 420 of the universal monitor is provided at its edges with shaped holes 561 into which holes snap shaped protrusions 562, which terminate the wrapping belt 74. Thereby, an integral belt wrapping the body and pressing the electrodes 421 of the base against the subject's chest comes to existence. The base is adapted to house an additional battery 120 connected to the monitor 349, see FIG. 26, which battery significantly extends the time of the uninterrupted monitoring, especially in case of necessity of continuous transmission of the ECG curve. Preferably, the monitor 349 is provided with an internal pseudo-reference 566, see FIG. 50A, which can be preferably switched off by inserting of a connector pin 779 into the reference connector 643, whereby reference terminal 809 connects by cord 808 the external reference for better cardiac signal stabilization and it allows use of a less distortive filter. To save costs, the monitored person observes arrhythmia and cardiac activity traceable from the pulse on the monitor for monitoring of the heart rate, which monitor is cheaper, and in the event of deviations from the normal function, they are confirmed by measuring of the ECG at a medical center.

FIG. 26 shows a solution for the safety connection of the auxiliary battery 120 to the monitor 349. The “auxiliary battery connector 564” is designed so that when the monitor 349 is connected to the base 420 by connecting elements 486, it simultaneously connects to the auxiliary battery 120, and the monitor 349 cannot be connected to any other power source, and only then this ensures that, for example it cannot happen that due to unprofessional handling with cables, for example of some charger. Undesired application of dangerous voltage to non-isolated metallic parts, for example to contacts of freely accessible connector, or plugging of the connector with dangerous voltage into a connector allocated for the auxiliary battery 120 that is application of dangerous voltage to an area in the vicinity of the heart, resulting into danger for life in case, the monitor is connected by the connecting elements 486 to the base 42Q. An example of the shape of such case for the additional battery and of the location of such connector are shown in this figure.

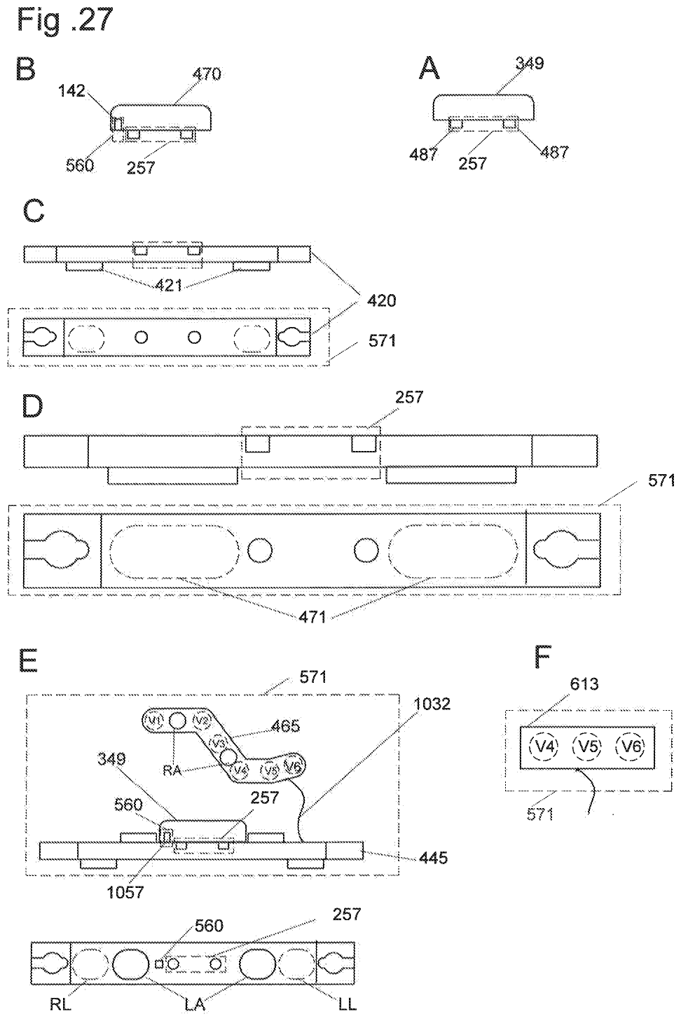

FIG. 27 shows a set of interchangeable pocket ECG units, which set is adapted for universal use of monitors 349 and sensing units 571, provided with the ability to connect monitors 349 by means of the connecting elements 257 to various sensing units in the set that cooperate with the monitors, and to connect them to the cooperating units 121 in the set using software sets. Preferably, the connecting elements 257 are formed by connecting means 580 and connecting elements 486.

Exemplary embodiments of the monitor 349 are: according to Figure A, a single-channel one with supplying of signals and mechanical fastening by means of connecting elements 486, preferably snap fasteners 487; according to Figure B, a three-channel to eight-channel one with applying of further signals by means of a connecting element 560, preferably a connector for the monitor 142, with which the base of the monitor is provided, or the signals are supplied from the electrodes by a cable.

Examples of embodiments of the sensing units 571 are: according to Figure C, a base 420 with two electrodes 421 at a distance suitable for sensing by means of a Kranz's terminal.

Figure D shows a base with two electrodes, “the electrodes 471 grouped”, for sensing of a single-lead ECG using a Kranz's terminal.

Figure E shows a sensing unit 571 formed by a chest electrode board 465, which board is connected by a cable to a base 445 of a monitor for multi-lead ECG sensing. A monitor 349 is placed on the base using connection elements 486 and they are connected by means of a connecting element 560 to the base connector 1057 to supply all signals from the board 465 via a cable 1032.

Figure F shows an example of a shortened board for sensing of chest leads. In this example, it is intended to be used for sensing of three chest leads V4, V5 and V6.

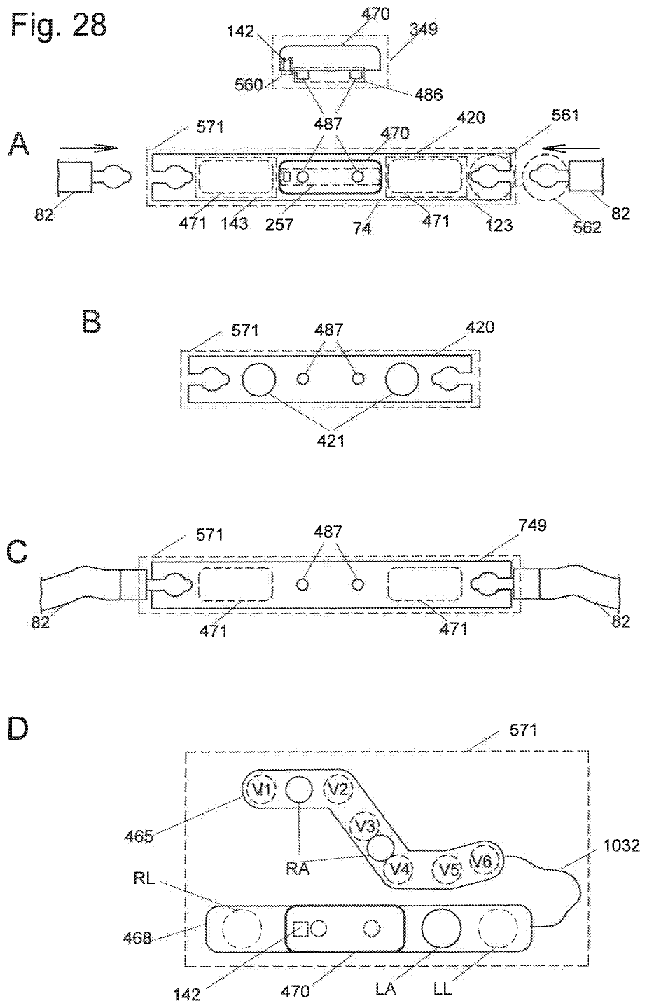

FIG. 28 shows another example of a set of monitoring devices that shows coordinated use of various monitors 349 and sensing units 571 as required. Preferably, this kit uses a monitor 349, preferably an eight-channel of cardiac signals displaying monitor 470, preferably provided with connecting elements 486, which elements allow connection of only a certain number of channels, as required, to different sensing units 571 as required. Preferably, only the required number from the total number of channels is used and the remaining ones are not used. This exhibits the advantage of using only one monitor for more sensing units, for different numbers of channels, instead of using a specific monitor with the appropriate number of channels for each sensing unit. In this example, the connecting elements 486 to the sensor unit 571 are preferably formed by snap fasteners 487, which simultaneously connect this monitor 470 mechanically and electrically by one channel, and further by a connecting element 560, preferably formed by a connector 142 for electrical connection of electrodes for the remaining 7 channels. This allows to connect the monitor in Example A to the sensing unit 571, preferably formed by base 420 for the Kranz's terminal, preferably made of some flexible material to cope the chest shape, where preferably only the snap fasteners 487 are used to connect two ECG electrodes 143 on the sensing unit 571 with the snap fasteners for connecting of one channel of the monitor 349. In this example, the electrodes are designed for the Kranz's terminal and they are formed by collecting electrodes 471 that are grouped, which electrodes allow by attaching them to chest to sense the grouped cardiac signals for a short period of time, approximately for curve I, V3 to V6, on which curve also elevations of the ST segment are visible. The base can be secured by shaped holes 561 to shaped protrusions 562 on the wrapping belt 74 for fastening it to the chest for long-term sensing. The advantage of base with the grouped electrodes are small dimensions and simple and fast application even by a layman, even in the case a wrapping belt 74 is added to fasten to the chest for long-term sensing. It is suitable for the preliminary detection of the possible occurrence of elevated ST segments and of other deviations from the ECG normal for prevention of AIM or of the possible occurrence of irregular pulses for the prevention or control of arrhythmia.

In the kit shown in Example B, there is another sensing unit 571, which allows sensing by means of a Kranz's terminal, in this time by electrodes for individual chest leads V1 to V6, with sensing by successive attaching them to the chest, preferably formed by the base 420.

The set according to Example C includes also a chest belt 749. Said sensing units do not use the connector 142

If necessary, especially when elevated ST segments are detected, or irregular pulses occur, the monitor is moved to the sensing unit 571, which is preferably formed by a multi-channel sensing base 468, for sensing, preferably, of eight channels, as it is shown in Example D for short-term applications by attaching it to the body. With which it is possible to sense all 12 leads at once, namely the lead I (from RA, LA), II (from RA, LL), V1 to V6 against the Wilson's terminal, and 4 leads can be calculated, as it is shown in FIG. 12.

To the base 468 for sensing of 8 channels for sensing of LA, LL, RL is by a cable 1032 connected the board 465 of chest electrodes. A complementary counter-piece of connector 142 is located on the base 468, which brings to the monitor 470 connection for 10 ECG electrodes 143 located on the board 465.

Relocation of monitors between sensor units 571 is enabled by connection elements 257, preferably formed by connection elements 486, preferably snap fasteners 487, and/or connection elements 560, preferably by connector 142 and software for the kit allowing to communicate between monitors 349 and cooperating units, which are included in the kit. In the described Examples A through D of the kit, only several monitors 349 and sensing units 571 are shown, but multiple monitors or sets may be used as needed.

In the case of connecting the electrodes 143 to the single-channel monitor, preferably only snap fasteners 487 are used on the base 420 with the grouped electrodes. On the 8-channel sensing base, an 8-channel connector 142 is preferably used to connect the electrodes 143, and in this example, the snap fasteners are used only for mechanical mounting of the monitor. For the short-term sensing, the boards and bases are placed against the chest and they are held by fingers.

For the long-term sensing, preferably, the sensing units 571 are connected to a wrapping belt 74 or a chest belt 749 is utilized.

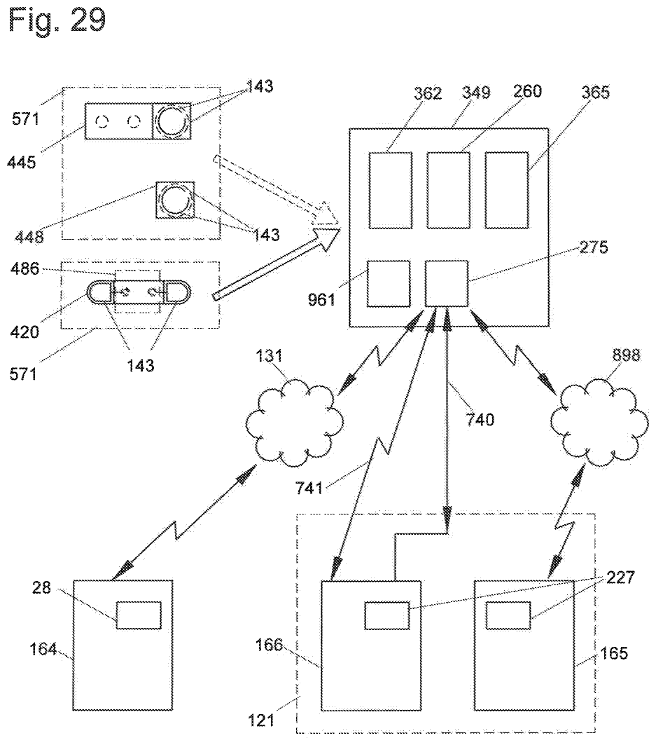

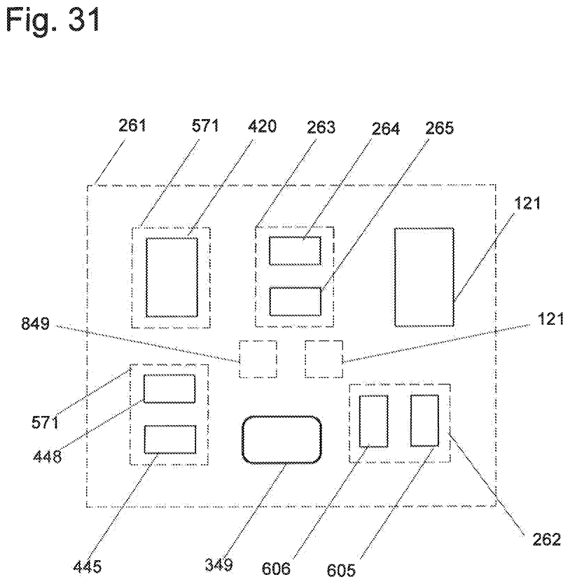

FIG. 29 shows a block diagram used to explain the principle of a pocket ECG, in particular for checking whether there is an infarction state, which pocket ECG is adapted to be carried by a monitored person in a pocket ECG carrying means 417, preferably in the cardio first aid kit 261 shown in FIG. 31 or in a pocket or a small bag or a bag, and, if necessary, verifying of the infarction diagnosis, it is possible to use the pocket ECG, which pocket ECG is designed to monitor at least one of the chest leads V1 to V6, to monitor especially the symptoms of AIM immediately.

For deployment speed, the ECG electrodes 143, located on the pocket ECG sensor unit 571, are simply attached to the body of the monitored subject, and they are held by hands. This saves valuable time, if compared to the more permanent fastening, such as with belts, by gluing or by clipped terminals. Alternatively, the sensing unit 571 is secured by a belt for continuous monitoring.

To make the diagnosis rapid, preferably, for a preliminary AIM assessment, the simplest method of a single-lead ECG monitoring of the pseudo-chest leads, which are sensed from the chest by ECG electrodes 143 using the Kranz's terminal base 420 is used.

The monitor 349 is placed on the base 420, and, preferably, it is fastened to it by connecting elements 486 or it is connected to it by wires, and it is placed externally, for example behind a belt. The Kranz's terminal exhibits the advantage consisting in the simplicity of sensing the chest leads with only two electrodes, and thus also in the speed of diagnosis, what compensates for the inaccuracy of this sensing.

In case of suspicion or indication of an AIM diagnosis, to clarify the diagnosis, the Wilson's terminal is preferably used for sensing of the chest leads, preferably by means of a sensor unit 571, preferably formed by “the base 445 of a monitor for multi-lead sensing”.

The switching field 711 allows sequential sensing of up to 12-lead ECG also by using a less-channel monitor, e.g. by a single-channel monitor 349. For a change from sensing with a Kranz's terminal to sensing with a Wilson's terminal, preferably, the monitor is moved from the base 420 for the Kranz's terminal to the base for sensing with the Wilson's terminal, which base preferably comprises a small board 448 with two electrodes and a base 445 as it is described in FIGS. 17 and 19.

Preferably, various optional sensing units 571, monitors 349, chargers, replaceable batteries to facilitate field monitoring, are located in the pocket ECG carrying means 417.

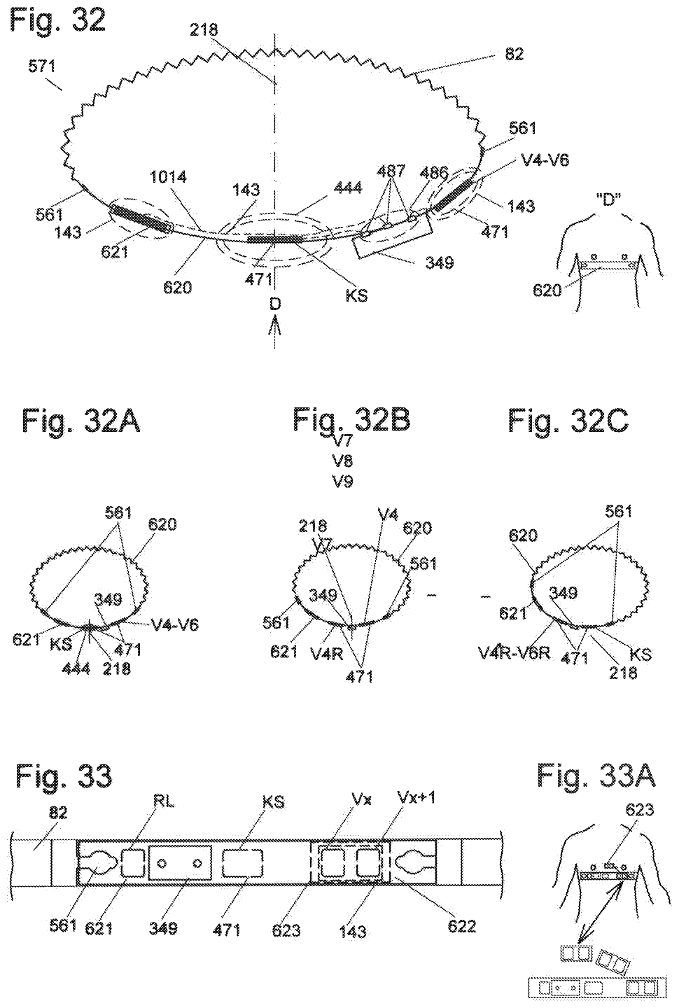

Preferably, the monitors 349 and the sensing units 571 are interchangeable to allow the possibility to combine them, and they form a set. The units 571 and monitors in the carrying means 417 may be initially used in a lower number and as the less expensive ones to reduce the costs and they may be replenished over time as needed. Preferably, the carrying means 417 is the cardio first aid kit 261, which is described in FIG. 32.

From the sensing units 571, the signal is transmitted to the monitor 349, which monitor includes a front end 362, filters 260, control units 365, a memory 961, and a communication unit 275. The signal from the monitor 349 is transmitted via a wired link 740 and/or wirelessly to the local cooperating units 166 and/or to nearby cooperating units 164 and/or to remote cooperating units 165.

The wireless transmission to the nearby cooperating units 164 is preferably realized via Bluetooth or ZigBee or via a Wi-Fi network 490. The wireless transmission to the remote cooperating units 165 is preferably carried out via networks 898 of mobile operators. The actually sensed or stored ECG waveforms are displayed on the displays 28, 227 of the cooperating units. The cooperating units 121, both the local ones 166 or the nearby ones 164 or the remote ones 165, may be formed by pocket units 230, preferably by those formed by a mobile phone, and by desktop units 231, which are preferably formed by a server 806 and/or a PC 962, as they are shown in FIG. 11.

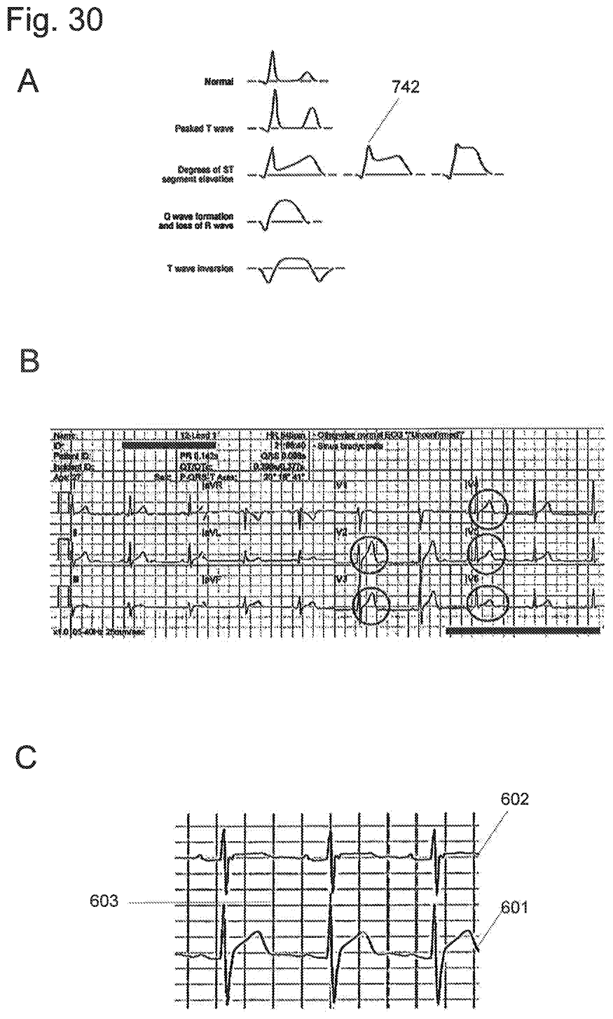

FIG. 30 shows the use of a pocket ECG for the diagnosis of acute myocardial infarction (AMI) by a monitored subject. Such layman is instructed by medical staff or provided with instructions to assess a heart attack. For the diagnosis of AIM, it is important to detect AIM changes 742 on the ECG, i.e., the ST segment elevation, the enlarged T waves, the altered Q wave shape together with the R wave loss, or the T wave inversion, as they are shown in Example A. Example B shows the ST segment elevation on the ECG curve, which is best seen from the chest leads V2 to V6, as it is highlighted by circling. Therefore, for the assessment of AMI, mainly the chest leads are important, not only the V1 to V6, but also the V3R to V6R and the V7 to V9, without which an AMI cannot be ruled out. The simplest way to evaluate is to compare the actual ECG 601 with a master ECG 602 taken by the monitored person at a time when the AIM did not occur, as it is shown in Example C.

For displaying in the field, the pocket ECG uses pocket cooperating units 121, preferably an evaluation and display unit 764, preferably formed by a mobile phone, which mobile phone has a relatively small display when compared to the one of the desktop co-operating units, preferably of a PC. It is provided with a relatively small display, where it is difficult to display a full 12-lead ECG for comparison. Therefore, only the individual leads of the “actual ECG 601” and the “master ECG 602” are preferably displayed to detect changes in the AIM 742, preferably, they are displayed below each other for comparison, as it is shown in Example C. Alternatively, the actual and the maser ECG can be displayed on two displays 227, or on a split display of one local cooperating unit, or on two units side by side, which are adapted to display the actual and the new status. The “elevation of ST segment 603” is visible in this example on the curve of the “actual ECG 601”. If a sample ECG of the monitored person is not available, a sample ECG of a healthy individual is displayed, which ECG is preferably available in the memory of the cooperating unit 121.

Another method is to detect the possible changes in the AIM 742, such as the “ST segment elevation 603”, or the ST segment depression, which would indicate such AIM visually from the actual ECG without any master ECG. To detect elevation and other deviations from the normal ECG, automatic pocket ECG diagnostics may be used, preferably performed by the monitor control unit 365 or the unit 764, which, in the event of ST segment elevation or other AIM 742 changes from the normal ECG, will preferably trigger a warning signal and display ECGs with a description of the diagnosis, which are forwarded to cooperating units 165, preferably to the remote ones, via a server to medical personnel automatically, or only when the monitored person does not reset the warning signal.

Alternatively, the ST segment is evaluated in the cooperating units 121 automatically or manually, preferably at the request of the monitored person, by medical personnel, and the results are forwarded back to the monitored person, preferably to the unit 764.

FIG. 31 shows a CARDIO first aid kit 261 (CARDIO FIRST AID), which is preferably carried by the monitored person and which is used in case of preventive monitoring or in case of symptoms of AIM or arrhythmia for control diagnosis.

Its content is optional and essential are the diagnostic means for diagnosing any possible infarction or arrhythmia and the first aid drugs in case of confirmation of the diagnosis of infarction or arrhythmia.

Of the diagnostic tools, it is necessary to have at least the simplest pocket ECG, which will detect the symptoms of AIM, and preferably arrhythmias. That is, such ECG that monitors at least the pseudo-chest leads, what preferably is a sensing unit 571 for a Kranz's terminal, which unit is preferably formed by a small board 448 complemented by a monitor for at least one channel.

In the case of an AIM diagnosis, the ECG is sent for review by medical staff by means of the cooperating units 121 and the base 445. An advantage is to equip the first aid kit with a sensing unit using the Wilson's terminals, which unit monitors the chest leads more accurately, and with a cooperating unit.

A sensor unit 571, preferably formed by a sensor unit 420 for the Kranz's terminal, is preferably used for the pre-monitoring, and in case of occurrence of the AIM symptoms, the monitor is moved into a Wilson's terminal comprising unit 571 having ability to obtain a 12-lead ECG.

An inexpensive single-channel monitor 849 is sufficient to equip the first aid kit to sequentially scan individual ECG leads. With a two-channel or a three-channel monitor 369, multiple leads can be monitored at once. The most expensive 8-channel monitor 349 allows to sense 8 leads at once and simultaneously to calculate 4 other leads to display 12 channels live simultaneously.

When diagnosing the AIM, it is necessary to use a “chemical test pocket kit 262” preferably containing “Troponin I 606” and/or “Troponin I/Myoglobin 605” to confirm the ECG findings.

Preferably, these drugs are included in the first aid kit, and the monitored person can performed the chemical tests by himself/herself, and so to speed up the confirmation of the diagnosis in comparison to performing the tests not sooner than upon arrival at the medical facility. Sending of an ECG and possibly of the results of chemical tests to a cardiology facility, such as to an ambulance or hospital, will facilitate the diagnosis and decision about transport of the monitored person to a hospital in accordance with the level of threat.

Preferably, the first aid kit also comprises medicaments 263, which the monitored person will use in the event the AIM is confirmed according to instructions of the contacted medical staff.

Preferably, the relevant medicaments are blood thinners, e.g. Aspirin 264, painkillers 265. For sending of an ECG to medical facility and for communication, preferably, the first aid kit comprises a cooperating unit, preferably a mobile phone, which is independent of e.g. the mobile phone for common communication, which independent mobile phone provides for sending of ECG and for communication especially in case when the commonly used mobile phone does not function as a backup.

Preferably, the first aid kit comprises a device for checking of the physical and mental fitness of the monitored person, which person has to show physical activity or to reset the “count down” timer, otherwise the device will trigger an alarm in the medical facility and will report the GPS coordinates or other data about location of the monitored person.

In the beginning, the first aid kit can be very simple and more equipped more gradually.

FIG. 32 shows a sensing unit 571, adapted both for attachment and carrying, used for ECG sensing with a single-channel monitor 349 using an RL reference, preferably formed by a “RL base 620”, preferably in the shape of a chest belt, provided with connection elements 486 for electrical and mechanical connection of the monitor 349. The monitor 349 is provided with one output, which is connected to an electrode 143, preferably formed by an “RL electrode 621” for injecting of an RL reference signal.

The figure shows a variant where this signal is fed to the electrode 621 by a cable 1014 running along the base 620, but it may also be fed through the base 620, preferably to a connecting element 486, preferably formed by a snap fastener 487, which mediates contact of the electrode 621 for RL with the monitor 349. In this case, elongated large-area collection electrodes 471 of 10 to 150 mm in length are used to sense grouped cardiac signals corresponding to multiple ECG leads, wherein, one of the electrodes 471 senses potential from the Kranz's terminal area 444 and the other one that in the lead V sensing area, wherein it senses the grouped signal of V4 to V6, in particular to display the elevation of the ST segment.

The base 620 is attached to the body and held for the short-term sensing, or by means of shaped holes 561, an elastic portion 82 of the belt is attached on both sides, which portion encircles the chest and adheres the base 620 to the chest for the long-term sensing.

In the position of the base 620 shown in FIG. 32, which corresponds to FIG. 32A, the leads V4 to V6 are sensed against the Kranz's terminal (KS) using the RL as reference.

FIG. 32A shows the base 620 with the grouped electrodes 471 located on the chest as shown in FIG. 32. The electrode 471 located to the left off the chest center 218 senses the grouped signals V4 through V6 against the electrode 471 located in the chest center 218 in the Kranz's terminal area 444 to form a Kranz's terminal (KS) showing the elevation of the ST segment.

Electrode 621 is used for reference.

FIG. 32B shows the base 620, shifted to the right on the chest, with the electrodes 471 located symmetrically to the left and right off the center of the chest 218 to sense the grouped signals from the chest spots recommended for sensing of leads V3, V4 against signals from sensing spots V3R, V4R. This allows to display elevation of the ST segment grouped for all said leads.

FIG. 32C shows the base 620 shifted on the chest even further to the right, with an electrode 471 located in the center 218 of the chest in the Kranz's terminal area 444 and a second electrode 471 in the area recommended for sensing of leads V4R to V6R, wherein, it allows sensing of grouped signals of leads V4R to V6R against Kranz's terminal, especially for displaying of the elevation of the ST segment. The electrode 621 is used for reference.

FIG. 33 shows a “base 622 for sensing of RL and 2×V” allocated for sensing of two leads V by a two-channel monitor 349 against the Kranz's terminal and under using of the reference RL, by means of the electrode 621. A “sub-board 623 for 2×V”, containing two electrodes 143 for sensing two leads V, Vx and Vx+1, can be mounted on the base 622. When the sub-board 623 is attached to the base 622, it is in a position intended to sense the leads V4 and V5. Preferably, the sub-board 623 is removable from the base 622 and can be moved to sense always a pair of leads V, i.e. of the leads V1 and V2, V3 and V4, V5 and V6, and similarly to sense right-chest leads, with the suffix R in the designation, and the back leads, so as it is shown in FIG. 33A. The sub-board 623 is connected to the base 622 by a cable. Therefore, the two-channel monitor 459 always transmits two V leads at the same time.

FIG. 33A shows the attaching of a “sub-board 623 for 2×V” for sequential sensing of leads V1 and V2, V3 and V4, V5 and V6. Other leads V3R and V4R, V5R and V6R, V7 and V8, V8 and V9 are sensed similarly.

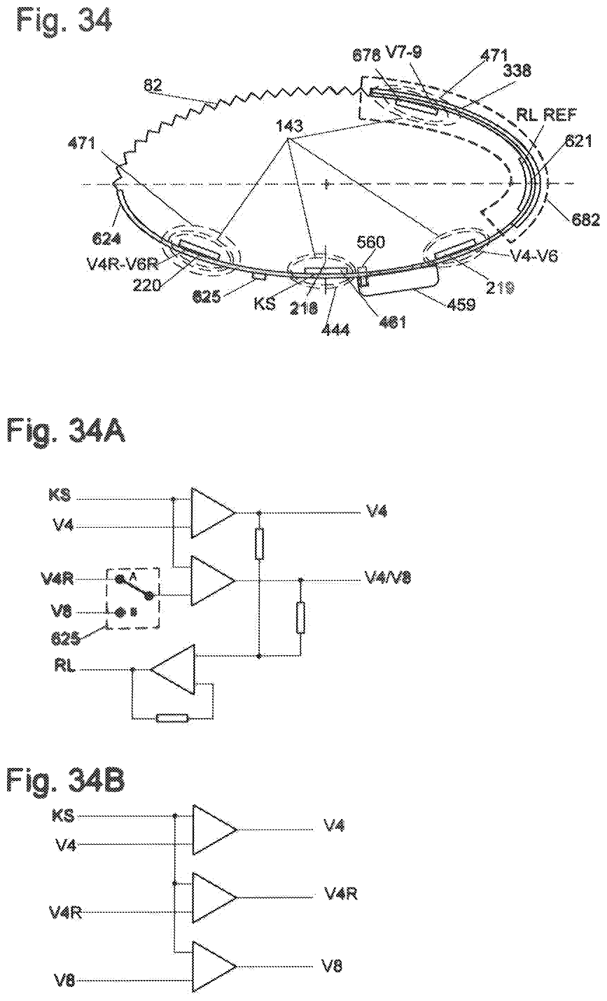

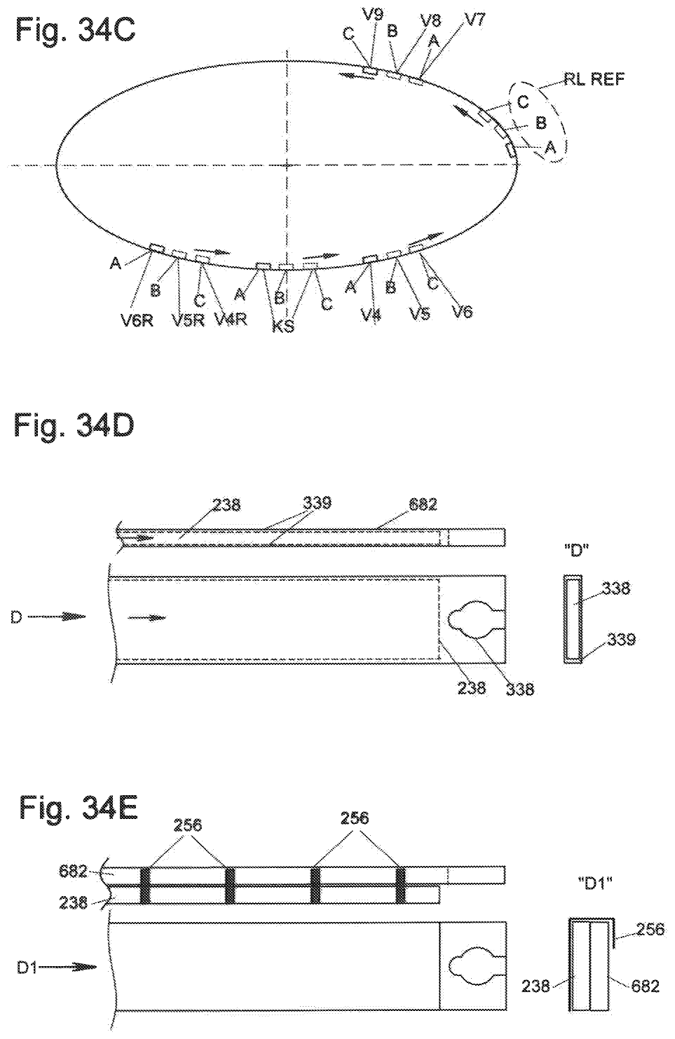

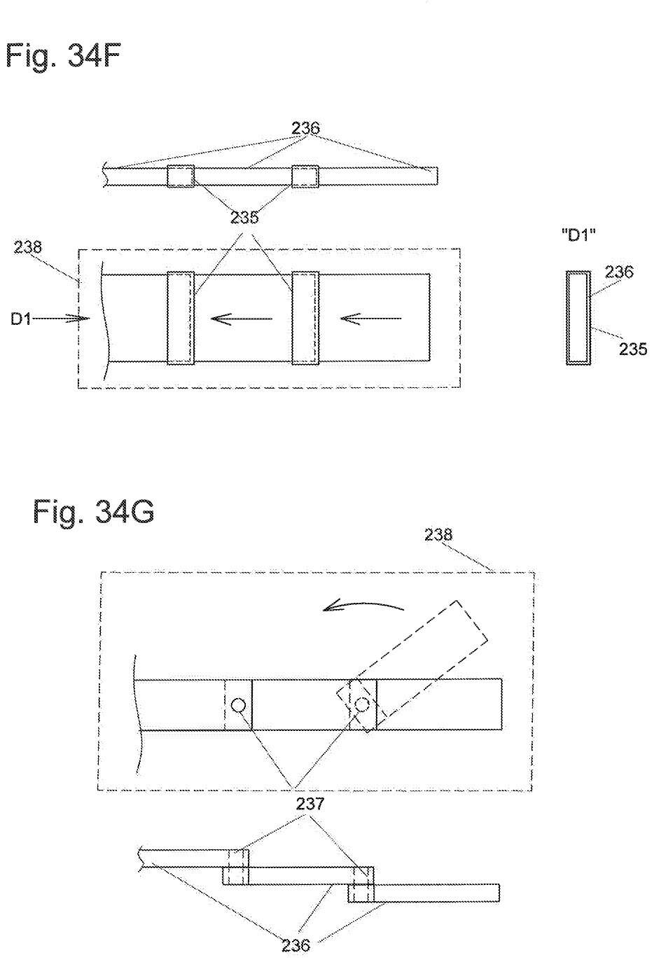

FIG. 34 shows an “extended base 624”, which is provided with electrodes 143, preferably with the grouped electrodes 471, of which one, preferably the one formed by the electrode 461 for sensing of potential of the Kranz's terminal, is located in the chest center 218 for sensing from the area 444 of the Kranz's terminal. Another electrode 143 is preferably formed by an electrode 219 for the chest leads V4 to V6, another by an electrode 220 for the right side chest leads V4R to V6R, another electrode 621 is for the RL reference, and another electrode 678 is for the back leads V7-V9. Completion of the commonly sensed leads V4 to V6 with leads V4R to V6R and V7 to V9 is important for capturing of elevation of the ST segment.

Preferably, the extended base 624 is made of an elastic band-shaped material, which is flexible around the longer side of the cross section and sufficiently rigid to twist to allow to insert its electrode 678 for sensing of the back leads V7 to V9 with the left hand under a shirt, T-shirt or clothes.

After insertion to the chest, the extended base 624, fitted with the monitor 459, may be held attached on the chest by hand of the monitored person. Alternatively, only the extended portion 682 of the base is made of the elastic material and the remaining part is predominantly of fabric and is off the electrodes 143 and the connecting element 560.

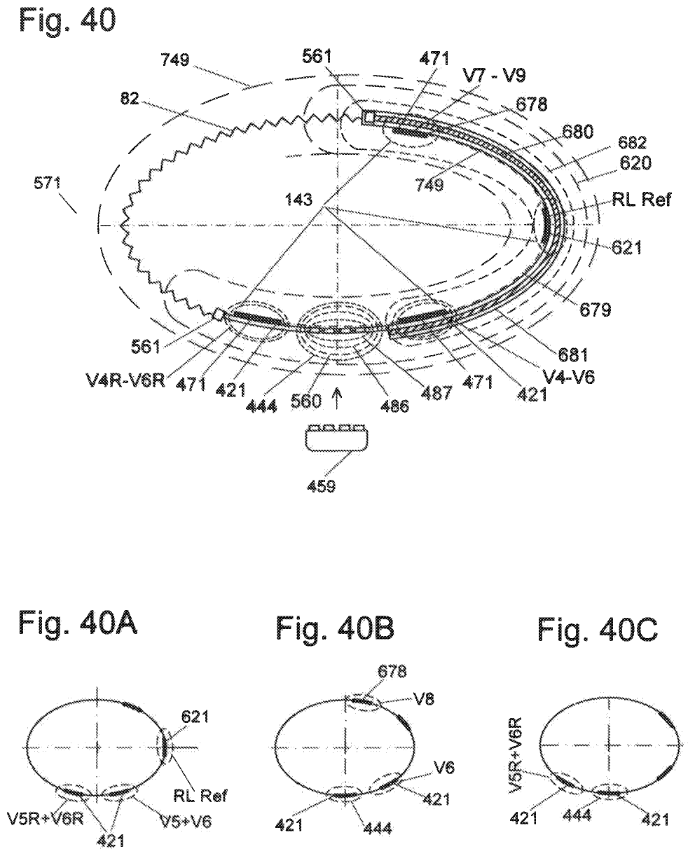

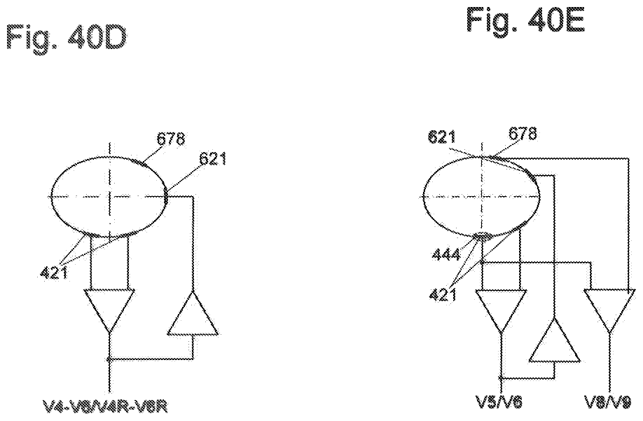

Preferably the extended part of the base 624 is made of a fabric material, it is strengthened by a brace of the extending part 682, which is preferably detachable, so as it is shown in FIG. 40, and thus preferably the entire base is made of a fabric material. To place it into a pocket, the fabric extended base 624 is for example rolled up and the elastic extending part 682 is preferably composed of smaller parts that are extended preferably by insertion into each other or by folding it up so as a folding rule to fit in the pocket when folded. When sensing the lead V8, it is necessary for the monitored person to provide pressure to the electrode 678 for back leads V7 to V9, preferably by resting of the person against a chair backrest or by lying down. Alternatively, the elastic portion 82 of the belt is attached, which form a complete chest belt with the extended base 624 to exert pressure to all electrodes on the subject's chest and back.

Preferably, the extended base 624 is equipped with a dual channel monitor 459, the block diagram of which is shown in FIG. 34A, with electrode connections 219 and 220, and in case of the desired connection of the electrode 6 for the back leads V8, this electrode is connected to it by means of a switch 625 instead of the electrode 220 for sensing of V4, or a three-channel monitor is used, the block diagram of which is shown in FIG. 34B.

Alternatively, instead of the switch 625, the extended base 624 is provided with the electrode 220 for the leads for sensing of V4R on the right side of the chest, which electrode is displaceable to the position V8. The electrodes 461 for sensing of the Kranz's terminal (KS), the electrode 219 for sensing of the V4, and the electrode 220 for sensing, of the V4R, and the electrode 678 for sensing of the V8 are preferably formed as the grouped electrodes 471 sensing the grouped leads V4 to V6, V4R to V6R, V7 to V9, or as electrodes intended for sensing of only one lead, preferably of V4, V4R and V8, and by a double horizontal rotation of the base 624 in the direction of the arrows to the recommended places for sensing of individual chest leads, it is possible to sense all chest and back leads sequentially, as it is shown in FIG. 34C.

FIG. 34A shows a block diagram of the input circuitry of the monitor 349 with the switch 625. The dual-channel monitor senses the chest lead V4 against the Kranz's terminal and the other lead, either the V4R or V8 lead, according to the position of the switch 625, so as it is shown in this figure. For an alternative use to sense 3 leads V4, V4R, V8 at the same time, the switch 625 is omitted and a three-channel monitor is used. Preferably, the reference signal RL is applied to the electrode 621. In the case of monitoring with a pseudo-internal reference, the external reference and the electrode 621 are not used.

FIG. 34B shows a block diagram of a three-channel monitor 459 for simultaneous sensing of leads V4, V4R, and V8 against the Kranz's terminal. The reference is not drawn.

FIG. 34C shows an alternative solution where the electrodes 471, 621 and 678 of FIG. 34 are not formed as the large-area electrodes, but as electrodes that are shortened to the size for individual leads, in the initial position, they are located at the points marked “A”. Then the sensing can take place in three sequences by rotating the extended base 624 on the chest, for example from right to left, where in the initial position of the electrodes, which position is marked A, the leads V6R, V4 and V7 are measured, then, after the first rotation into the position “B”, the leads V5R, V5 and V8 are measured, and after the second rotation into the position “C”, the leads V4R, V6 and V9 are measured, and this always against the Kranz's terminal, preferably with using of the electrode for reference RL REF, which is located on the left side, by the electrode 621.

FIG. 34D shows “the extending portion 682 of the base”, preferably of the plastic or fabric base 624, with the electrode 678 for a back leads, which electrode is insertable under the garment of the monitored person, which electrode is attached firmly to the extended base 624 and provides sufficient rigidity and length for insertion. It can be rolled up for carrying in a pocket. To facilitate insertion of the extending portion 682 under the garment up to the back leads, a brace 238 is inserted into the “extension portion 682 of the base”, which has a doubled wall 339, to provide the desired mechanical properties of the base 624.

FIG. 34E shows an alternative solution in which the extension portion 682 is of the same material as the rest of the base 624, but a brace 238 may be attached thereto. The brace 238 is fixed to the base preferably by clips 256.

Preferably, the brace 238 is of one piece, preferably flexible enough to be rollable.

FIG. 34F shows a brace 238, which is formed of brace segments 236, in order to minimize its dimensions, which, when folded into a line, always in the direction of arrows, fit into each other, preferably by means of a shoulder 235, as it is indicated in this figure.

FIG. 34G shows a brace 238 which is formed by brace segments 236 and which is collapsible by means of joints 237 on the principle of a folding rule. Thus, in the folded state, it has the length of one brace segment 236.

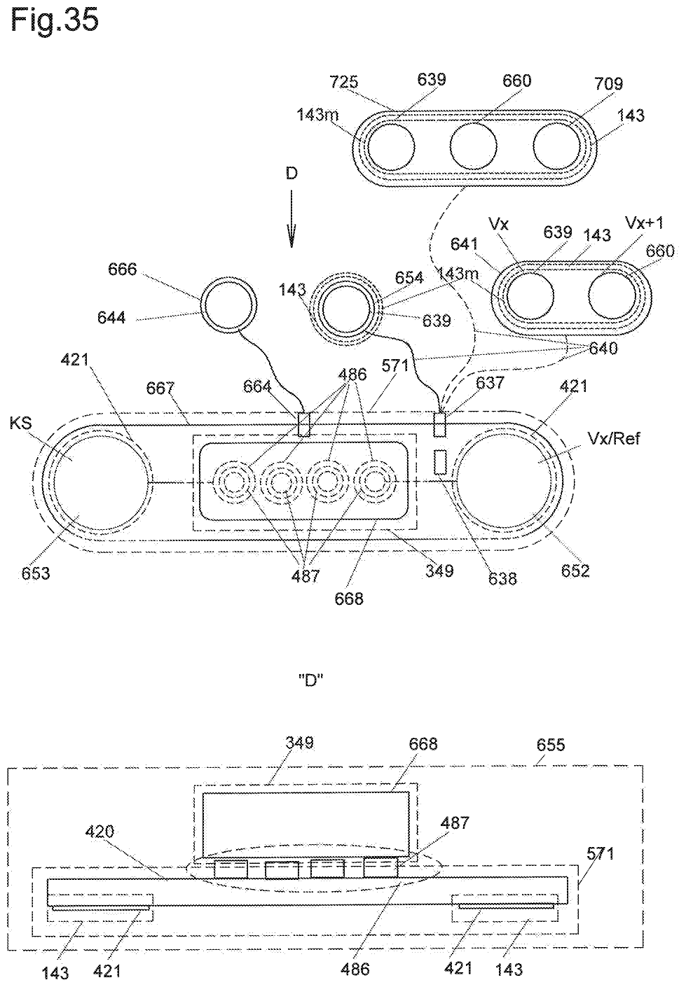

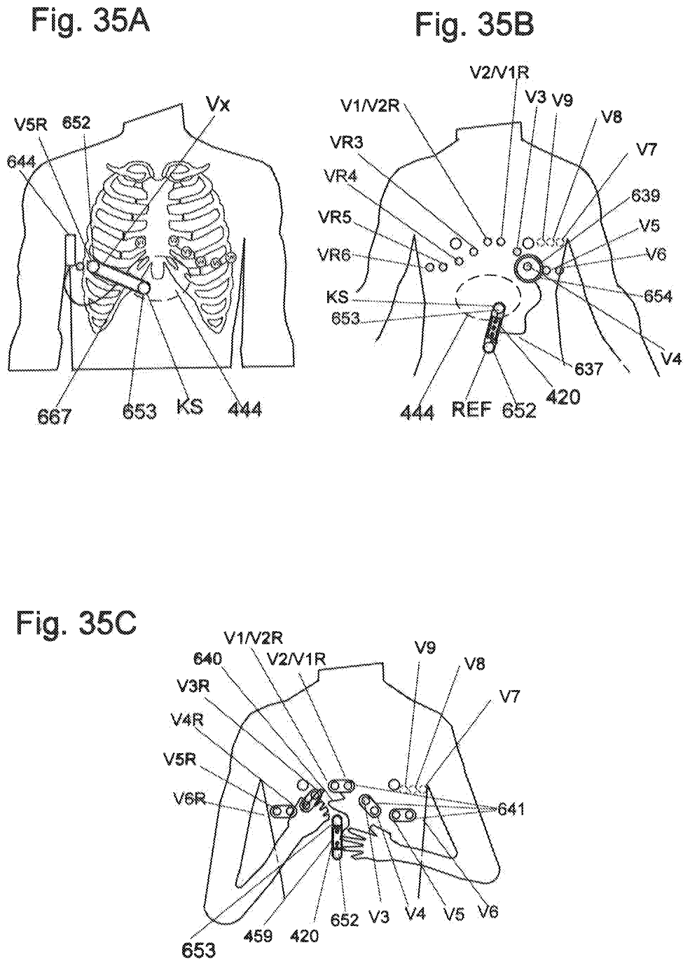

FIG. 35 shows a sensor module 655 consisting of a monitor 349 and a sensor unit 571 for sensing of the chest leads V1 to V6, V1R to V6R, and V7 to V9 by means of a Kranz's terminal, preferably using a reference electrode 644. Further, this Figure shows a “base 667 with a reference” of the universal monitor 349, preferably of the single-channel one, which base is provided with electrodes 421, preferably with one electrode formed by the electrode 652 for Vx/REF leads for sensing of the chest leads Vx, which is switchable by the “switch 638 Vx/REF” to the reference REF, and simultaneously disconnectable from the input of the operational amplifier 406, which is connected to the connector 637 and connected by cable 640 to the electrode 639, which electrode is connected for sensing of the Vx in case the electrode 652 is switched to the Ref.

Alternatively, the cable 640 connects the electrodes 639 for Vx and 660 for Vx+1 to the electrode board 641 for a dual channel monitor for sensing, preferably of all chest leads optionally, by relocation. Preferably, the second electrode 421 is formed by the electrode 653 for the Kranz's terminal/LL, i.e. by the electrode for sensing of the potential of the Kranz's terminal, alternatively for the signal from the left leg LL. The electrodes are formed by round or elongated large-area grouped electrodes 471 shown in FIG. 32 or by electrodes of smaller size for sensing of individual leads.

Preferably, the base 667 serves for fitting with a single-channel monitor or a dual-channel one.

The small board 654 of the electrode on cable or the small board 641 of the electrodes for a dual-channel monitor are connected by means of the connector 637 for Vx. Alternatively, the small board 725 of three chest electrodes can be connected for cooperation with a three-channel monitor.

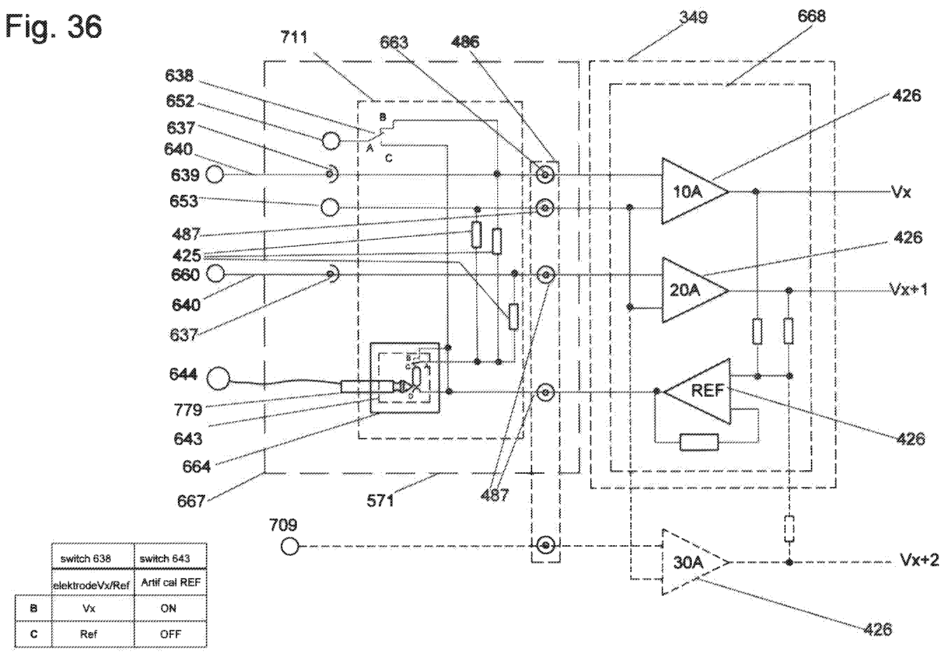

By means of the reference connector 664, a reference electrode 644 can be connected to the reference board 666 instead of an artificial reference, which is disconnected by the “reference switch 643”, preferably automatically when the pin of the reference connector 664 is inserted. Thus, the base 667 includes a “reference switch 643” and preferably a “638 Vx/REF switch” for various sensing options, as it is shown in FIGS. 35A-35D and the table in FIG. 36.

The Detail “D” shows composition of the sensing module 655 formed by the sensing unit 571 and a monitor 349 of the cardiac signals, which is attached to the sensing unit and at the same time conductively connected to it by means of connecting elements 487.

FIG. 35A shows sensing of the chest leads against the Kranz's terminal using the base 667. The electrode 653 is attached to the area 444 of the Kranz's terminal and the electrode 652 Vx/REF is attached to spots recommended for sensing of the chest leads Vx, in particular in this Figure of the right side chest lead V5R. At the same time, the reference electrode 644 on a cable is used, which electrode is preferably grasped in the right-side armpit.

FIG. 35B shows the use of the base 667 for sensing of chest leads by means of the small board 654, wherein, with for reference the abdomen area is used by attaching of the electrode 652 for the V2/REF, which electrode is switched by the switch 638 for Vx/REF to the position C ref. The switch 643 in FIG. 36 is in the position C, when the feedback resistors 425 are not used and supply of the reference signal is provided by the electrode 652, which electrode is set to the reference electrode mode, as it is shown in the block diagram in FIG. 36.

By means of the small board 654 of the electrode on a cable connected to a monitor via the connector 637 of the electrode Vx, all chest leads shown are sensed consecutively so as it is shown in FIG. 35B.

FIG. 35C shows the use of the base 420 for sensing of leads Vx, always two at a time, using a two-channel monitor 459 and a small board 641 of electrodes for the two-channel monitor.

FIG. 36 shows a block diagram of the input circuitry of the monitor 349 of FIG. 35, preferably formed by a monitor 668 with an output of the reference. The sensor units 571, preferably formed by the base 667 and the monitor 668, which is mechanically and electrically connected to the base 667 by four connecting elements 486, preferably formed by snap fasteners 487. The switch 638 Vx/REF switches the electrode 652 from the Vx to the Ref.

The reference electrode 644 is connected via the reference connector 664, which preferably automatically switches the switch 643 when the connector 779 of the pin is inserted, what automatically switches the switch 643, which disconnects the pseudo-reference. The electrode 639 for Vx on the “board 654 of the electrode on the cable” and the electrode 660 will be connected via the connector 637. This allows sensing of one chest lead by the ECG electrode 652 or by the electrode 639 on the cable 640, or of two ECG leads by the electrodes 639 and 660 on the cable 640. It is sensed either with an artificial reference, generated by resistors 425, or with a reference signal injected in the right side armpit of the monitored person by the electrode 644 or by the electrode 652, which is switched by the switch 638 to the reference mode.

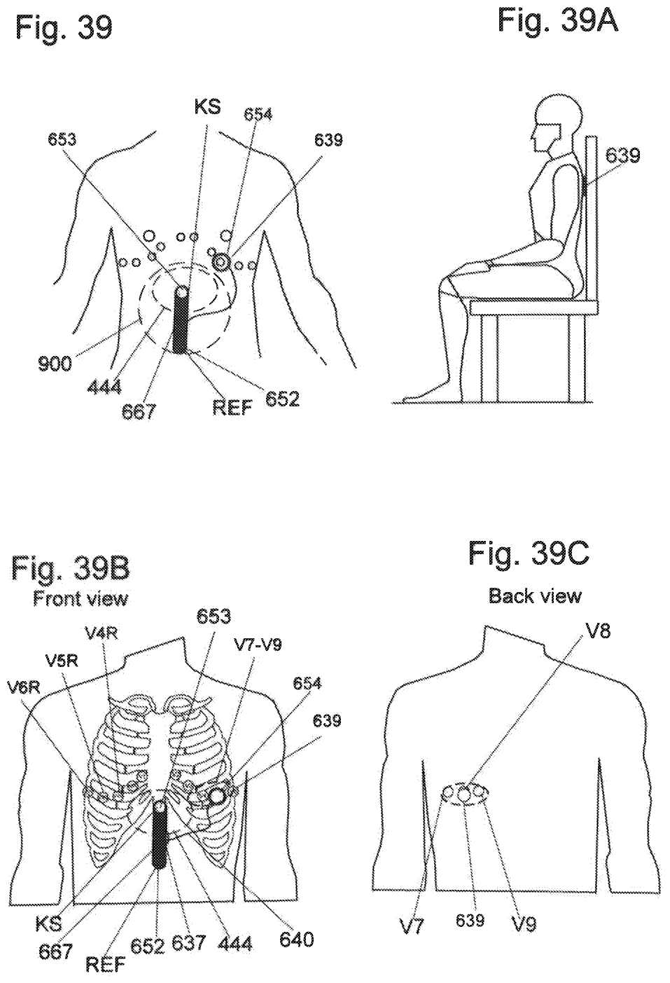

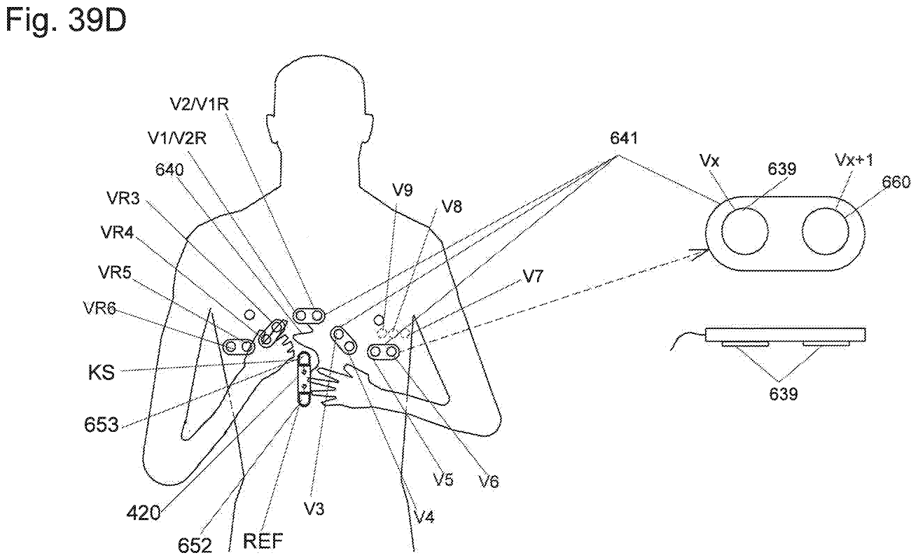

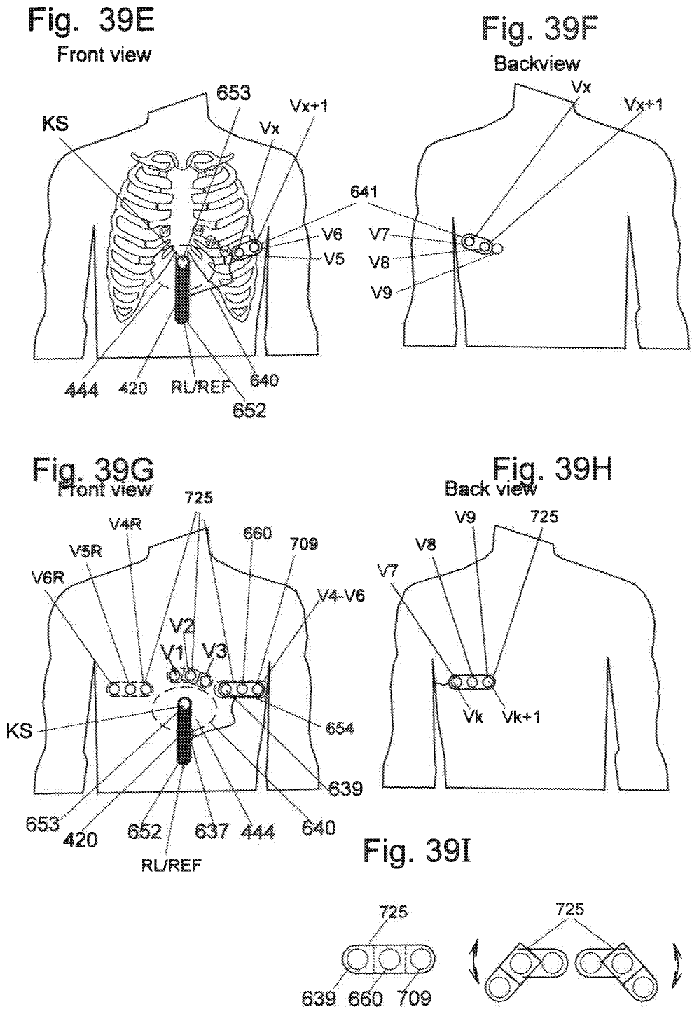

The Table shows positions of the switches 638 and 634 for sensing of the Vx or the use of an artificial reference. The dashed lines show a third operational amplifier 626 for the Vx+2 electrode 709, which is preferably added to the electrodes served by the first two operational amplifiers 626 for sensing 3 of the Vx leads at once, as it is shown in FIG. 39G.

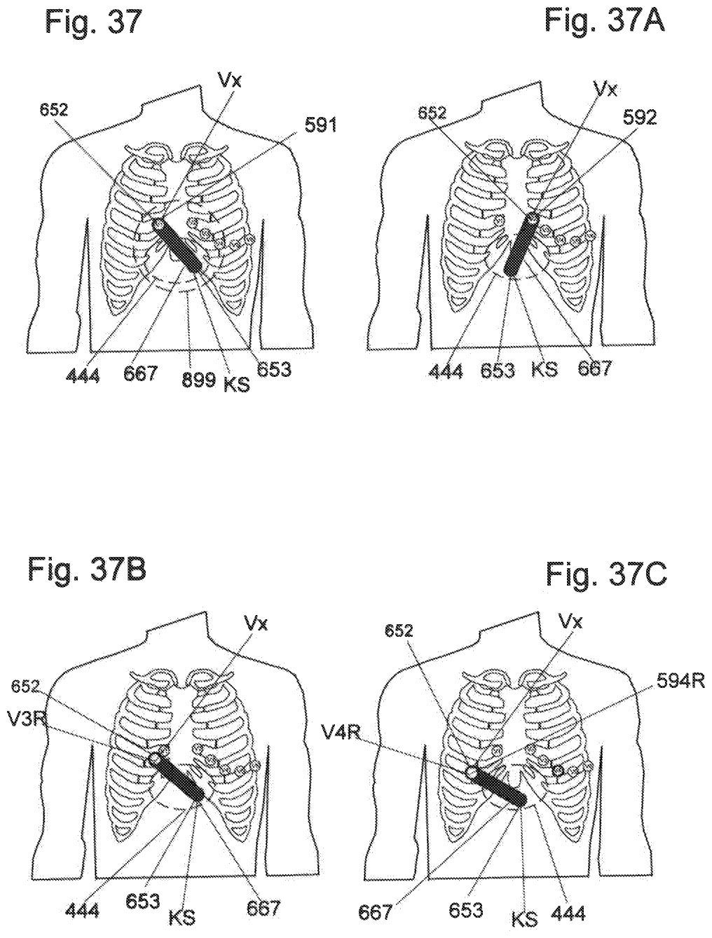

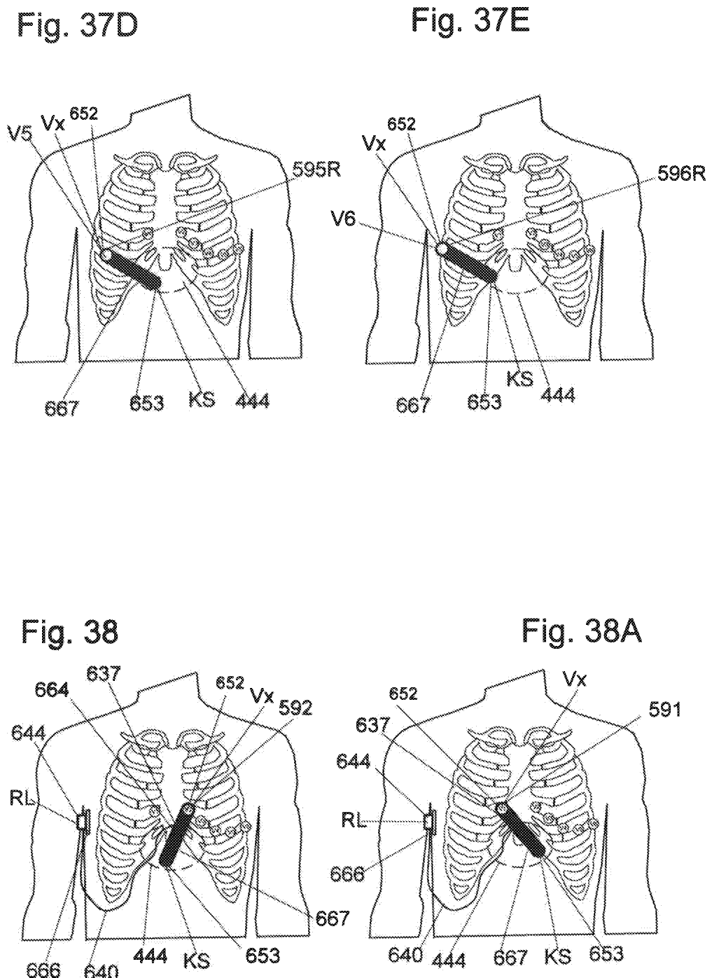

FIG. 37 shows location of the base 667 with the electrodes 652 and 653 and the monitor 668 on the chest at the position 899 of the Kranz's terminal (KS) used for sensing of the chest leads opposite to the Kranz's terminal. The switch 638 in FIG. 36 is at the B position for the Vx. The electrode 653 is placed in the area 444 of the Kranz's terminal and the electrode 652 is at the location 591 recommended for sensing of the lead V1. The position is used for sensing of the lead V1. In sensing of the left side leads, the position is the same as the one for sensing of the right side lead V2R. In this sensing, the reference, or the feedback is used, using the resistors 425 located on the base 667 shown in FIG. 36.

FIGS. 37A-37E show sequentially the positions of the electrode 652 for sensing of the leads V1R, which position is the same as the position for sensing of the lead V2, and then sequentially the positions for sensing of the leads V2R, which position is the same as the position for sensing of the lead V1, and further the position for V3R. to V6R.

FIG. 38 shows sensing using the base 667 with RL reference. In this case, all chest leads are sensed so as in case of the embodiment shown in FIG. 37, but the electrode 644 is used as reference, which electrode is located on the “small board 666 for the reference electrode” and is connected to the base 667 via the connector 664. Preferably, the monitored person inserts this electrode under its garment into the armpit. The artificial reference is disconnected by the switch 644 in the position C. This Figure shows the position for sensing of the lead V1R, which position is identical to that for sensing of the lead V2.

FIG. 38A shows sensing of the chest leads using the base 667 with the reference RL for sensing of the V2R lead, which position is identical to that for sensing of the lead V1. Additional leads V3R to V6R are sensed similarly to what is shown in FIGS. 37B to 37E.

FIG. 39 shows the principle of sensing of the chest leads by relocation of the base 667 to the position 900 with the small board by means of the base 667 using the electrodes 653 for the Kranz's terminal/LL, the electrodes 652 for reference in the abdominal area, and the electrodes 639 Vx on the small board 654 of the electrode on a cable connected via the connector 637 and by means of a single-channel monitor. The electrode 653 for the Kranz's terminal/LL is placed in the area 444 of the Kranz's terminal. The electrode 652 Vx/REF is placed downwards in the area of the abdomen. The electrode 639 for Vx on the small board 654 of the electrode on the cable is placed at points recommended for sensing of the chest leads V1 to V6 and V1R to V9R.

Switch 638 and switch 643 are in position C.

FIG. 39A shows using of a chair backrest for pressing of the electrode 639 against the back to sense leads V7R to V9R.

FIG. 39B shows positions for sensing of chest leads using the abdominal reference of FIG. 39, namely of leads V1 to V6 and V1R to V6R by sequential relocation of the electrode 636.

FIG. 39C shows the positions for sensing of the chest leads using the abdominal reference as shown in FIG. 39, namely of the rear leads, i.e. of V7 to V8.

FIG. 39D shows positions of the small board 641 for a two-channel monitor for sensing of chest leads using abdominal reference. This embodiment makes it possible to sense the following pairs of leads at the same time: V1 and V2, V1R and V2R, V3 and V4, V5 and V6, V3R and V4R, V5R and V6R, and further (V7 and V8) or (V8 and V9). The monitored person holds the base 420 pressed against the area of the Kranz's terminal and the abdomen with one hand and with the other hand it places the small board 641 to places as shown in this Figure to sense the respective pair of leads.

FIG. 39E shows the position of the small board 641 for sensing of the leads V5 and V6.

FIG. 39F shows the position of the small board 641 for sensing of the leads V7 and V8.

FIG. 39G shows sensing by means of the base 420 of the universal monitor in conjunction with the small board 725 of three chest electrodes 728, preferably formed by electrodes 639, 660, and 709. The sensing procedure is similar to that in FIG. 39D, when the base 420 is held in the left hand so that the electrode 653 abuts the area 444 of the Kranz's terminal and the electrode 652 abuts the central area of the abdomen. A small board 725 of three chest electrodes that is attached to the areas recommended for sensing V4R+V5R+V6R, V4+V5+V6 and V7+V8+V9 is attached to the base 420 via a cable. The small board 725 of three chest electrodes comprises three electrodes and preferably it is adapted for bending approximately in one third at both ends to change the mutual position of the outside electrode and of the remaining two electrodes so that it is possible to adapt it to a shape suitable for the positions recommended for sensing of the V1+V2+V3 and V1R+V2R+V3R. All V leads can then be sensed by five attached small boards 725.

FIG. 39H shows application of the small board 725 of the three chest electrodes to the site recommended for sensing the back chest leads V7, V8 and V8.

FIG. 39J shows a modification of the small board 725 of the three chest electrodes to change the shape by deviating the end portion with the electrode so as to obtain a geometry suitable for sensing of the chest leads V1, V2, V3 and V1R, V2R and V3R. This Figure shows lowering of the left part of the small board 725 for sensing of V1R, V2R and V3R. The board can be fixed in the lowered position.

To scan leads V1, V2 and V3, the left part returns to the straightened position and the right part is tilted.

This position is shown in FIG. 39G for sensing of V1 to V3.

FIG. 40 shows a sensing unit 571 both for applying and carrying, for sensing of ECG, preferably, provided with a two-channel monitor, using an RL reference, wherein, preferably, the sensing unit is formed by a “base 620 for RL” which base is provided with a connecting element 560, preferably formed by connecting elements 486, in this case preferably formed by snap fasteners 487, which is used for electrical and mechanical connection of the monitor 459 with the electrodes 143, which electrodes are preferably formed by grouped electrodes 471, which are sensing signals of multiple leads simultaneously. The monitor has one output, which is interconnected with the electrode 143, which electrode is preferably formed by an “electrode 621 for RL” for injecting of a reference signal RL, and by inputs for an electrode 678 for back leads V7 to V9. FIG. 40 shows a variant where the signals from the electrodes are fed through an external “cable 679 of the base” running along the base 620. Alternatively, the signal from the electrodes 143 is routed by conductors through the base 620. The base 620 is provided with electrodes 143. preferably by those that are formed by electrodes 421 of the base, preferably formed by elongated large-area electrodes 471 having length of 20 to 120 mm, for sensing o grouped cardiac signals comprising ECG leads corresponding to spots on the body for the monitored persons, in which spots the large-area electrodes are attached. The electrode 471 on the left hand side of the chest senses the grouped leads V4 to V6 against the electrode 471 on the right side of the chest sensing the V4R to V6R. This produces a grouped signal V4 to V6 and V4R to V6R, which includes the ST section for all of these leads to reveal an elevation of the ST segment in the event of a heart attack. The ST segment ST of the back leads V7 to V9 is sensed by the electrode 678 by moving the base 620 horizontally, as it is shown in FIG. 40. The base 620 is put on the body and it is held by hand for a short-term sensing, or by means of the shaped holes 561 on both sides of the base 620, which base is preferably in the shape of a belt, the elastic portion 82 of the belt is attached, which provides encircling of the chest and pushes the base 620 to the chest for a long-time sensing.

Preferably, the base 620 is made of a flexible plastic belt to facilitate insertion under garment, such as a shirt or a T-shirt, along the left hand side of the chest up to the back leads V7 to V8. Alternatively, it is preferably made as a fabric belt with attached electrodes 143, to which a cover 681 is fastened, preferably one of some fabric, for example by gluing or sewing, which preferably covers the externally placed cables 679, and it is adapted for inserting of a strut 680 of the belt. Which strut is preferably used to facilitate insertion of the base 620 under the garment for the purpose to sense by means of the applied base held by hand and, preferably, by pressing of the electrode 471 for leads V7 to V9 so that the monitored person leans against a chair back rest, as it is shown in FIG. 39A.

In case of fixing of the base 620 to the chest by means of the resilient part 82, preferably the strut 680 is not used.

Preferably, the strut is used for the left hand side of the base, for insertion to the back leads V7 to V9.

FIG. 40A shows the initial position of the base 620 for the RL shown in FIG. 40.

One electrode 421 of the base senses the potential from the area recommended for sensing of leads V5R and V6R, and the other electrode 421 from the area for V5 and V6, i.e. symmetrically around the sternum. Thus, the potential difference between the so positioned collecting electrodes is sensed, wherein, the reference RL by means of the electrode 621 is used preferably.

FIG. 40B shows sensing after the base on chest has been moved to the left. This brings the probe 471 that sensed the V5R and V6R areas to the area 444 of the Kranz's terminal and the second collection probe to the V6 area. In this position of the base, the electrode 678 for the back leads is placed with its center in the location, which is recommended for sensing of V8. If a two-channel monitor is used, this monitor senses the collecting lead V5/V6 and the back collecting lead V8 against the Kranz's terminal.

FIG. 40C shows sensing after moving to the right from the home position. In this position, the combined signal from the V5R and V6R areas is sensed against the Kranz's terminal

FIG. 40D shows a principal block diagram of sensing shown in FIG. 40A.

FIG. 40E shows a principal block diagram of sensing shown in FIG. 40B.

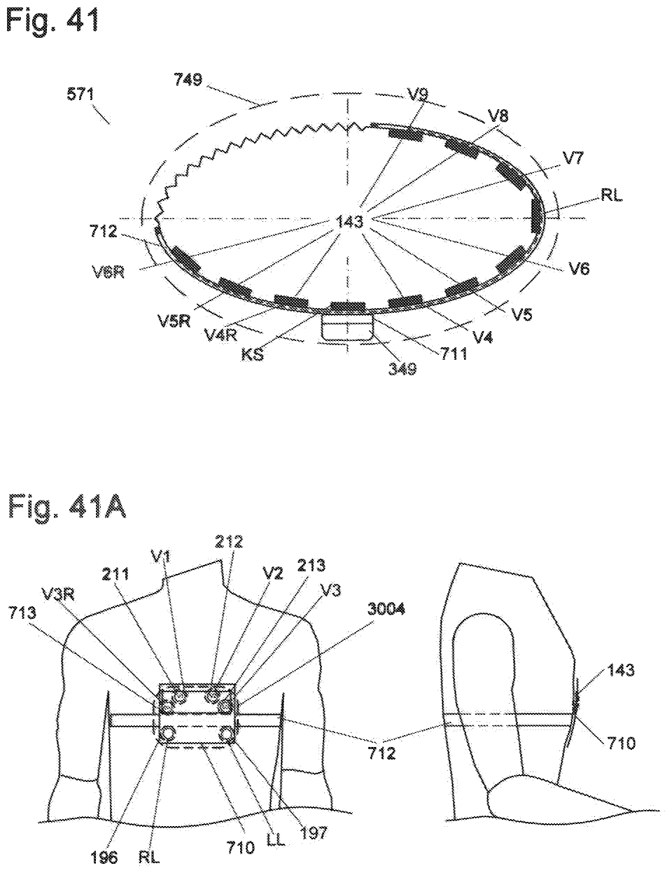

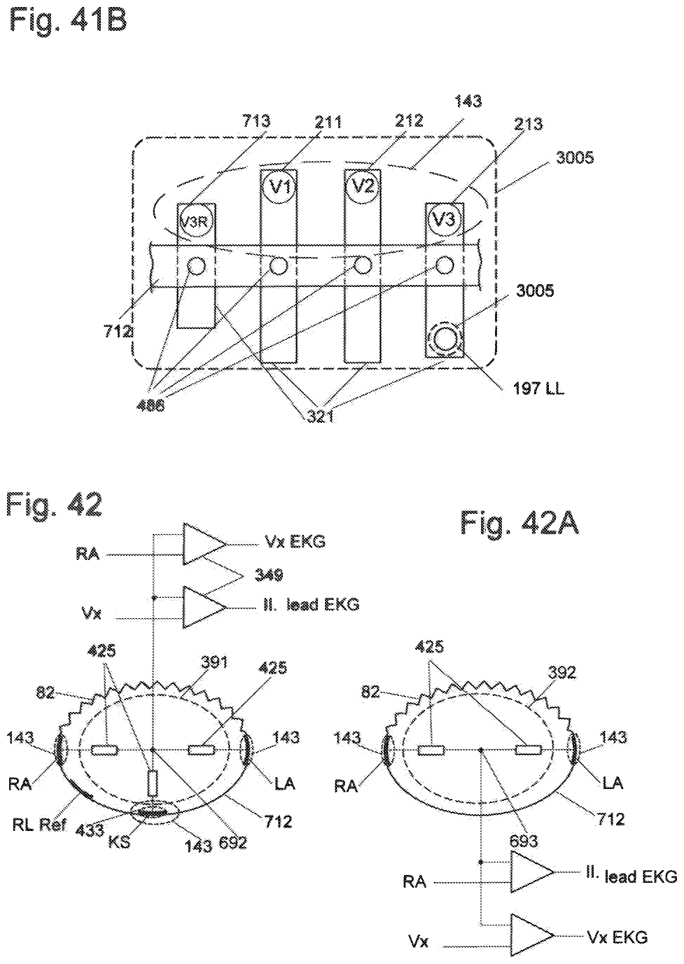

FIG. 41 shows the principle of the sensing unit 571, preferably in the form of a chest belt for sensing of an ECG using 2, 3, or up to nine-channel monitor 349 or, in the case of a greater number of electrodes 143, also by a multi-channel monitor 349, using a Kranz's terminal.

Preferably, the sensing unit is formed by a complete base 712 which base is attached to the chest or, upon attachment of the resilient portion 82, it encloses the chest. The base is provided with electrodes 143 so that they abut to the chest and back at the spots recommended for sensing of the V6R, V5R, V4R, V4, V5, V6, V7, V8 and V9. Further, one electrode 143 for the Kranz's terminal is in the middle of the chest and one electrode 143 for pro the RL is on the left side of the body. Thus, the sensing unit 571 can sense all potentials at the recommended sensing spots that, which spots are in one line. The electrodes 143 are led to the interconnection field 711. The sensing by means of the sensing units 571, preferably formed by the base 712, is designed to be the best possible using only the electrodes located on the sensing unit in the form of a belt. Therefore, sensing of the leads I, II and also of the leads V1/V2R, V2/V1R, V3, V3R is missing. If it is shown that these leads are required, an enlarging board 710 is attached to the base 712 to allow sensing of the V1/V2R, V2/V1R, V3, V3R leads of the electrode 197 LL, and simultaneously also connection of the electrodes to the monitor inputs. Alternatively, independent electrode segments 321 are used to sense these leads, which are flexible, connected by means the connecting elements 486. The monitor 349, the board 710 and the electrodes are brought into the interconnecting field 711, which field allows to bring the respective lead to the respective channel via the interconnecting field 711. In case the monitor 349 having fewer channels than what is the required number for the number of electrodes used, it is scanned sequentially using the number of electrodes that can be connected to the monitor, and further electrodes for the subsequent sensing are connected sequentially via the interconnecting field 711.

FIG. 41A shows the use of an enlarging board 710 in cooperation with a complete base 712. The board 710 includes four electrodes which electrodes, after connecting of the enlarging board 710 to the complete base 712 fit closely to the sites recommended for sensing, i.e. the electrode 211 for V1/V2R, 212 for V2/V1R, 213 for V3 and 713 for V3R. The board 710 is mounted centrally on the base 712 so that it extends beyond it, and preferably, it is slightly curved to press the electrodes 143 against the chest, as seen it is shown in detail “D”. The board 710 is usable for sensing on all models of sensing units 571 that are in the shape of a chest belt.

FIG. 41B shows the use of electrode segments 321 to supplement both sensing and leads V1/V2R, V2/V1R, V3, and V3R. The electrodes 143, preferably formed by the electrode 211 for V1/V2R, 212 for V2/V1R, 213 for V3, and 713 for V3R, and for LL, are fixed by means of a connecting element 486 to the complete base 712, and they are connected to the interconnecting field 711. For the sake of clarity, neither the interconnecting field 711 nor the monitor 349 are shown in this Figure.

This solution makes it possible to sense signals at points that are distant from the longitudinal axis of the base 712, and at the same time, since the electrode segments are flexible and suitably shaped, pressure of the electrodes 143 against the skin is ensured.

The segments 321 are applicable to all types of chest belt shaped sensing units 571.

FIG. 42 shows a method for obtaining of a reinforced Kranz's terminal 693 for a more accurate sensing than that with the pseudo-Kranz's terminal 693 by means of circuits for the reinforced Kranz's terminal, preferably formed by three resistors 425 that are connected between the electrodes and sense potentials of RA, LA, and the Kranz's terminal, used to obtain their a center. Individual chest leads are sensed against this center. Depending on the sensing means used, the resistors 425 are located in the base 712, the interconnecting field 711 or the monitor 349. The reinforced Kranz's terminals can be used to process chest leads on the monitor 349 by connecting the electrode 143 for Vx to the Vx lead; when the electrode 194 RA is connected to the reinforced Kranz's terminal the lead II ECG can be obtained.

FIG. 42A shows formation of the pseudo-Kranz's terminal 693 by conducting the RA and LA potentials through resistors 425 to one point of the Kranz's terminal 693, which terminal, similarly as the reinforced Kranz's terminal, can be used to sense ECG lead II when the electrode 194 for RA or the chest lead Vx is connected when the electrode 143 for Vx is connected.

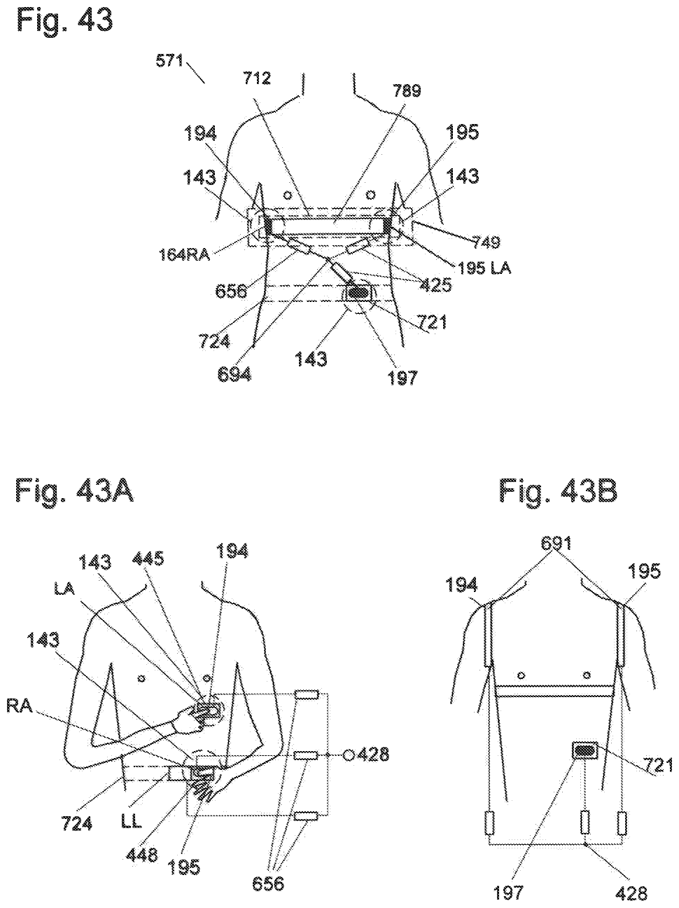

FIG. 43 shows the formation of a pseudo-Wilson's terminal 694 from potentials of the RA, LA obtained by electrodes 143, preferably formed by electrodes 164 RA and 195 LA of the base 712 that are placed on the body sides of the monitored person, and the potential LL obtained preferably by means of electrode 143, preferably formed by electrode 197 LL on the abdominal small board 721, which potentials are led to one point by resistors 425.

Preferably, the pseudo-Wilson's terminal is used in such a way that for all mentioned sensing against the Kranz's terminal, the potential of the Kranz's terminal is replaced by the potential of the pseudo-Wilson's terminal. Thereby a better signal can be achieved. The advantage of the Kranz's terminal over the pseudo-Wilson's terminal consists in that all electrodes 143 are situated in one plane, preferably in the bases 789 in the form of a belt. The advantage of the pseudo-Wilson's terminal over the Wilson's terminal is that only one electrode 197 for LL is located outside the base 789 shaped as belt. The belt shaped base 789 is preferably formed by one of the following options: an extended base 624, a base 620 for RL, a simplified base 720, and a multi-electrode base 630.

FIG. 43A shows an example of sensing with a fully-fledged Wilson's terminal 428 from the left and right arms, LA, RA, and the left leg LL. In this embodiment shown in FIG. 8, the monitored person holds the base 445 of the multi-lead ECG monitor by pressing the electrode 143 for the right arm RA with his/her right hand, to press it at best against the chest. The small two-electrode board 448 is held with left hand by pressing the left-arm LA electrode 143 LA, whereby the two electrodes RL, LL are pressed to the abdomen.

FIG. 43B shows another example of obtaining a Wilson's terminal 428 from the RA, LA using a shoulder strap 691 provided with a an electrode and the LL electrode 197 on an abdominal small board 721.

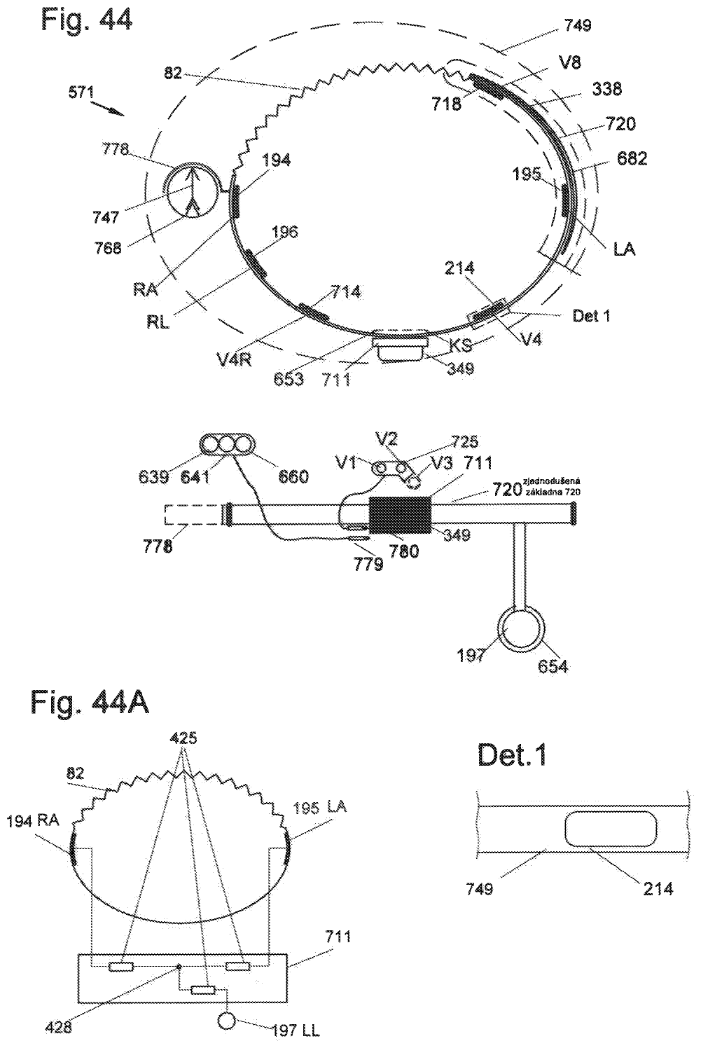

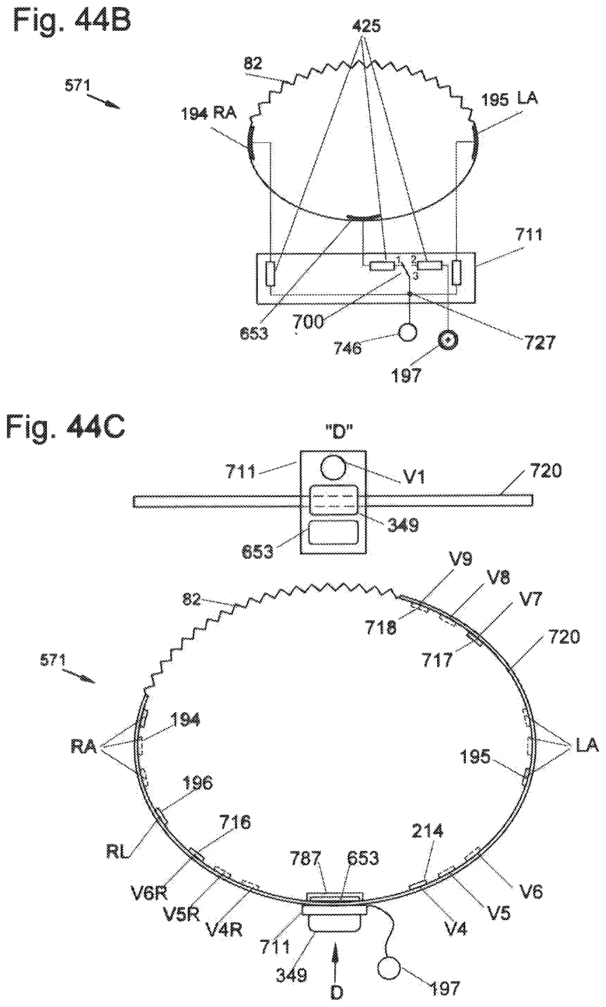

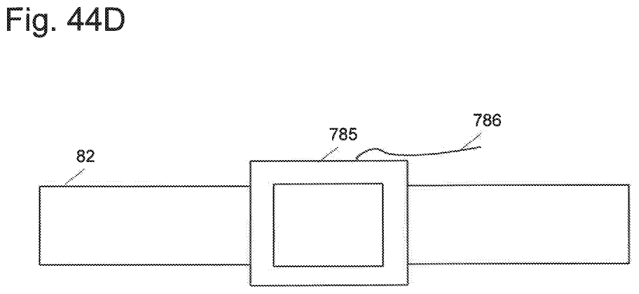

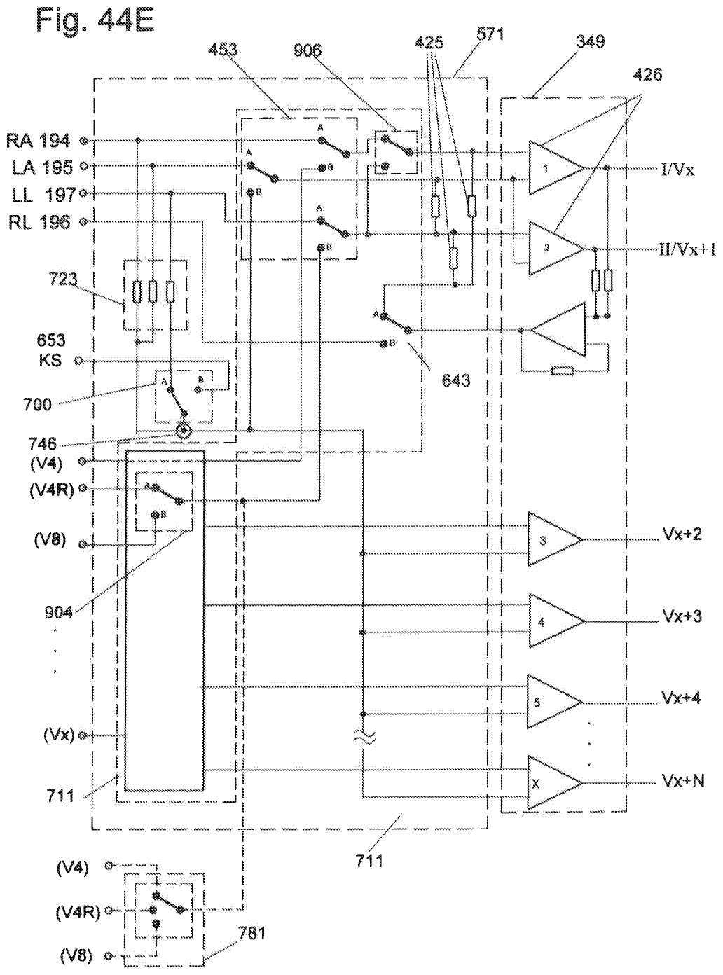

FIG. 44 shows the chest belt 749, preferably formed of a simplified base 720, which is used for sensing of optional chest leads, in this example of the V4R, V4 and V8 leads, against a pseudo-Wilson's terminal by means of the electrodes 714 for V4R, 214 for V4 and 718 for V8R. These leads were chosen because they are important for capturing of the ST segment elevation. In order to obtain a stronger cardiac signal for displaying a better image during physical movement of the monitored person, the electrodes 714, 214, 718 are provided with a large-area as shown in the Detail 1, wherein the area is at least 20×30 mm or more, up to 20×100 mm.

Preferably, the chest belt 749 is formed by the base 720, or by any other base, for example by the extended base 624, it is provided with an extension portion 682 of the base, preferably formed by a brace 338, which brace allows easy insertion of the base by the right hand under the clothes, such as under a shirt, without undressing, because the base does not bend and holds the direction of insertion. Preferably, the chest belt 749 is provided with an extension 778 for an arm, which extension allows the monitored person, despite the cloths, to exert a greater pressure of the base towards the body, and thereby to improve the contact of the electrodes with the skin. After insertion of the extended portion 682, the subject monitored at first rests thoroughly with the back against the chair backrest, exerting thereby pressure to the base at the spot of the back electrode 718 for the V8 and prevents sliding away of the extended portion 682 from the back. Subsequently, by moving the right arm backwards in the direction indicated by the arrow 747, the arm pressure acts on the extension 778 for said arm, whereby sufficient additional pressure of the base to the body is exerted, and thus of all electrodes on the base.

Alternatively, instead of the extension 778, it is possible to switch the elastic part 82 of the belt for pressing of the chest belt 749 against the body. With the extended part 82 long enough, the chest belt 749 can be inserted around the back with the right hand and the left hand under the shirt, from the end of the extended portion 682 to grip the put on elastic portion 82, and to fix it to the beginning of the belt and after it has been rotated back into the operational position to have the belt fixed without undressing of the shirt.

The preferred embodiment is such, where the interconnecting field 711 also comprises a socket connector 780 with a switch. After connection of the pin connector 779 into the socket connector 780, the interconnecting field 711 is switched from the electrodes on the chest belt 749 to the electrodes on the small board 654 or 725, for example electrodes 714 for V4R and electrodes 718 for V8 are switched to the electrode 639 for Vx and the electrode 660 for Vx+1, by which it is possible to sense the leads V1, V2, V3, V3R and other selected chest leads after attaching the small boards 654 or 725. The small board 654 or 725 for sensing of chest leads is attached to the base by means of the pin connector 779.

In an alternative solution, the small board 654 or 725 is connected by a connector and switching of electrodes is carried out by a switch or by an interconnecting field 711.

FIG. 44A shows a pseudo-Wilson's terminal which is formed of the RA, LA signals that are connected via resistors 425, sensed by electrodes 194 and 195, located on the chest belt 749, preferably formed by the base 720 so that they abut against the sides of the monitored subject's chest, and of the LL signal, which is introduced from the LL electrode 197 for LL on a cable, which electrode is placed in the left abdominal area and held is held by hand, or preferably, it is inserted under the belt, or it is fastened with a waist belt 724.

The electrode 197 for LL is connected to the interconnecting field 711, which field is used to connect the selected electrodes according to needs to the monitor 349. The electrode 196 for RL is located on the chest belt 749, preferably formed by a simplified base 720, and is preferably used for reference. Alternatively, the pseudo-Wilson's terminal is switchable by the KS/WS (Kranz's terminal/Wilson's terminal) switch 700 shown in FIG. 44B.

In a block diagram FIG. 44B shows the principle of switching between sensing using a Wilson's terminal and using a Kranz's terminal. The Kranz's terminal/Wilson's terminal switch 700 switches from position 1 to position 3, thereby switching the signal from the Kranz's terminal electrode 653, which signal is fed through the resistor 425 to the reinforced Kranz's terminal, which terminal is obtained from the other signals LA, RA by means of the resistors 425, to the electrode 197 for LL, thereby in point 727 of the Kranz's terminal/Wilson's terminal switch switches from the reinforced Kranz's terminal to the Wilson's terminal. By switching of the Kranz's terminal/Wilson's terminal switch 700 to position 2, both the Kranz's terminal electrode 653 and the electrode 197 for LL are disconnected and the terminal 746 will exhibit the pseudo-Kranz's terminal signal which signal corresponds to the signal at terminal 693 described in FIG. 42A. The switching can be implemented in the interconnecting field 711.

FIG. 44C shows the use of a sensing unit 571 preferably formed by the chest belt 749, which belt is preferably formed by a simplified base 720 for sensing additional chest leads by slight turning of the base 720. In this embodiment, the base is provided with suitable electrodes for slight turning, in this example instead of the electrode 714 for V4R, the electrode 716 for V6R is used and instead of the electrode 718 for V8, the electrode 717 for V7 is used.

In the basic position of the base 720, the leads V6R, V4 and V7 are sensed. After the first slight turning of the base 720 to the left relative to body of the monitored person by approximately 3 to 4 cm, the leads V5R, V5 and V8 are measured. After the second slight turning by approximately 3 to 4 cm, the leads V4R, V6 and V9 are measured.

Although the electrodes 194 for RA and 195 for LA move along the sides of the monitored person, their signals are still equally useful for obtaining the RA and LA signals with sufficient accuracy.

Preferably, the electrode 653 is covered by a transverse strip 787 fitted with two electrodes, one for Vx and one for Kranz's terminal/LL. The cross belt 787 is snapped onto the base and is connected to the connecting field 711. The electrode Vx senses the lead V1 (V2R) in the basic position. The Kranz's terminal/LL electrode is used for LL. After the first slight turning to the left, from the point of view of the monitored person, the electrode Vx senses the lead V2 (V1R) and after the second slight turning the lead V3. This significantly expands the sensing options. Optionally, it is possible to use all models of Kranz's terminal or Wilson's terminal that are shown in FIG. 42.

FIG. 44D shows a slide on electrode 786. These electrodes are slidden on an elastic portion 82 of the belt which portion presses them against the subject's chest. The advantage of such an electrode is the possibility to place it as accurately as possible in the position recommended for sensing a specific chest lead. The contact surface of the electrode is led out by the electrode terminal 786, which is connected to the connection field 711.

FIG. 44E shows a block diagram of a monitor 349 connection, including the interconnecting field 711, which switches the electrodes 143 to the input circuit 349 of the monitor for various sensing units 571. Its function is evident from the example for a simplified base 720 and a dual-channel monitor. The signals RA, LA, LL are alternatively fed to the inputs of the operational amplifiers 426 by the switch 722 in the position A for sensing of leads I and II and for calculating the leads III, aVR, aVL and aVF, and after switching of the switch 722 to the position B, the signals Vx, in this case with switch 904 V4R/V8, is switched to V8 sensing. When using a three-channel monitor, V4, V4R and V8 are sensed simultaneously. For a single-channel monitor, the 784 V4/V4R/V8 switch shown as the V2 interconnecting field 902 is used to sequentially sense V4, V4R, and V8.

This connection allows to work with the reference RL supplied to the body of the monitored person in the position B of the reference switch 643, or with an artificial reference, supplied via resistors 425 to the inputs of said operational amplifiers in position A of the “reference switch 643”. The signals Vx are measured against the Wilson's terminal, which terminal is generated by the Wilson's terminal circuit 723 in position A of the switch 700 Kranz's terminal/Wilson's terminal. In the position B, electrode 653 for the Kranz's terminal/LL is connected for sensing by means of the Kranz's terminal.

Using the five-channel monitor 349 by means of the simplified base 720 shown in FIG. 44, leads I and II can be sensed in position A of the switch 722 (I, II/Vx, Vx+1) and further simultaneously always trios of chest leads, always against the Wilson's terminal, V6R, V4, V7, further V5R, V5, V8 and further V4R, V6 and V9 using operational amplifiers 3 to 5. In case of switching of the switch 722 to position B, it is possible to sense simultaneously five selected chest leads simultaneously.

The signals from the individual electrodes are fed to the interconnecting field 711, which fields makes it possible to connect any input to any output, and thus to the monitor channel. The base 720 according to the example is designed to sense five leads simultaneously, and thus to use a five-channel monitor.

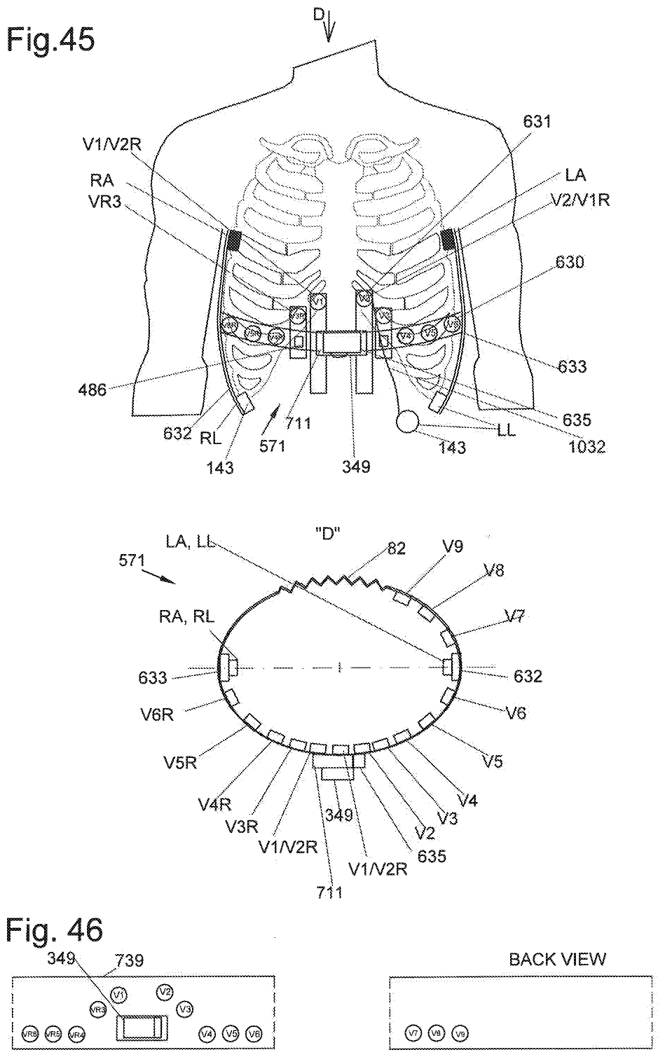

In using of the multi-electrode base 630 shown in FIG. 45, it is possible apply signals from the electrodes for sensing at points RA, LA, RL, LL, V6R, V5R, V4R, V3R, V1/V2R, V2/V1R, V3, V4, V5, V6, V7, V8, V9. Therefore, leads I, II, V6R, V5R, V4R, V3R, V2R, V1R, V1, V2, V3, V4, V5, V6, V7, V8, V9 can be scanned at once, while the other 4 leads III, aVR, aVF, and aVL can be calculated, i.e. 21 leads altogether.

For simultaneous sensing of 21 leads, an interconnecting field 711 is required with n=18 inputs, for 15 chest leads and for three signals LA, RA, LL.

Preferably, the interconnecting field 711 is modular and it allows to connect a monitor or monitors in accordance with the required number of the simultaneously sensed channels, and this from 1 to 17. One multi-channel monitor or multiple monitors, for example three eight-channel monitors can be mounted side by side. At the same time, it allows sequential sensing with switching over, as it was elucidated in the examples of use in the case of the base 720.

The switch 700 Kranz's terminal/Wilson's terminal, which switches the electrode 653 for the Kranz's terminal/LL to electrode 197 LL, is used to select sensing against the Kranz's terminal or the Wilson's terminal. The Kranz's terminal has the advantage that all electrodes are on the sensor unit 571, which unit is formed preferably by a simplified base 720, preferably in the form of a belt. In the case of a pseudo-Kranz's terminal when the electrode 143 for the Kranz's terminal is not applied, for a transfer from measuring by means the Kranz's terminal to measurement by means of the Wilson's terminal measurement is enough to connect the electrode 143 for LL.

FIG. 45 shows the use of a sensing unit 571 which unit is formed preferably by a multi-electrode base 630 for sensing of up to 18-lead-ECG using a Wilson's terminal. The multi-electrode base is based on the concept of a complete base 712 shown in FIG. 41, but it is substantially expanded. The electrodes 143 are placed directly on it so that they abut to the sites that are recommended for sensing of V4, V5, V6, V7, V8, V9, V4R, V5R, V6R, and alternatively, also the electrodes 143 for sensing of spare signals RA, LA.

All these points lie approximately on a line running around the chest of a standing person at the same height. The lead signals outside this connecting line, i. e. the V1/V2R, V2/V1R, V3, V3R, are sensed by means of electrodes 143 on the transverse strips 631, and further, signals/leads RA, RL, LA and LL are sensed by means of the side strips, namely the side strip 632 senses signals LA and LL and the second side strip 632 senses signals RA and RL.

Alternatively, a wide chest belt 739, shown in FIG. 46, is used.

FIG. 46 shows the use of a wide chest belt 739, the upper line 636 of which is indicated in the Figure. Then, the electrodes V1/V2R, V2/V1R, V3, and V3R are part of the belt and no cross strips 631 with electrodes are needed.

All electrodes 143 are connected to a multiple switch 635 of leads, which is interconnected with a connector field 634. A multi-channel monitor, an eight-channel monitor 470, or even a multi-channel monitor can be connected to the connector field 634 via a connector. Depending on the connected monitor or the ECG device and on the demand for sensed leads, such connection can be made by means of the 635 of leads. Preferably, this multi-switch is remote controllable.

In full assembly, this arrangement allows immediate switching between a standard 12-lead ECG sensing to sensing of reverse leads, or to sensing of rear leads.

Preferably, the side strips 632 are removable and/or pivotally connected at the junction with the multi-electrode base 630 to minimize dimensions during transport or storage.

In the case of removing the strips 632, preferably, instead of the electrodes 143 for LA and RA, electrodes 194 for the spare signals L A′ and RA′ are preferably used. Preferably, an electrode 143 located in the left abdominal region and connected by a cable 1032 is used for LL,

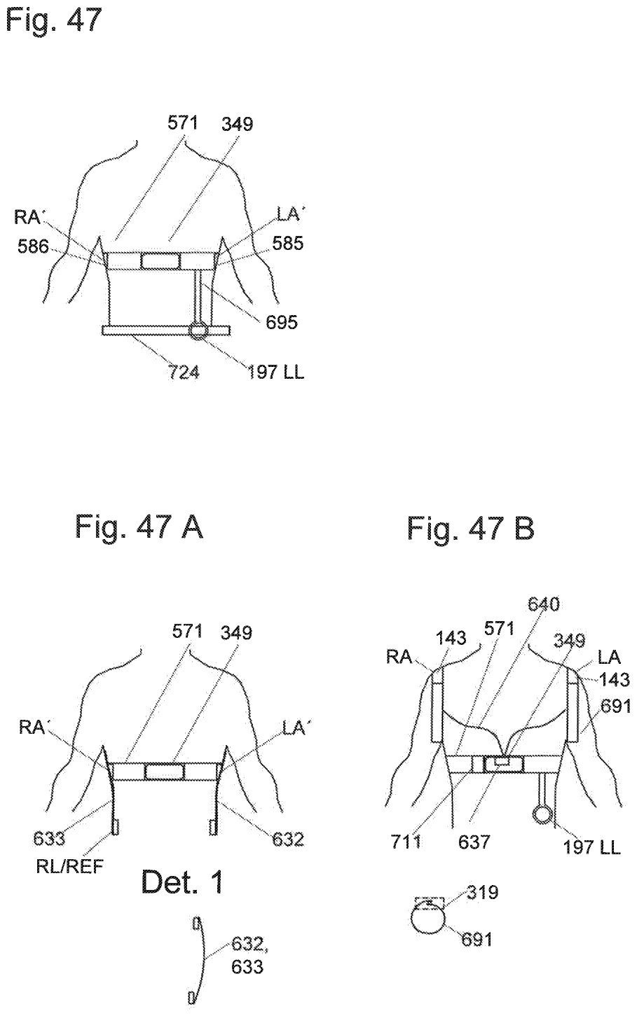

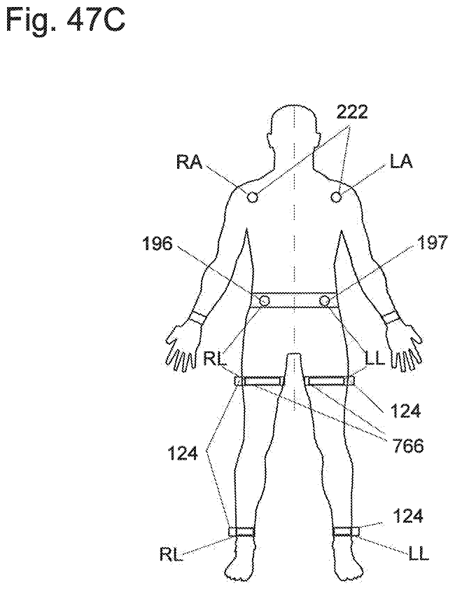

FIG. 47 and FIGS. 47A-47E illustrate clearly the methods of obtaining potentials for a Wilson's terminal 428 using electrodes 143, preferably formed by electrodes 686 for potentials RA, LA and LL, consisting of the electrode 197 for LLa, of electrodes 687 for potentials from the left and the right arms, preferably formed by electrodes 195 for the LA and 194 for the RA, or the electrodes 585.

In this example, the electrode 196 for RL is located on the abdominal belt 724, but it may be located also elsewhere, for example on the sensing unit 571 having the shape of a belt or of a small board.

FIG. 47 shows electrodes 586 RA′ and 585 LA′, which are located for the sake of simplicity of the attachment on the sides of the strip-shaped sensing unit 571, preferably they are formed by a base 720 or a multi-electrode base 630 located at the lower edge of the sternum.

The signals obtained here do not correspond exactly to the signals sensed from the electrodes 194 RA and 195 LA at the recommended sites on the left and right arms, but the small inaccuracy is balanced by the simplicity of attaching the electrodes 586, 585, which are located directly on the strip-shaped sensing unit 571.

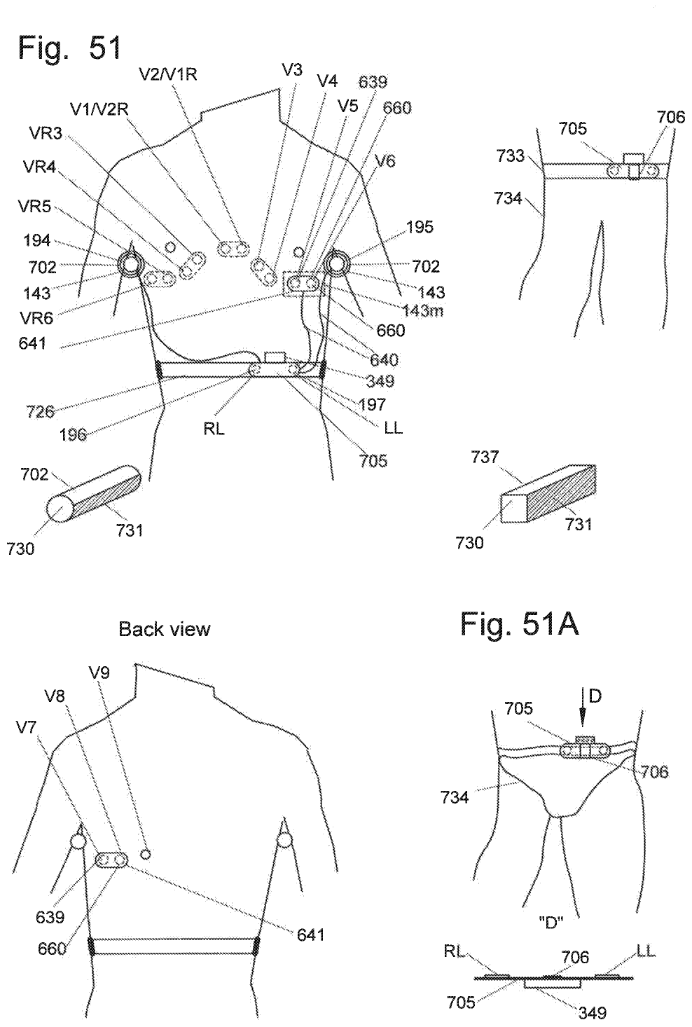

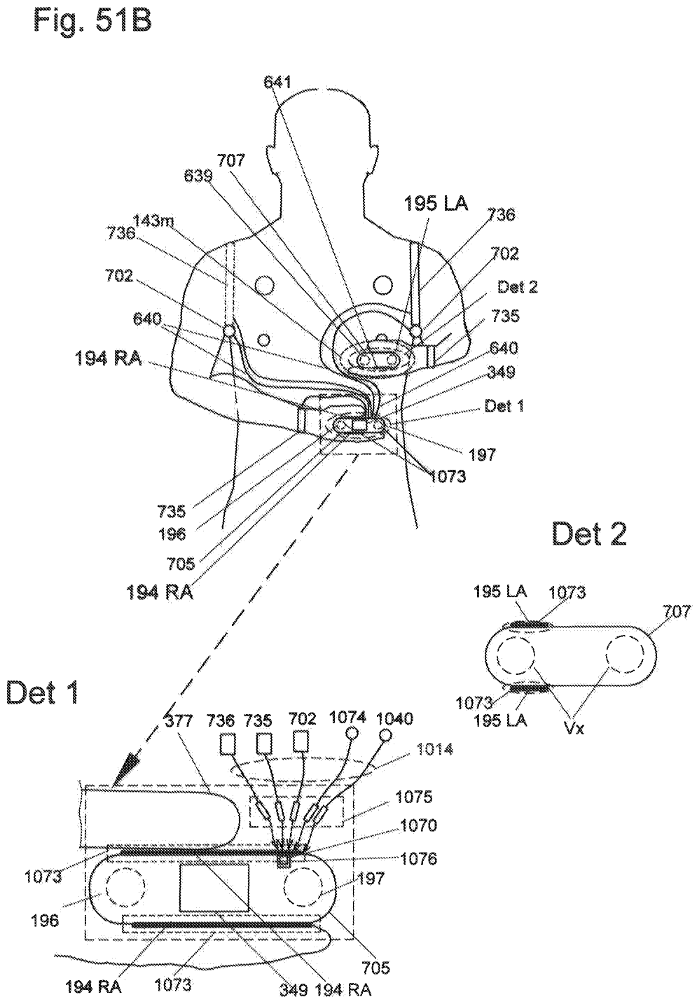

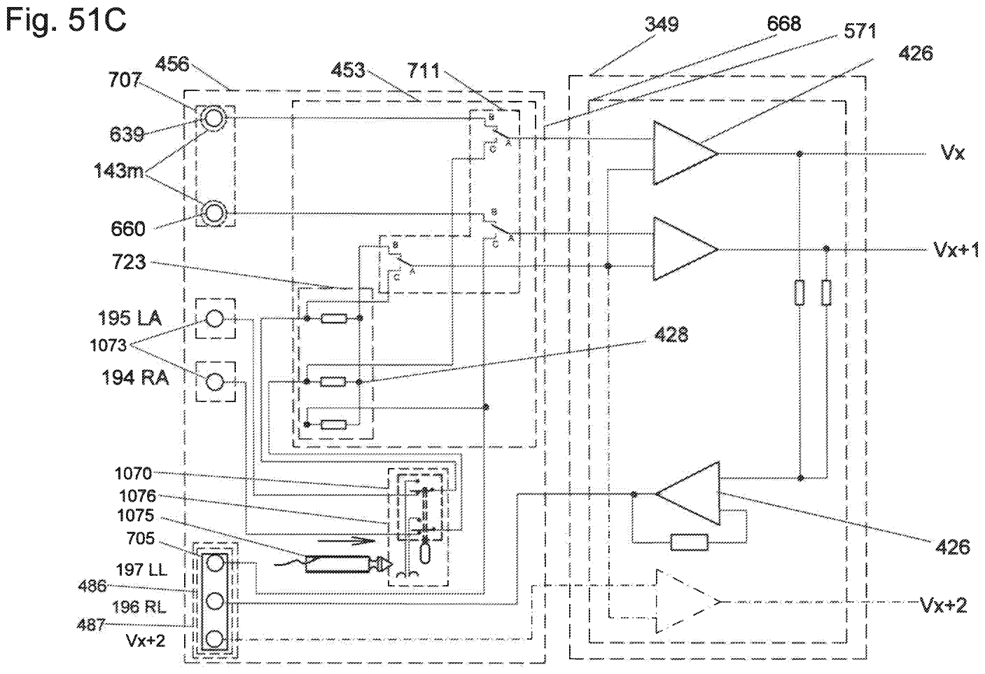

The electrode 143 formed by the electrode 197 for LL is placed on the abdominal electrode strip 695, which is connected electrically by the abdominal electrode strip 695, preferably removable to the base 571, and this arrangement allows to place the electrode 143, preferably formed by the electrode 197, to the spot recommended for sensing of LL in the left abdomen area. The belt-shaped sensing unit 571 is attached to the chest by the resilient portion 82 of the belt. The electrode 197 LL is attached preferably to the abdomen by the abdominal belt 724, or it is inserted, for example, under the panties 734, as it is shown in FIG. 51A.

FIG. 47A shows the use of side strips 632 to position electrodes 143 to obtain potentials RA, LA, LL connected mechanically and electrically, preferably removable on a sensor unit 571, preferably in the form of a strip by a connecting element 696 by which they are connected to a monitor 349. Preferably, the side strips 632, 633 are of resilient material, slightly bent, as it is shown in Detail 1, so that in the state of pressure by the belt-shaped base 571 they abut against the ends where the electrodes 686 are located on the body.

FIG. 47B shows the use of shoulder straps 691 with electrodes 687 for RA and LA. The shoulder straps 691 are connected by a cable 640 and a connector 740 to the monitor 349, preferably via an interconnecting field 711. After wrapping around the shoulder, the straps 691 are secured preferably with Velcro or they are made of a flexible material for sliding them on an arm. Preferably, they are of a conductive material, preferably formed by metal boards, which are attracted to each other by a spring mechanism and form electrodes 689 with their entire surface.

FIG. 47C shows the acquisition of potentials RA, LA and LL using common methods, i.e. by terminals 124 on limbs and/or by glued electrodes 222. An alternative acquisition of potentials for RA, LA is by hand contact with electrodes 194, 449 shown alternatively in FIG. 8 or by means of the voluminous electrodes. 702 shown, for example, in FIG. 50D.

In this example, the electrode 197 LL is located on straps 766 placed on the thigh or the ankle. The reference signal RL is either produced artificially in the monitor as shown in FIG. 44E, or it is applied only by the electrode RL 196 on the sensor unit 571, preferably in the form of a belt or a strip 776, or on terminals 124, or they are glued. The electrode 196 RL can be placed at suitable sites shown in the Figures of this patent specification.

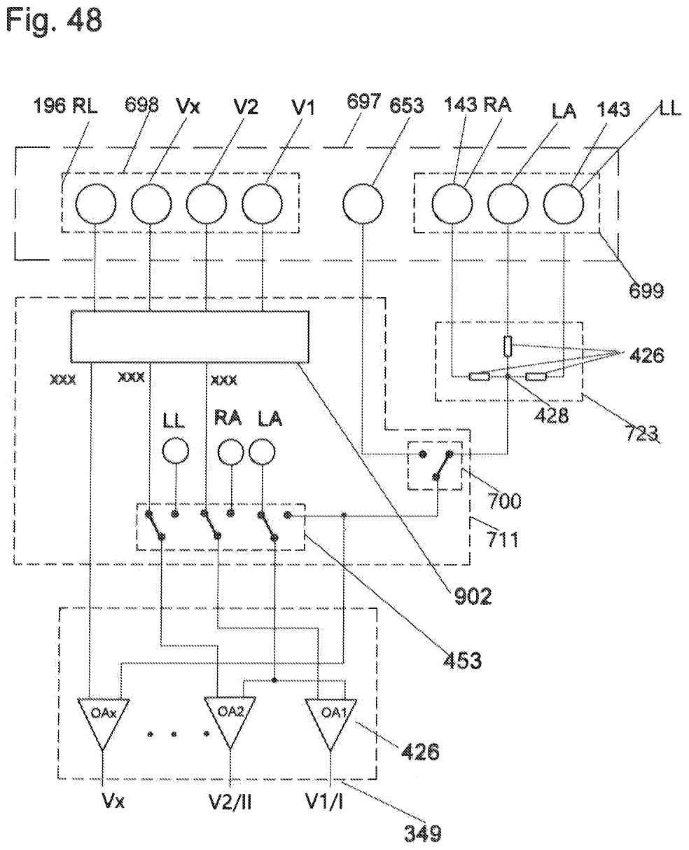

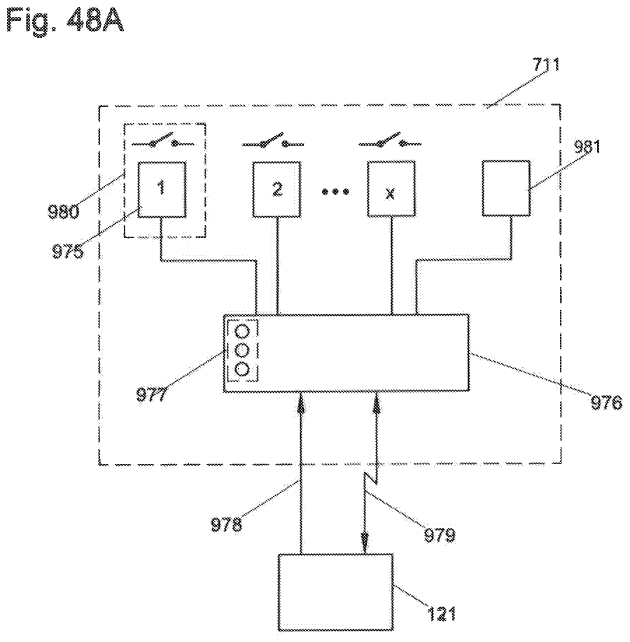

FIG. 48 shows an interconnecting field 711 connecting to the input circuits of the monitor 349, preferably consisting of operational amplifiers 426, electrodes 143, preferably formed by an electrode set 697, which set consists of a set 698 of chest lead electrodes from a set 699 of electrodes for the Wilson's terminal, and in case, no artificial reference is used inside the monitor 349, to the electrode 196 RL.

In this example, the interconnecting field 711 consists of an interconnecting field 902 for Vx, with which field the selected electrodes can be connected to the inputs of the operational amplifiers 426 of the monitor 349.

Further, the interconnecting field 711 consists of a switch 700 of the Kranz's terminal/Wilson's terminal, which can be used to switch the Wilson's terminal 428 or the electrode 653 for the Kranz's terminal to the input of the operational amplifiers, and so switch the sensing by the Kranz's terminal to sensing by the Wilson's terminal. Further, the interconnecting field 711 consists of a switch 453, which switch can be used to switch the inputs of the two operational amplifiers 426 from sensing of the Vx leads to sensing of the I and II leads for a two-channel monitor 349. In case of a single-channel monitor 349, the LL/RA switch 454 is used as it is shown in FIG. 11. The interconnecting field is formed by manual switches as it is shown in FIG. 48, 44D, 11 or 50A, for example.

Alternatively, instead of the switch 700, the monitor is switched from the Kranz's terminal sensing mode to the Wilson's terminal sensing mode by relocating it from the Kranz's terminal sensor unit 571, formed preferably by the base 667 of FIG. 35 to the Wilson's terminal sensor unit 571, preferably formed by the base 705 of FIG. 51. The set 699 of electrodes for the Wilson's terminal consists of three electrodes 143 for RA, LA and LL. The chest lead electrode set 698 is useful for sensing of up to 15 chest leads, V1 to V6, V1R to V6R, V7 to V9, wherein it is valid that V1R=V2 and V2R=V1. With the help of the set 699 of electrodes for the Wilson's terminal, i. e. of the electrodes for RA, LA and LL, leads I, II can be sensed and leads III, aVR, aVF, and aVL can be calculated, thus obtaining up to 21-lead ECG by sensing of signals from 15 electrodes 143 using a two-channel monitor 349 shown in the block diagram in FIG. 44 E, to which the interconnecting field 711 sequentially switches the respective electrodes.

It is possible to use a multi-channel monitor, thus reducing the number of switches carried out by the interconnection field 711. A 17-channel monitor would sense all 21 leads at once. In this case, the interconnected field 711 would not be needed. For a 12-lead ECG, the 8-channel monitor 349 shown in FIG. 23 is sufficient for simultaneous sensing.

In the case of the single-channel monitor 349, the electrodes 143 for the RA and RL signals also need to be switched by the switch 454, as it is shown in FIG. 11.

FIG. 48A shows an alternative embodiment of the switches of the interconnecting field 711 by means of switching elements 980 formed by relays 975 controlled by a control unit 976 of the interconnecting field, preferably formed by a control unit of the monitor 349, which unit is controlled preferably by pushbuttons 977 of the interconnecting field.

The advantage is that for certain sensing that require to switch more switches, it is sufficient to press one pushbutton for the respective sensing, which pushbutton switches more respective relays, and thus the switching it simplified.

Another advantage is that the relays 975 are controlled by a control unit 976, which can be connected by a wired connection 978 or a wireless connection 979 to the cooperating units 121 of FIG. 11, by which connections they can be controlled remotely, and thus selected sensing of the required leads, preferably sensing by means of the Kranz's terminal or the Wilson's terminal. Advantageously, the switching elements 980 may be formed by electronic switches 981.

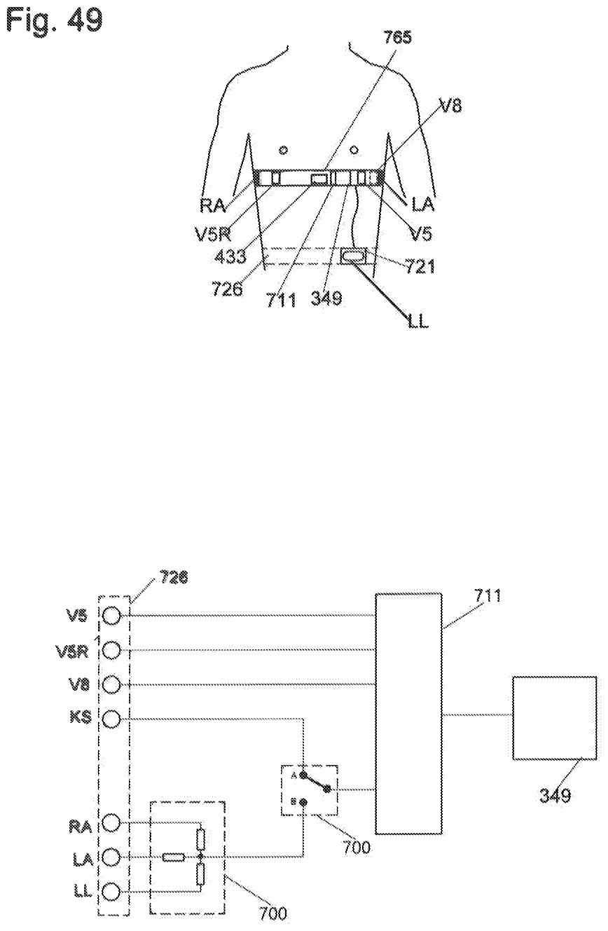

FIG. 49 shows switching between sensing with the Kranz's terminal and sensing with the Wilson's terminal 428 on the example of a sensing unit 571, preferably formed by a base 765 in the shape of a belt. The electrodes 143 for V5, V5R, V8 for the Kranz's terminal base 765 are connected preferably to the interconnecting field 711, wherein, the electrode 143, preferably formed by the electrode 418 of the base for the Kranz's terminal area is connected to the Kranz's terminal/Wilson's terminal switch 700.

Also the signals of the electrodes 143 for RA, LA are transmitted from the base 765 to the circuits 723 of the Wilson's terminal 428, together with the signal of the electrode 143 for LL from the small board 765 and the Wilson's terminal 428 signal is fed to the interconnecting field 711 to the Kranz's terminal/Wilson's terminal switch 700.

On the Kranz's terminal/Wilson's terminal switch 700, it is switched between the Kranz's terminal and the Wilson's terminal. The interconnecting field 711 switches the respective electrodes optionally to the monitor 349. The Kranz's terminal or the Wilson's terminal output from the circuit is transmitted to the interconnecting field 711, which field allows to connect the selected monitors 349.

The abdominal small board 720 for sensing of LL is held by hand, or preferably it is inserted under a trouser belt or the belt 726 for LL.

FIG. 50 shows a mechanical embodiment, and FIG. 50A a block diagram of a modification of the sensing unit 571 preferably comprising a board 667 having the shape of a small board as it is shown in FIG. 35 for the Kranz's terminal with ability to be switched over to the Wilson's terminal using a “Kranz's terminal/Wilson's terminal switch 700”, which switch makes possible to switch the monitor 349 from the electrode 653 for the Kranz's terminal/LL to “Wilson's reference circuits 582” connected via the connector 701 for Wilson's terminal and the electrode 653 Kranz's terminal/LL is connected to sensing LL for the Wilson's terminal. The kinds of placing and connecting of electrodes for RA, LA, and LL that are used for Wilson's terminal circuits are shown, for example, in FIG. 56D, wherein, the electrodes 194 RA and 195 LA are connected via connector 701 for Wilson's terminal.

The electrode 652 is switched to the RL reference by the switch 638 Vx/REF and the chest leads are sensed by the electrodes 639, 660 on the small board 641 that are connected via the connector 637 for Vx.

Alternatively, instead of the monitor 668, the monitor 770 with electrodes shown in Fig. ? is used.

The switches 700 Kranz's terminal/Wilson's terminal switches the connection of the electrode 653 for Kranz's terminal/LL to the connection for LL and further connects the Wilson's terminal 428 to the inputs of both operational amplifiers of the monitor 349. The base 667 is moved from the position on the chest, shown in FIGS. 50B, 50C to the abdominal. area to the position of the Wilson's terminal 901 for sensing of LL by the “electrode 653 for Kranz's terminal/LL” and for applying of a reference RL by the “electrode 652 Vx/REF”, as it is shown schematically in FIG. 50D. Optionally, it is possible to use the switch 722 (I, II)/Vx, which switch allows sensing of Vx in the position B and by switching to the position A also to sense leads I and II and to calculate the leads III, aVR, aVL and VF from them.

In case the switch 722 is not used, the contact B—permanently—is connected to the contact C in the block diagram, and it is possible to read only the leads Vx against the Kranz's terminal or the Wilson's terminal.

Optionally, instead of the switch 722, two further operational amplifiers 426, designated OA4 and OA5, can be used, by which I and II leads can be sensed continuously, and 4 leads can be calculated from them. When sensing leads I and II, the switch 700 Kranz's terminal/Wilson's terminal is switched to position C for the Wilson's terminal and base 667 is moved to the abdominal region, so that the electrode 653 senses LL.

In the basic version, two operational amplifiers are used, namely OA1 and OA2, for sensing of the chest electrodes Vx and Vx+1 by means of a small board 641. The switch 722 is not used. By adding a third operational amplifier 426, OA3, it is possible to sense three chest electrodes Vx, Vx+1 and Vx+2 at the same time by means of the small board 725 of the three chest electrodes.

By adding further two operational amplifiers 426, OA4, OA5, it is possible to sense leads I and II at the same time as the three chest leads. By using only one operational amplifier 426 OA1, for example when using the monitor 770 of FIG. 54, only one chest lead Vx can be sensed simultaneously, preferably by the small board 654 of the electrode on cable, and by moving it all left, right and back chest leads can be sensed sequentially and when using the switch 722 also the lead I, and consecutively gradually also the lead II by adding the switch 654 LL/RA and from them some software can select pulse sequences with the same heart rate, from them determine the them corresponding sequences for other four leads III, aVR, aVL, aVF, and so to complete the ECG record of individually sensed leads to obtain a complete sequence corresponding to a twelve-lead ECG. To use the reference electrode 644, the cable 907 for a connector is inserted into the reference connector 664, and thereby, this disconnects the artificial reference and connects the electrode 644, as it is shown in FIG. 38.

FIG. 50B shows a procedure for sensing of chest leads using a base 667 of individual chest leads with the help of the Vx/REF electrode 652 against a Kranz's terminal. The base 667 is in the position 899 Kranz's terminal. The switch 700 Kranz's terminal/Wilson's terminal is in the position 1 for Kranz's terminal and the switch 638 is in the position 1 Vx.

The electrode 653 for Kranz's terminal/LL is placed in the area 44 of the Kranz's terminal and the electrode 652 Vx/REF is moved to the positions recommended for sensing of V1 to V6 and V1R to V6R.

FIG. 50C shows a procedure for sensing of chest leads using a Kranz's terminal base 667 of chest leads in pairs using a small board 641 and with the possibility to use a reference. The base 667 is in the position “position 900 of the Kranz's terminal with small board”, the switch 700 Kranz's terminal/Wilson's terminal is in the position 1 for the Kranz's terminal, switch 638 is in the position 2 REF. The base 667 is placed with the electrode 653 on the lower edge of the Kranz's terminal area 444 and the electrode 652 Vx/REF downwards to the abdominal area. This electrode is used for reference. Attached to the connector 637 of the base 667 is a small board 641 for a two-channel monitor with two electrodes for sensing of Vx and Vx+1. This small board allows sensing of any two adjacent leads Vx against the Kranz's terminal at once by means of a two-channel monitor 349, preferably formed by a monitor with an output for the reference 668.

FIG. 50D shows location of the base 687 for sensing by means of the Wilson's terminal in the position 901 of the Wilson's terminal and of the electrodes 194, 195 in the armpit, the small board 641 on the chest, and the position of switches 638 in the position 2, when the electrode 652 serves as reference RL, the switch 700 Kranz's terminal/Wilson's terminal is in the position 2. when the electrode 653 is used for LL, to switch the base from sensing with the Kranz's terminal Kranz's terminal to sensing with the Wilson's terminal. The base 687 is moved to the abdominal region into the position 901 of the Wilson's terminal to provide a RL reference using the Vx/REF electrode 652, which is switched to REF, and sensing of LL using the electrode 653 for Kranz's terminal/LL, which is switched to LL. The switch 700 for Kranz's terminal/Wilson's terminal is in the position 2 for Wilson's terminal, the switch 638 is in the position 2 REF. Preferably, the base is held by hand or it is fastened to the tape 732 for the base. The RA and LA signals are fed to the base 687 via the Wilson's terminal connector 701, the cables 640 from the electrodes 143, preferably formed by the electrodes 194 RA and 195 LA in the armpit, and preferably, they are formed by the voluminous electrodes 702. The chest and the back leads are sensed sequentially by applying the small board 641 so as it is shown in FIG. 51. The switch 638 Vx/REF is in the position 2 Ref, the switch 700 is in the position 2 for the Wilson's terminal.

FIG. 51 shows an example of changing of the ECG sensing with the Kranz's terminal to the Wilson's terminal by relocation of the monitor 349 from the base 667 for the Kranz's terminal shown in FIG. 35, located on the chest, to the base 705 for LL, RL, located at the waist.

The electrodes 143, preferably formed by the electrodes 194 for RA and 195 for LA, are in this example located in the armpit, preferably they are formed by voluminous electrodes 702, preferably formed by cylindrical electrodes 703, or spherical electrodes 704, or cuboidal electrodes 737, and preferably they are made of some soft, flexible material, e. g. they are made of foam rubber, so that its springing allows to move the arms away from the body without losing contact of the electrodes with the body or their falling out.

The voluminous electrodes 702 are preferably oriented so that their active conductive portion faces to the trunk, and senses so a signal only from the chest, where there are fewer interfering signals than in the limbs.

The voluminous electrode 702 consists of some flexible electrode pad 730 and the electrodes and the conductive portions 731 of the electrode cover preferably a portion of the pad 730. The electrodes 702 are connected by cables 640 to a finger-held “base 705 for LL, RL” or they are attached to the “tape 726 for RL, LL” in the waist area or this base 705 is inserted under the belt 733 of trousers and preferably it is secured by a clip 706 to the belt, or is inserted behind the panties 734 of the underwear.

The cable 640 supplies signals from electrodes 143 to the base 705, where the monitor 349 is located. Preferably, the electrodes 143 are formed by electrodes 639 for Vx and 660 for Vx+1 for the chest leads Vx and Vx+1, located on the “small board 641 of electrodes for a two-channel monitor.” The cable 640 is long enough or stretchable, so that the small board 641 reaches not only to the chest, but also to the back leads V7 to V9, where they are held by hand after placement, and preferably, they are fixed in the desired place so that, for example, the monitored person rests against the chair backrest. Alternatively, the monitor is placed on the base 641.

In these arrangements, the leads V1 to V6, V1R to V6R, V7 to V9 are sensed by placing the small board 641 at respective sites for sensing of pairs of these leads.

FIG. 51A shows shape adaptation of the “base 705 for LL, RL” for positioning of the monitor and inserting it behind the panties 734, which positioning provides pressure on the electrodes 143 for RL and LL. A fitted monitor 349 or a clip 706 prevent falling of the base 705 under the belt. Preferably, the base is fixed in the required position by the clip 706 on the belt.

FIG. 51B shows positions of hands for sensing of chest leads against the Wilson's terminal using at least one model of electrodes for LA, RA from: a shoulder wristband 736, a wrist wristband 735, a voluminous electrode 702, a glued electrode 1074, a contact electrode 1073 for finger, shown in Detail 1.2. The monitored person places itself the respective electrodes, such as voluminous electrodes 702 in the armpit or it touches with fingers the contact electrodes 1073, 194 RA and 195 LA, the voluminous electrodes 702, the glued electrodes 1074, the arm wristband 736, the wrist wristband 735 that are preferably connected with the base 705 by means of a connector 1070 of the jack model, which connector during inserting of the pin 1075 disconnects the contact electrodes 1073, preferably formed by the electrodes 194 RA and 195 LA on the base 705, by the switch 1076 of the connector, and connects the respective electrode connected by the cable 1014.

If the monitored person does not use the option to insert the base 705 behind the belt, so the monitored person holds it, preferably with his/her right hand on the abdomen. Preferably, it moves the small board 641 with two electrodes to the respective chest lead sensing points Vx with his/her left hand.

An advantage of the voluminous electrodes 702 is that they are flexible and thus they change their shape. They adapt to the degree of grip between the arm and the chest, and therefore, they are not annoying to the monitored person, they allow a wide range of arm movement and at the same time they ensure the necessary permanent contact with the skin of the chest.

Alternatively, instead of voluminous electrodes 702, wrist wristbands 735 or arm wristbands 736 may be used for LA, RA, which are preferably connected by a cable to a jack connector 1070 which disconnects the LA electrodes 195 on the board 641 held by the fingers of the right hand when inserted into the opposite connector, or the electrodes 194 RA are held on the base 705 by the fingers of the left hand. When connector 1070 is not connected, the electrodes 194 RA and 195 LA are used for RA, LA.

The wristbands 735, 736 are elastic, preferably of elastic material, preferably of rubber, or of metal plates 1049, which are attracted to each other by springs, so that the wristband 735 springs and rests on the skin, or they are provided, for example, with a Velcro for belting, and they are made of conductive material to realize contact with the body. In this example, the monitor 349 is a two-channel one or a single-channel one, preferably consisting of the monitor 770 with electrodes, which monitor is shown in FIG. 54. Therefore, only one operational amplifier 626 is used, wherein, it is labeled OA1 and in sensing of individual chest leads Vx, the small board 654 is used.

FIG. 51C shows a block diagram of the change from Kranz's terminal to Wilson's terminal sensing by moving the monitor 349 from the base 420 to the “base 705 for LL, RL”. The signals from the electrodes 639 for Vx and 660 for Vx+1 are fed to a monitor 349 connected to the base 705 for LL, RL by connecting elements 486 preferably formed by snap fasteners 487. The chest leads Vx are sensed against the Wilson's terminal. Therefore, a signal from the Wilson's terminal circuits 723 generated by the signals from the electrodes 194 RA, 195 LA, 197 LL is also applied to the monitor 349.

At the same time, a feedback reference signal from the input circuits of the monitor is used, which is fed to the electrode 196 RL.

The switch 722 of I, II/Vx leads switches the signal to the monitor 349 from the position B, i.e. from the electrode 639 for Vx and the electrode 660 for Vx+1 to the position C, i.e. to the electrode 194 for RA and the electrode 197 for LL and from the Wilson's terminal 428 to the electrode 195 for LA for sensing of ECG leads I and II, from which leads the leads III, aVF, aVR, and aVL can be calculated, thus obtaining a total of 6 ECG leads is obtained in this position of the switch 722.

In the position of the switch 722 for Vx, i.e. in the position B, with the gradual repositioning of the base 707 for the Wilson's terminal with two electrodes 639, 660, by means of which it is possible to sense by pairs of leads V1 to V6, V3R to V6R, V7 to V9, i.e. 13 chest leads. The leads V1R and V2R can be derived from the leads V1 and V2, what increases the number of chest leads to 15. The other two leads are sensed in the position of the switch 722 for leads I and II, from which leads III, aVF, aVL and aVR are calculated, i. e. four further leads, together 6 leads. Thus, the total number is 21 leads, which are sensed sequentially by a two channel monitor 349 shown in this example in approximately ten places. If a multi-channel monitor 349 and a “base 707 for the Wilson's terminal” would be the choice, for more electrodes 143, the number of relocations would be correspondingly lower.

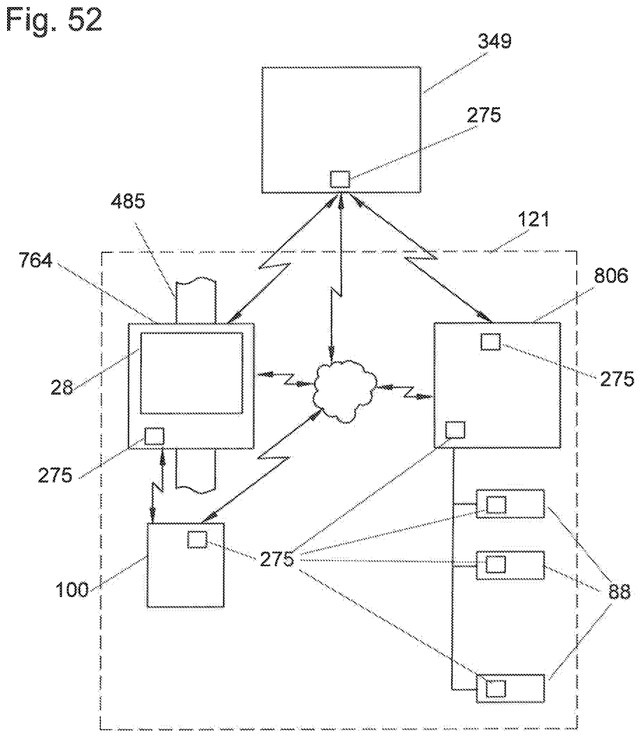

FIG. 52 shows communication of the units ensuring the operation and full use of the pocket monitor 349. The monitor 349 communicates, preferably wirelessly, by means of the communication unit 275 with the cooperating units 121, which are preferably formed by the evaluation and display unit 764.

Further, preferably, it communicates with the server 806, particularly via a mobile operator's networks 898 or a Wi-Fi network 131, or via a direct wireless link. Preferably, the evaluation and display unit 764 is formed by a mobile phone 100, or is connected to the mobile phone 100 on the wristband 485, preferably wirelessly. The evaluation and display unit 764 displays the results on the display 28. Preferably, it also controls the monitor 349. Further, the unit 764 is preferably adapted for direct wireless connection to the server 806. The processed sensed cardiac signals are also available on the server 806 for remote subscribers 88 and the operators of the server 806, who have access to the results and at the same time they can adjust the monitor, either directly or via the evaluation and display unit 764.

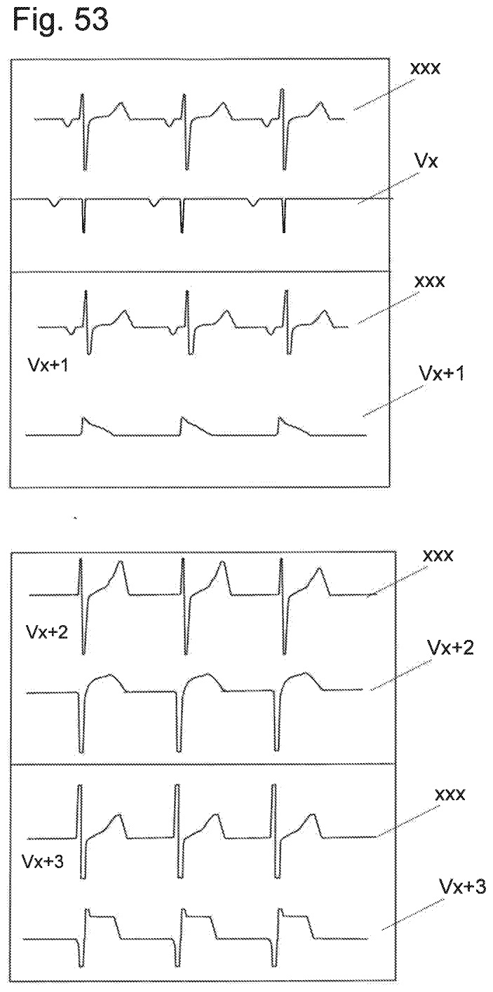

FIG. 53 shows a display of the evaluation and display unit 764, formed preferably by a mobile phone, where, for example, when using a two channel or a multi-channel monitor 349, each time cardiac signal data is transmitted from a location for certain chest leads Vx, preferably initialized by a display button 750, the monitor 349 or the sensing unit 571 or the sensing and evaluation unit 764, preferably after each placement of the base in a new location on the chest or back, preferably displays two or more ECG curves for the leads actually sensed. Preferably above them or inserted into them, a master ECG curve is displayed, which curve shows the normal ECG curve for this or that relevant lead. Preferably, the normal ECG curve is recorded at a time when the subject monitored exhibits normal course or a course without any myocardial infarction (MI).

Accordingly, the monitored person evaluates, whether the sensed course differs, especially with respect to the elevation of the ST segment. If the actual course agrees with the master one, there is no suspicion of the possibility of a heart attack.

In case of difference, the monitored person evaluates the ECG according to the instructions, especially with regard to possible heart attack and/or sends the recording from the monitor 349 via the communication unit 275 or from the evaluation and display unit 764 directly or via server 806, and from there to the remote participants 88 who evaluate the recording or live transmission and notify the monitored person. Preferably, the ECG image can be transmitted from unit 764 preferably represented by the mobile phone, as an image of the display as a MMS.

Preferably, each captured ECG record for individual leads is stored in memory and the unit 764 is adapted to sequentially display the selected leads, preferably by scrolling with a pushbutton or, in the case of a touch keyboard, with a finger.

The data transfer from the monitor, preferably to the unit 764, is preferably initiated by the transfer pushbutton 753, preferably after each application or relocation of the sensing unit 571 to the desired cardiac sensing location.

FIG. 54 shows a sensing unit 571 on the chest belt 749, preferably formed by a strip-shaped multi-electrode base 630, preferably for sensing from electrodes 194 RA and 195 LA and with a strengthened Kranz's terminal sensing up to 9 chest leads V4 to V9 and V4R to V6R, wherein all electrodes 143 are placed on the chest belt 749. To place all electrodes for the 21 lead ECG on the chest belt 749, a strengthened Kranz's terminal 692 or a pseudo-Kranz's terminal 693 is used as a replacement instead of electrode 197 LL, the electrode 196 RA is placed on the chest belt 749, together with electrodes for V1 to V3, V1R to V3R. This allows electrodes V3 to V9 and V3R to V6R to sense leads V1 to V9, V1R to V6R. The lead I of the ECG is sensed from the electrode RA against LA, and the lead II of the ECG is sensed from the replacement for LL, i. e. from the pseudo-Kranz's terminal 693, wherein, 4 leads are calculated from the leads I and II, a total of 21 leads, i.e. V1 to V9, V1R to V6R, I and II, and 4 calculated leads.

It is possible to supplement the ECG electrodes 143 located on the belt 749 with electrodes located outside the belt 749, namely the electrode 197 LL, preferably by means of a bridge for sensing by means of a pseudo-Wilson's terminal 694, see FIGS. 43,47 and electrodes 194 RA, 195 LA that make possible to sense by means of the Wilson's terminal 428, as it is shown, for example, in FIGS. 45, 49, 55, preferably by means of a bridge 683.

The interconnecting field 711 connects the respective electrodes 143 to at least one monitor 349 for sensing of the selected leads. The multi-electrode base 630 is attached to and held on the chest and back, preferably by an extension 778 for arm, which presses the belt 749 against the chest in the direction of the arrow 762 in the direction of a hand pressure and the base extending portion 682, preferably formed by a brace 338 held by leaning of the monitored subject against chair backrest, as it is described in the example shown in FIG. 44. Alternatively, the multi-electrode base 630 is permanently attached to the trunk by the resilient portion of the belt 82 which is clipped to the belt 749, and the extension 778 is disconnected.

To obtain strengthened terminals of the Kranz's terminal 692, electrodes 585 LA′ and 586 RA′ are used, which are located on the sides of the multi-electrode base 630 at the places of sides of the monitored person, which are interconnected by means of three resistors 425 with the Kranz's terminal, as it is shown in FIG. 42.

Alternatively, a pseudo-Kranz's terminal is preferably used, which is obtained using 2 resistors 425 from electrodes 585 LA′ and 586 RA′ located on the chest belt 749 according as shown in FIG. 42A, or the electrode 197 for LL for the Wilson's terminal is connected, so that the electrode 481 for the Kranz's terminal does not need to be placed on the multielectrode base 630, instead of which it is possible to place the electrodes 211 V1 and 212 V2 alias V1R, V2R, shown in dashed lines, which are added to the eleven ones for the chest leads above, whereby a complete set of 15 chest electrodes is obtained.

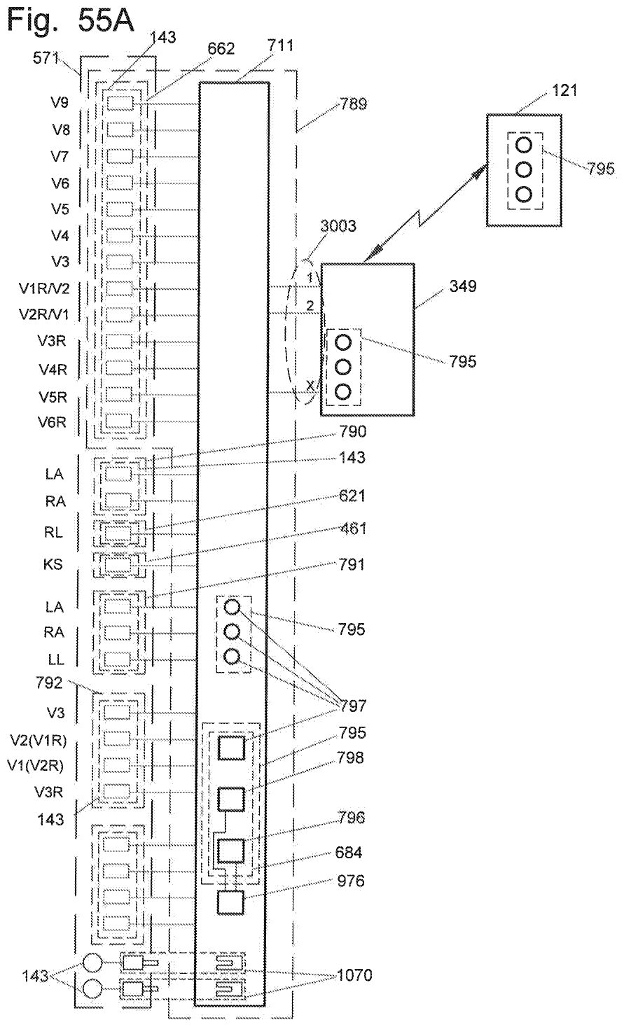

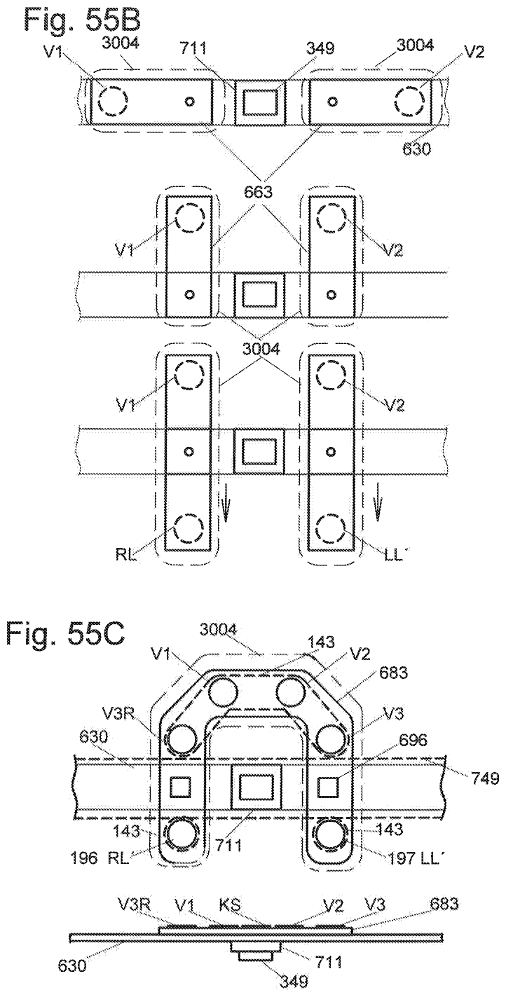

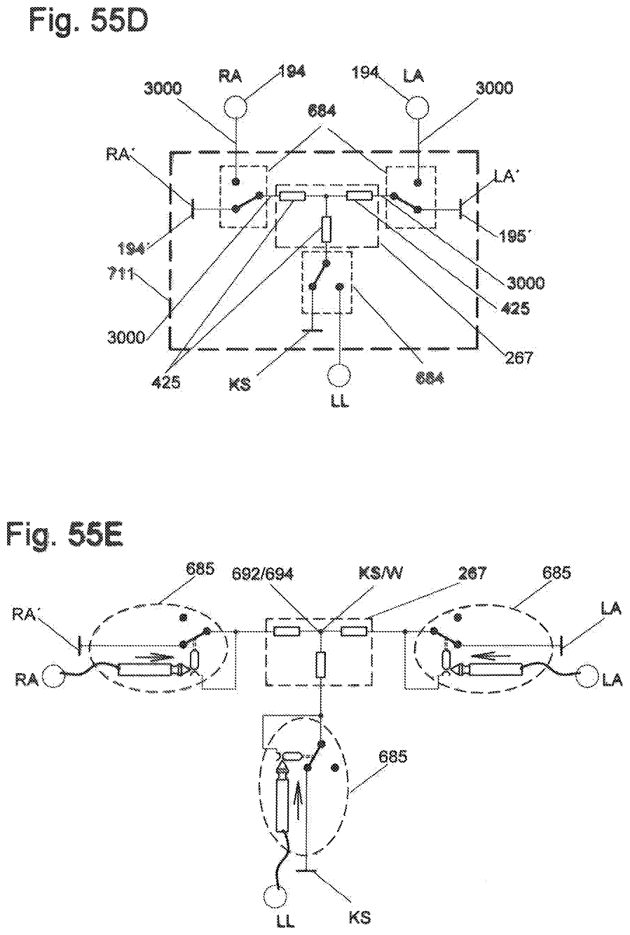

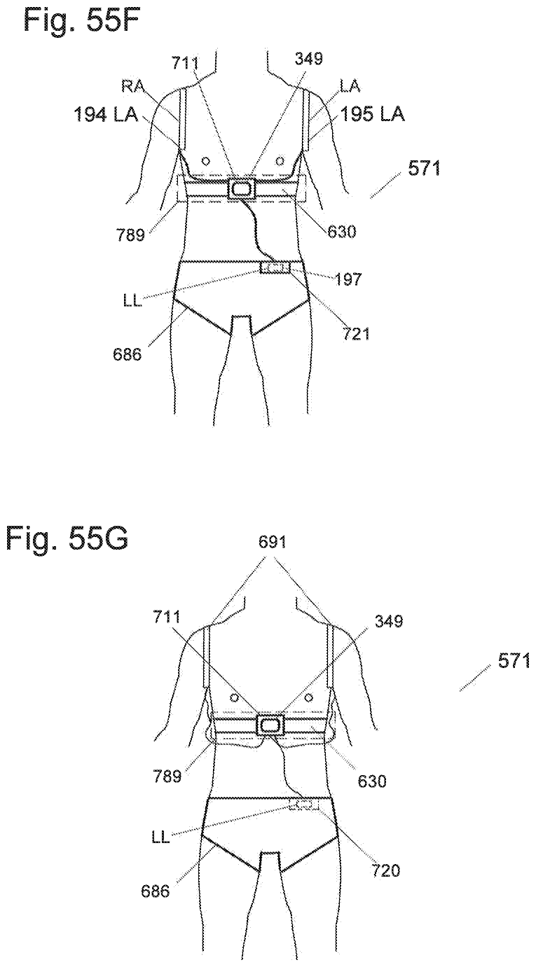

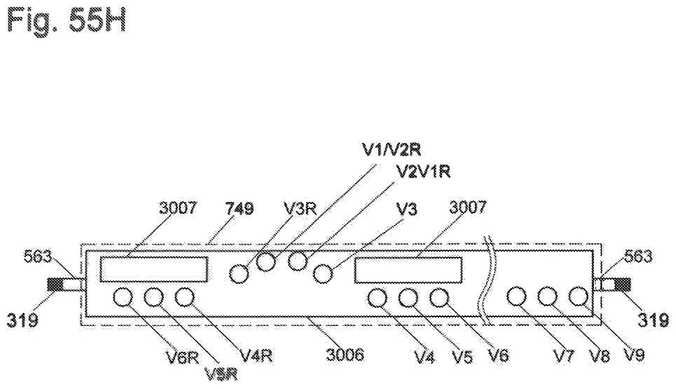

FIG. 55A shows electrodes 143 which are applied to the interconnecting field 711 which field connects them to at least one monitor 349 for sensing of selected leads by means of a switch 684 of the interconnecting field, preferably formed by manual switches 797 and/or electrical switches 796, preferably formed by pushbuttons or electronic switches 798 controlled by the control unit 976 of the interconnecting field, preferably controlled from controlling elements 795 of the interconnecting field, which are located in the interconnecting field 711, or on the monitor 349, or in the cooperating units 121. The electrodes 143 are located in or off the sensing unit 571, preferably formed by base 789 in the form of a belt, preferably formed by a multi-electrode base 630 or by one of the bases 420, 620 for RL, 712 complete, 765 in the form of a belt, simplified 720.

The electrodes 143, which are located on the base 789, are preferably formed by “electrodes 662 for the chest lead”, “electrodes 790 for the pseudo-Wilson's terminal”, “electrodes 621 for RL”, “electrodes 461 for sensing of the Kranz's terminal potential”, and electrodes 143 located outside the base. 789, which are preferably formed by the “Wilson's terminal electrodes 791”, “chest lead electrodes 792,” alternative electrodes 793, see FIGS. 55B-F.

FIG. 55B shows the extension of the number of electrodes 143 at the base 789 by electrodes for leads V1, V2 to the leads sensed according to FIG. 55, which are located on the rotating strips 663. Both rotating strips 663 are composed of two segments, the upper one and the lower one. In the working position described below, the joined upper and lower segments are tilted upwards, and in this position the bottom segment is moved out downwards. In this configuration, they snap and form a strip perpendicular to the longitudinal axis of the multi-electrode base, which is the base pressed against the chest. In the upper part of this Figure, they are drawn in the unused position, in which position they are turned into the plane of the multi-electrode base 630, in the central part of this Figure they are turned upwards by 90° to be brought into the working position, whereby the electrodes for V1, V2 get into the sensing position, and at the bottom of this figure also the lower parts of the tilting straps 663 are moved out, what pushes the electrodes for V1 and V2 against the body by ensuring that the straps 663 are attached to the chest so that the electrodes V1, V2 cannot be tilted away.

The electrodes for V1, V2 are connected to the monitor 349 by an interconnecting field 711, as well as, preferably, the electrode for LL is connected instead of the electrode for the Kranz's terminal, thus obtaining signals, which lie approximately somewhere between the signals that we would obtain by sensing against the Wilson's terminal and the Kranz's terminal. By connecting the LL electrode 197 and by disconnecting the LL′ electrode, it can be sensed against the Wilson's terminal.

FIG. 55C shows the wiring of electrodes 143 that are located outside the multi-electrode base 630, preferably formed by a chest belt 749, for V1 to V3 and V3R, and preferably electrodes 143 for LL′, RL on a bridge 683 optionally attached to the multi-electrode base 630 by a connecting element 696 to achieve placing of electrodes 143 on the chest for the prescribed spots for ECG sensing. The electrodes 143 are connected by interconnecting field 711 as needed.

FIG. 55D shows connection of the external electrodes 143 for LA, RA of FIG. 55 instead of electrodes 143 for LA′, RA′ located on the multi-electrode base 630, and connection of the electrode 143 for LL instead of electrode of the Kranz's terminal to achieve full valued sensing of chest electrodes against the Wilson's terminal and sensing of the leads I and H of the ECG with the possibility of calculating another 4 leads, whereby up to 21 lead ECG is obtained, which is suitable for diagnosis of infarction also from the left hand side and back leads. The electrodes 143 are connected by switches 684 of the interconnecting field 711. Optionally, the switch 684′ disconnects the electrode for both the Kranz's terminal and the LL for sensing using the pseudo-Kranz's terminal 693 shown in FIG. 42A.

FIG. 55E shows an alternative connection of the electrodes 143 for LA, RA, LL by means of the switching connector 685 of FIG. 60, which, when inserted, connects the electrodes, for example the LA one and disconnects the LA′ one, and similarly for the RA, LL.

FIG. 55F shows connection of the electrode 197 for LL, which is located on the abdominal small board 721 to change sensing by means of the strengthened Kranz's terminal 692 for sensing by means of the pseudo-Wilson's terminal 694.

FIG. 55G shows a belt-shaped base 789, preferably formed by a multi-electrode base 630 when connecting the external electrodes 143 for RA, LA, LL, where for RA, LA shoulder straps 691 with an electrode have been selected as an example, and for LL an abutment small board 720 inserted under the underwear 686.

Thus, in this exemplary embodiment, the electrodes 143 are implemented as an advantageous alternative to the glued electrodes, so that the use of them can be avoided.

For sensing with a full-valued Wilson's 428 terminal, it is sufficient to connect to the multi-electrode base 630 the electrode 197 for LL, for example, placed on the abdominal small board 721 and to carry out connection of the electrodes 194 RA and 195 LA preferably on shoulder straps 691, on glued electrodes, or on electrodes on terminals.

Advantageous is that, apart from the electrode 197 LL and the electrodes 194 RA, 195 LA, all electrodes 143 for obtaining of sensing 21 of leads on the multi-electrode base 630 are in the form of a belt.

FIG. 55H shows a wide chest belt 749 which allows to place the electrodes 143 for sensing of the chest leads to the optimal positions situated on the curve as shown in the Figure. A chest belt of such width would cover also the nipples and it could cause irritation of skin. Therefore, openings are shaped in the chest band to maximize the surface of the skin not covered by the chest band, especially in the nipple area. The figure shows the holes 3007 designed exactly for the nipples. At the ends, the wide chest belt 749 is provided with fixtures 563, preferably formed as Velcro 319, for quick connection and fastening of the chest belt 749 around chest of the subject being monitored.

### INDUSTRIAL APPLICABILITY

This device is applicable in the boundary region between the consumption and the medical techniques.

1. A pocket ECG for the diagnosis of infarction, characterized in that,
it comprises a sensing unit (571) formed by a chest belt (749) for sensing of at least twelve ECG leads, wherein, all ECG electrodes (143) that are necessary for sensing of the twelve ECG leads are located on the chest belt, or a part of them is located on at least one of the parts 3004 for fastening of the electrodes that are connected removable to the chest belt (749) being integral with the chest belt, or the electrode (197) for LL is placed on a small board (654) of an electrode connected to the chest belt by a cable,
a pocket monitor (349) of cardiac signals placed on the body or on/in clothing of the monitored person, which is adapted for simultaneous processing of cardiac signals for at least twelve ECG leads, and this of all ECG leads that the sensing unit (571) is adapted to sense,
or the monitor (349) is adapted for simultaneous processing of cardiac signals for fewer leads than the sensing unit (571) is adapted to sense, and the monitor is connected to the sensing unit (571) via an interconnecting field (711) which field connects the monitor (349) at first to that number of electrodes (143), from which the monitor is adapted to process cardiac signals, and subsequently it is sequentially connected to further electrodes (143) of the sensing unit (711) for sequential processing of further cardiac signals until all cardiac signals that sensing unit (571) able to sense are processed, whereas
the processed cardiac signals are fed from the monitor to at least one of a display (28), a memory (961), a communication unit (275), and
the display (28) shows an ECG processed by the monitor (349) from the cardiac signals and the memory (961) is used to store the cardiac signals processed by the monitor (349) and the communication unit (275) is determined for transmitting of the cardiac signals processed by the monitor (349) to the cooperating units (121).

2. A pocket ECG for the diagnosis of infarction according to claim 1, characterized in that,
the cardiac signals for at least twelve ECG leads are sensed from the electrodes (143) of ECG for the lead I formed by electrode (194) for RA and by electrode (195) for LA, for the lead II formed by the electrode (194) for RA and by the electrode (197) for LL, or by connecting the pseudo-Kranz's terminal (693), which replaces the electrode (197) for LL, and for the chest leads V1 to V6 by electrodes for the leads V1 to V6 against the pseudo-Kranz's terminal (693), wherein the ECG leads III to IV are calculated from the ECG leads I and II.

3. A pocket ECG for the diagnosis of infarction according to claim 2, characterized in that,
by connecting of the electrode (461) of the Kranz's terminal via a resistor (425) to the pseudo-Kranz's terminal (693), it becomes a strengthened Kranz's terminal (692) and
by connecting the electrode (197) for LL via a resistor (425) to the pseudo-Kranz's terminal (693), it becomes a Wilson's terminal (428), wherein,
the reinforced Kranz's terminal (692) or the Wilson's terminal (428) replaces the pseudo-Kranz's terminal (693) for more accurate sensing, wherein the cardiac signals sensed from the reinforced Kranz's terminal (693) or the Wilson's terminal (428), the electrodes (143) for the chest leads V1 to V6 serve to be processed by the monitor (349) to chest leads V1 to V6 of ECG, and the reinforced Kranz's terminal (639) replaces the LL for sensing of cardiac signals against the electrode (194) of RA for processing to II lead of ECG.

4. A pocket ECG for the diagnosis of infarction according to claim 1, characterized in that,
the parts (3004) for mounting of electrodes consist of at least one of: a board (710), a segment (321) of electrodes, a rotatable tape (663), a bridge (683).

5. A pocket ECG for diagnosis of infarction according to claim 1, characterized in that,
for placing of all electrodes on the (749), this chest belt is formed by a wide chest belt (3006), where the electrodes (143) for V4 to V9 and the electrodes V4R to V6R are in one plane in the bottom part of the wide chest belt (3006) and the electrodes (143) for V1 to V3, and for V1R to V3R are located in an arc in the upper part of the belt (3006) in the corresponding places for their sensing, wherein, for nipples, preferably, a hole (3007) is formed, behind which the lower and upper parts of the belt are again connected, the waist is wrapped around the chest and its ends are connected together by a fixture (3009) with an adjustable belt length.

6. A pocket ECG for diagnosis of infarction according to claim 1, characterized in that,
by adding electrodes for leads V1R to V6R and leads V7 to V9 to electrodes for leads LA, LA, LL, RL and chest leads V1 to V6 on the chest belt (749) or parts (3004) for fixing of electrodes, capacity of the sensing unit (571) is expanded from twelve leads to a maximum of 21 leads of ECG.

7. A pocket ECG for diagnosis of infarction according to claim 1, characterized in that,
the chest belt (749) comprises a multi-electrode base (630) with electrodes for RA and LA on the sides of the chest belt (749) or on the removable side straps, the right strap (632) and the left stripe (633), wherein, electrodes (143) for the chest leads V1 to V3, V1R to V3R, are located on the chest belt (749) and the electrode (197) for LL is replaced by a reinforced Kranz's terminal, or a pseudo-Kranz's terminal in case that the bridge (683) is not used, and if it is used, the electrodes for the chest leads and the electrode (197) for LL are located on the bridge (683), or the electrode (197) for LL is alternatively located on the left side belt (633), the or on the small board (654), wherein, the bridge (683) is removable and the electrodes (143) for V4 to V6 on the left-hand side, for V4R to V6R on the right-hand side, and for V7 to V9 on the back and for RL are located on the chest belt (749), wherein the electrode (143) for RL is alternatively located on the right-side side strap (632) below, if it is used, what means that for placing of all electrodes on the chest belt (749), electrodes V1 to V9, V1R to V6R, RA, L are placed on it, and for replacement of the electrode (197) for LL the reinforced or pseudo-Kranz's terminal is used for sensing of leads I, II, V1 to V9, V1R to V6R, and four leads are calculated from the leads I and II, for a total of 21 leads, wherein for a more accurate sensing parts (3004) are used, for example a bridge (683) for placing of electrodes for V1 to V3, V1R to V3R, LL, where it is possible to process cardiac signals to an ECG curve using a Wilson's terminal (428).

8. A pocket ECG for the diagnosis of infarction according to claim 1, characterized in that,
the chest belt (749) is adapted for permanent attachment to the chest for permanent monitoring or for a short-term attachment, wherein, preferably it comprises a brace (338) for comfortable inserting of the chest belt (749) under the shirt on the back, where by pushing it towards the chair backrest, the belt is fixed on the side and by leaning of the arm against the extension (778) also on the other side for fixing of the chest belt (749) in a stable position relative to the chest for quality sensing from the electrodes (143) for ECG without interference caused by shifting of the electrodes (143) against the chest.

9. A pocket ECG for the diagnosis of infarction according to claim 1, characterized in that the monitor (349) is connectable to the sensing unit (571) by means of connecting elements (486), what allows moving of the monitor (349) between the sensing units (571).

10. A pocket ECG for diagnosis of infarction according to claim 1, characterized in that that the interconnecting field (711) comprises mechanical and/or electronic interconnecting elements.

11. A pocket ECG for the diagnosis of infarction according to claim 10, characterized in that the electronic interconnecting elements are controlled by means of the control unit (767) of the interconnecting field by controlling elements, which are located on the sensing unit (571) and/or on the monitor (349), or they are controlled from the cooperating units (121).

12. A pocket ECG for the diagnosis of infarction according to claim 1, characterized in that
that the monitor (349) is interconnected by means of an interconnecting field (711) to the sensing unit (571) with electrodes (143) for ECG, wherein, the single-channel monitor (349) is interconnected by means of an interconnecting field (711) with the electrodes (143), what allows sequential sensing of individual analogous cardiac signals for sensing of up to 15 chest leads, two limb leads I, II, and processing of analogous cardiac signals by the monitor (349) into digital form in the form of data for digital transmission and/or for displaying, wherein, the dual-channel monitor (349) allows to sense signals for leads I and II simultaneously, and to calculate four leads III, aVF, aVR, aVL from them,
and further it allows switching from sensing of the leads I and II to sensing from chest leads for sensing of up to 15 chest leads, always two leads at a time, and
a three-channel monitor (349) allows to sense leads I, II and to calculate four leads from them and one chest lead at the same time, wherein, after switching by the interconnecting field (711), from sensing of leads I/II for sensing of the chest leads, it is possible to sense up to 15 chest leads by successive switching of electrodes (143) from a set of chest electrodes (698), always up to three chest leads at a time, and an eight-channel monitor (349) allows to sense leads I, II and from them to calculate four leads, and further the left-hand side chest leads V1 to V6 simultaneously for a 12-lead ECG, wherein, for the right-hand side leads and the back leads it is possible to switch the respective electrodes via the interconnecting field (711) to the monitor (349) for sensing of up to 17 leads of ECG and to calculate further four leads and
the 17-channel monitor (349) allows to sense all 17 leads and to calculate 4 leads simultaneously for a complete 12-lead ECG plus 6 right-sided chest leads and 3 back chest leads, i. e. for 21 lead ECG.

13. A pocket ECG for the diagnosis of infarction according to claim 1, characterized in that the monitored person detects a suspicion of an actual myocardial infarction (AIM) according to an AIM change (742) in the ECG on the display of a monitor (349) or on the display of the cooperating units (121), and this from the elevation of the ST segment, enlarged T wave, the modified shape of the QRS wave with a loss of the R wave, or an inversion of the T wave, wherein, the ST elevation, as the most important indicator of an AIM, is at best seen on the chest leads of the ECG.

14. A pocket ECG for the diagnosis of infarction according to claim 13, characterized in that the monitored person detects the ST elevation (603) and other AIM changes (742) on an ECG by comparing the actual ECG (601) with a master ECG (602) provided by the monitored person at a time when there was not any AIM, which is displayed on the cooperating unit (121) and/or on the sensing and evaluation unit (764), wherein, the comparison is preferably performed each time the monitored leads are displayed sequentially, if the monitor (349) is adapted to process only a portion of the sensed leads by the sensing unit (571), or if the monitor (349)) is adapted to process all sensed leads, then according to the overall display of all leads on the display.

15. A pocket ECG for the diagnosis of infarction according to claim 14, characterized in that the master ECG (602) is displayed simultaneously with the actual ECG (601) for finding of the difference in ST elevation (603) and/or in other changes (742) of the AIM ECG, where when the actual ECG (601) matches the sample ECG (602) it means that when there is no apparent difference in the ST elevation (603) and/or there are not any different changes (742), there is not any suspicion of AIM, but when a change is apparent, the monitored person evaluates the change according to the instructions and decides whether to perform the evaluating conclusion himself/herself, or it transmits the ECG to professional medical staff.

16. A pocket ECG for the diagnosis of infarction according to claim 15, characterized in that the monitored person sends the ECG record to the medical professional staff via the cooperating units (121) by sending it to a PC (962) of the medical staff, or to a server (806), to which the remote participants 88 have access, such as the medical staff, or sends it to a PC (889) and from there to the medical staff, or sends the record from the unit 764, preferably formed by a mobile phone, as an image via MMS.

17. A pocket ECG for the diagnosis of infarction according to claim 16, characterized in that the transmission from the monitor (349) to the unit (764) is preferably initiated by a button (753) for transmitting, preferably, each time the monitor is switched to further electrodes (143) of the ECG sensing unit, wherein, each record is stored in memory and ??? units (764) is adapted for sequential displaying of selected leads, preferably by scrolling using a pushbutton or a touch display.

18. A pocket ECG for the diagnosis of infarction according to claim 16 or 17, characterized in that the monitor 349, the cooperating units (121) and/or the unit (764) are adapted for evaluating of AIM changes in an ECG and/or for triggering or comparing of the actual ECG (601) with a master one and to trigger an alarm in the event of an indication of AIM.
